(19) United States
(12) Patent Application Publication
(10) Pub. No.: US 2003/0054547 A1
(43) Pub. Date: Mar. 20, 2003

(54) Polyketide synthase gene from sorangium cellulosum

(76) Inventor: Bryan Julien, Oakland, CA (US)

Correspondence Address: Carolyn A. Favorito, Morrison & Foerster LLP, Suite 500, 3811 Valley Centre Drive, San Diego, CA, 92130, US

(21) Appl. No.: 09/942,025

(22) Filed: Aug. 28, 2001

Related U.S. Application Data
(63) Continuation-in-part of application No. 09/144,085, filed on Aug. 31, 1998.
Application No. 09/144,085 is a continuation-in-part of application No. 09/010,809, filed on Jan. 22, 1998.
(60) Provisional application No. 60/271,245, filed on Feb. 15, 2001.

(51) Int. Cl. C12N 15/00; C12N 15/74

(52) U.S. Cl. 435/320.1

(57) ABSTRACT

The tmbA gene cluster of Sorangium cellulosum encodes a PKS and modification enzymes for the biosynthesis of tombamycin.

### CROSS-REFERENCE TO RELATED APPLICATIONS

The present application is a continuation-in-part of allowed U.S. patent application Ser. No. 09/144,085, filed Aug. 31, 1998, which is a continuation-in-part of U.S. Pat. No. 6,090,601, each of which is incorporated herein by reference.

### FIELD OF THE INVENTION

The present invention relates to the fields of molecular biology, chemistry, and medicine.

### BACKGROUND OF THE INVENTION

Sorangium species produce a variety of useful polyketides, including epothilone, myxothiazole, and soraphen. U.S. Pat. Nos. 5,962,290; 6,066,721; and PCT patent publication Nos. 98/49315; 99/0398600/24907; 00/31247; and 00/44717 describe methods for making novel polyketides by combining portions of two or more polyketide synthase (PKS) genes to create novel genes that encode a hybrid PKS and by providing synthetic biosynthesis intermediates to modified PKS enzymes. There remains a need for new polyketide synthase genes for use in the preparation of hybrid PKS enzymes and the polyketides produced by such hybrid enzymes. The present invention meets that need by providing recombinant DNA compounds that comprise all or a portion of a PKS gene from Sorangium cellulosum.

### SUMMARY OF THE INVENTION

The present invention provides recombinant DNA vectors and host cells that comprise the tmbA genes of Sorangium cellulosum or fragments of those genes.

These and other aspects of the invention are described in more detail in the following description and claims set forth below.

### BRIEF DESCRIPTION OF THE FIGURES

FIG. 1 provides a physical map of the tmbA gene cluster and an alignment of the cosmids of the invention (34-7, 28-26, and 14H12) that comprise the tmbA gene cluster genes and gene fragments. The PKS genes are designated tmbA, tmbB, and tmbC. Open reading frames (ORFs) are designated 1 through 8, inclusive. ORFs 1 and 3 are overlapping. ORF 1 is a thioesterase (TE); ORF 3 is a methyltransferase. ORF 4 is a hydrolase. ORF 6 is an epoxide hydrolase.

FIG. 2 provides a structure of the polyketide tombamycin produced by the TmbA PKS in monomeric and dimeric form. R is a is a substituted or unsubstitued C₃-C₈ alkyl or cyclic alkyl.

### DETAILED DESCRIPTION OF THE INVENTION

The present invention provides recombinant DNA vectors that comprise all or a portion of any of the genes in the tmbA gene cluster. The tmbA gene cluster is comprised of PKS genes tmbA, tmbB, and tmbC, and ORFs 1-8, inclusive. Each PKS gene in the cluster is composed of one or more PKS modules, each comprising an acyltransferase (AT), ketosynthase (KS), and acyl carrier protein (ACP) domains and optionally one or more ketoreductase (KR), dehydratase (DH), and enoylreductase (ER) domains as well as linkers that connect one domain to another and one module to another. The boundaries of each of these domains can be identified by sequence comparison with known PKS genes and enzymes. In one important embodiment, the invention provides recombinant DNA vectors that encode all or a portion of one or more of these domains that are useful in the construction of hybrid PKS genes and enzymes.

The sequence of the tmbA gene cluster is shown below.

CTCCAGATCGACCTGCATGATCTTGCCGACGAGCTGCAGCAGCTCAGGGTCCTCCTGGAT
CACACGATCGAACGCCCCGTACCTCTGCTGTACACGCCGCAGGAACTCGCTCGCGGGGAG
GTGAGAGATCGGCGGCAGGCGGGCGGGTACGTGAGGGGCTCTGCTCGCGGCGACGACCAT
GCATGTCGGCTCGATCCCACGGCGCCTGCGGAGAGAACGGGCCCATTCGAAGGCCATCAG
CGCTCCCAGGCTGTATCCAAAACACGCGAACGGCAGGTCGAGCATGTCATCCAGCCCCCG
CTCGATGCCGTCGATGAACTCCGACATCGAGCGAGGTTGCTTCTCCTTGGACCGAGCGAA
TCGCCCCGGAGGCTCTATCGGGCAGACGTCGATATGCGCCGGTAGGCTCTTCGCCCAGTC
TCGATATATGGCCCCGGCCGGCGCCGGCGTAGGGAAGCAGAAAAGGCGAAGCTTCGCGTC
CGTCCGCGCGGTCCAGTGCGACAGCCAGGGATTCTTCTCCATGTAGACCTCCGGTGCAAG
ATGATCGTGTCTCTCGCGCGAGAGCGATCATGGTCAAAGCCATTTGCATTGGTTGCGATT
CGCGTCAACCGGCTCCGTGAGAGGCGATACGACCTCACGGCGAAGGCGGCATCCCGATGC
CGGCGCCACGACCAATGGGGCGCCGATTCTATCCATCCAGCCTCGCACACGATTTCGATG
CTCGTCGGTATCTCAGGTCCATTGACGAGGCAATGTATTCTATGTTAGGTGTTCCCATAG
AGACATCGCCATGAACACGCGAAAAATTCGTATCGTACTGTATGCCGCGCTCACGCTGAT
CGGCGCTGCCGGCGTTTTGTGGTTCAATATACGGTTCATGAACGCCGGGGGGTTCGATTC
ACCGACGGGGTTCGTCGACGCGGCCACGGTCAACGCGGCGGGCGCTTCCGTGTTCGTCGA
CATCGCGGTGGCCGCCACGGCTGGGATGCTGTTCATGGTGGTCGAGGGCCGGCGGGTGGG
CGTGCGCCATGTCTGGGCGTATGTGTTGCTGAGCTTTTTGCTCGCCTTCGCGGCGACGTT
CCCGGCGTTCTTGCTGGCGCGCGAGCTGCGCCTCGCTGCGCTCGAAGGGGCGGGGGGACG
CTGACGGCCCGGGCGGGGCGGCCCCCGGCGCGCTACGTGGAGCGCGCCGGAGAGACCGGC
CACTTGCGCCGGGAGGCCGCGCGCGGTGCTCACCTGGCGGAGTGGGGCATATTCCCGGGC
TTCTCTCCGGATGCGATGACGTAATCGAATGAGGCGAACAGCCAGGAGAGCAGCGGGTTC
TCCTTCATCAGCAGCAGCCGGAGCAACGGGCTCTTGTGCTTGAAGTCGGGGCTCGCCGAG
CGGCGATCCAGGTGGTCGAGGTACTGATCGTAGACGTGTTTGCGAATCGACCGGACCTCG
ACGTTCACGAAGCCGGCGGCGCGAAGCCTCTCCGCATAGACGTCGCGAGGGTAGTGGTTC
TCCGCGGGGGCTTGCGACCGAAGGGTGAACAGCGGGGAGCTCCCCCGCCCTGGCAGCGGG
ATGATGTCGGCGGTGGTGATTCGACCTCCCGGGCGGAGGACCCGAAAGGCTTCGCCGAAG
AATTTGTCGCGCGTATCGAAGTGAAAGGCCGACTCCACGGCGAGCACCTTGTCGAAGGTC
TCCGGCGCGAAGCGCATGTCCGTCGCCGAGCCCACGCGCAGGTCGATGCGGTCGTCGAGC
CCGAGCTCGGCGATCCGCCTGCGCGCCTCTTCTACCTGCTTCTCTGTAGACTTGATGCCG
ACGATGCGCTTCGGCGAGTAACGCGTTGTCCAGTGGATGTCCTGGTCGCCGAAGCCGAAG
CCCACGTCCAGCACCTCGTCGCTCCCCGAGAGCTTCGCGGCCTCTGCGACGAGGTCGACC
AACGCGGTCGACGCGGCGTCCATCGTCCTCGTCTCTGGGTCGGCCCAGTACCCGAGGTTC
AAGAACTTGGACTGCTCGGTGAATACGTTCTCGCCGAGGTTGTTGTAGTACCAGCGAGGG
TCCCTGCCCTTCGCGATGGTGTGCACCGACGTGAACACCCCGCTCAGCGCCGCGCGCCAG
TTCGCTCTATTCCGATGTATATCCATCCCGTCCTCATGGTGCCCTGGCGCATGGCCAGAG
ATTCGAGTCATCCCTCGATGCGGCGTTGCTCGCGACGATCGGGCGAGCCCGTACCGGTTC
GCCCTGACCGCCCCGGTCCCTGCCGGATCAGAAGTAGGGGTAGGCCGGCTCGATACCCAT
CAGCGCCTGGCCGATGATTGAGTATCTATTCTTCTGGCAGAGGAAGTGCGAGTTTGCGGT
GATGATGTCCCGCCGGAGGCGCTCGATCGGATGCTTGGCGAAGATCGCGGTGGCCCCTGC
CACGTCGCACATCGATTCGACCACGCGTCGAGTCGATTGTGCGGCGTTGATCATCATCAG
CACTCCTTTGGCGCGCTGCTTCAGCGACGGTCCGTTGCCGGATTGCAGCGCATCCCACAG
CTCGCCGACGACATCGCGCACGTAGCTCCTGGCCGAGCCCAACGTGGCCTCCGCCTCGGC
GATCGCTATCTGCACGCTCTGCTCGGTGCCCAGGTTGTGCCCCATCGGCATTACCTTCTT
GTTCTGCGCGAGCTCGAGCGCGATCTCGATCGCGTGGCGCGCGATGCCTAGCTGAACGCC
CACCAGGGTTTGCAGGAAGAGGGGGGCATAGGCGTACAGCGGCTCGCTGCGCTTGCGGTC
CGCCAGGTTGAAGGTGTGCTCCTCGGGCACGAAGACGTTGCTCGCGCTGCAGTCGTTGCT
ACCGCTTCCCGCCAGGCCCGTCGTGTGCCAGGTGTCCAGGATCTCGAAGCTTCCAGCGGG
GAGGACGGCCCAGAGGGTACGCGGGCTGCCGTCCGCGTTGAGAACCGGGCTGTCATTTTC
GGTGACCAGACAGCCCGTCGCAAGGAAGTCCGCGTGGTGGCAACCGCTGCAGAAGTCCCA
GGTCCCTGTCACCCGATAGCCTCCGGCCACCTTCTCAGCCTTCCCGGTCCACCTCAGGCT
TCCGGCGTGGCGTACGTCGAGCTTGCCGTAGAGGCGCCGCGCTACGCTATCCTCCAGATG
GGATGCGTAGAAGCCGCTGCCCGAAAGGATCATCGCGCACCACGCCGACGAGGCGTCGCC
GAGCGCGAGGTTCTCTATCACCTCGATTTGCGAGAACGGATCGGCCTCCGGACCGCCCCA
CGCCTTGGGCATGGCCATGCGAAACACTCCCGTCTTGTCCAGGGCGCTCAATACGCTACC
GGGGAGCTTGCGGGCTGCCTCGACCTCGGCTGCAGAAGCGCGCAGCGCGGGGACGAGCTC
TCGCGTTGAGGCGAGTATATCGTGAATGCTGAGATGGCATTGCGTGGAGCTCATTGGTCT
TCCTTGCGTTTTGTGGTCGTGCAGGCTCCGTCGCATCGAGCCGCGCGAGCTGCCTTTCGT
GCCCCCCTTTCATGACGAGCCTCTGCGAGGCAGAAACTCCGTGACTACGAGCGCCGCACG
ACCGCGGAACGCCATCGAGAGATGGCAGCAAACCTACAGCTGGGGGTGCGCGGGTGAACG
TATTTGCTTTTTTGTTTGATTGCAAGCGGATTCATTCGGCTGCCTAGGTTGGAATTCTCC
TTTCTTGATGTTTTCTAATGTATGGCGCGATGGACGCTCTGCTATCGGCGCCCTGGCGGG
GCGCGGATATATAACAAAAAGACGTGGCGAGGCGTATGTAAAATCACGCCTGCGCTGTGC
GGTGTGACGCCGGTTTTCGGATGTGCACCTTTATAAGGCCGTGCTTGATCGAAATGACTC
ATTGTGTTAAGCGTGGCGCCGATGGACAGAATGCTTGGGTTTCTGCGCGATGCATTGGCG
GATCTGCTGCAGGTTGCGCCGGGAGCCGTTCAGGATGATGTTCGATTCCACGAGCAGGGC
CTCGACTCCGCGAGGGCGCTGCTCCTGGTTGAAAAGCTGTCGAGCTGGACGGGGCGCGCT
CTCCCAGCCACGCTGGTATGGCAGCACAGCACCCTGAACGCGCTTGCTAGGCATCTGGCG
GATACAAGCGGGGAGCCCATTTCGACGGCCCTCGGCGCGCGCGCCATCGCGTCGTCGTCC
ATCGACGAGCCCGTCGCCATCGTCGGGCTCGGGTGCCGCATGCCTGGCGGCATCGCGTCC
CCCGAGGGGATGTGGCGGGCCCTGTGCTCGGGCGTGGATGCCATCGTCGAGGTTCCTCCG
GACCGCTGGGACGCTGAGGCCTGGCTGGACGCGGACGCCCAGGCGCCCGGGAAGATGACG
ACCCGATGGGGTGGTTTTCTCAATGAGATCGTGAGCTTCGACGCAGGGTTCTTCGGAATC
TCGCCGGCTGAAGCCAGGCAGATGGATCCGCAGCAGCGCTTCGCGCTCGAGACTGCCTGG
GCAGCGCTCGAGGACGCGCGCGTCGTTCCCACCGCGCTCGGGGGGTCTCGCACGGGCGTG
TTCTTCGGCGCCATGTGGCAGGAGTATGCCCGCGTCGCGGGGGCGGACGCGACAGGCATA
GAGCCACACTCCGCCGTGGGCTGGGATAACTCCATCATCCCCGCCCGCATCGCCTACGCT
CTGGGGCTTCGCGGGCCGGTGATGACGGTCAACACGGCGTGCAGCTCCTCGCTGGTAGCC
GTGCATCTCGCGGCGCTGAGCCTCAGGCGTGGGGAGGCGGATCTGGCGCTTGCGGGCGGG
GTCAACCTCATGCTGACCCCCCACGCCACGGTCCAGATGACCAAGTTCGGCGCGATGAGC
CCTGACGGCCGCTGCCGGGCTTTCGCCGCAGGAGCCAACGGCTACGTCCGTGGCGAGGGG
TGCGGCGTGGTGGTGCTGCGCCGCCTCTCGGACGCGCTCGTCGCAGGCGATCGCATCTAC
GCGGTGCTGCGAGGCAGCGCCGTGAACAGCGACGGCGCGTCCAATGGACTCACGGCACCG
AACCCTGAGGCCCAGGCTGACGTCATCCGAGACGCCTGGCAGCGAGCTGGGGTTGCGCCC
CGGGAGGTCGCGCATACGTCGAGACCCATGGTACGGGCGATCCTCGGCGATCCTATCGAG
GCCGCGGGGCTGGCCGCCGTGTTCGCGCCCGGCCGGGAGCAGCCGCTCCGCATCGGCTCG
GTAAAGACGAACTTCGGCCACCTCGAGGCTGCGGCGGGCATCGCCGGGCTCCTCAAGGCG
ACGCTCGCGTTGCATCACGGGCAGCTGCCGCCGAACCTGCATTTCGAGAGCCCGAACCCC
CACATCGACTTTCAGGCGCTCCGGCTCGAGGTTGTCGCACGGCAGCAAGGCTGGCCGAGG
GACGCGGGCCGTCTTGCCGGCGTCAGCAGCTTCGGCTTCGGCGGCACCAACGCCCATGTT
GCGCTGGAGGAGTCTCCCTACAGGCCGCAGATCCTCGTCCCCTTGGCTGCTGATGGGGAG
CAGGCGCTCCGCGATGCGGCGCGACGCCTCCTGGCCGATTTCGCTGCTTCGGGCGCGGGT
CGCGATGCGGAGCGGCTCGTGGTGCAGCCCGGCACGGGCTCCGTTCGCGCCATGGTCCAG
GGGCGCGATGGCGCCTCGGTGGAGGCTGCATTGCGGGAGCTGGCCGACGGTGGCGCGGAG
CTCCGCCCGATTTCCCGGCCGAGGCCGCGGCTGGTGTTCTTCTTTTCAGGACACGGCGGC
CACTGGGCGGGCATGGTCCGTGACCTGATGGCCGGGGAGCCGGTGTTCCGCGCGACCCTC
GAGGCCTGCGATCGAGCCGTCCGGCAGTTCACCGGGTGGTCCGTGATCGCCCAGCTCGCG
GCGAACGAGGCGTGCTCGCGGCTCGACCGAACCGACGTCGTGCAGCCCGTGGTCTTCAGC
GTTCAGGTCGCGCTTGCTCGCACCCTGCAAAGCTGGGGCGTGGTCCCTGAGCTCGTTTTC
GGCCAGAGCGTTGGCGAGGTCGCTGCGGCCGTGGTCGCGGGAGCCCTGTCGTTGCGCGAC
GGGGCGCGGGTGATCTCCACCTGGTCGGCTCTGATCGCGAATCATGCGTCAGGACGTGGC
GCGATGATCGTCGCCGATCTGTCGGTCGCGCAGGCCGAGCGCGTCCTCGCCAGCTGGGAC
GCGGGGGGCGTTCAGGTCGCCGTCCATCTGGCCCCCGGCCAGGTTTGCCTGGCCGGTCCG
GTCGATGCCGTCGAGCGTCTTGTGCGCCGGTTCGCGAGCGATGGCATCGGCACCATGCGC
GTGCGCATCGACTACGCCGCTCATACCGCCGAGATGGAGCCGTTGCTCCCCGAGATCGAG
CGGCAGCTCGCGGCGATCCAGCCCAGCGCGCCCACGATCCCGATGTGGTCCACGGTCGCG
AACCGGTATGTCGCCGGTCCGGAGCTCGATGGACACTACTGGGCGCGGAACCTCCGCGAG
CCGGTTCAGCTCGTCGAAGCTGTCGAGAGCGTGGCGCGGCAGCACAGCGCGTGCATCGTC
GAGGTGGGGCCGCATCCGGTCGCGGTGCGCTCGTTTGAAGCGTCGCTGAAGACGTGGGGA
GACAGCCGTTCTGTGGTCCTCGCGACCTGCTGGCGCGAGGTGGCCGCGCGGGGCGGGCTG
GAGGCGCTGCTCGGCGCGTTGTGGCGCGAAGGGATCGATGTCGACTGGGACGCGGTCCGC
ACGGGTGGCCGCGCGGCGACTCCGCGCGCGGTCACTCTCCCGCTGGTGGTGTCCGGGAAG
ACGGTGCAGGCCCTGCAGGAGGACGCCGCGCGCCTGGCGACACATCTTGATGGCCGAGGC
GACATGGCTCTCGTCGACGTCGCCTTCACCCTGGCGACCGCTCGCGCGCACTTCGCCACG
CGGGCGTCGGTGCAGGCGGCGAGCGTGTCGGAGGCGGTGGAGGGGCTGAGGGCGCTGTCG
GAGGGTCGAGCCGCGGCAGGTGTGGTGCGCGGGACGGGACTGCACGGGGGGAAGCTGGCG
GTGCTGTTCACGGGGCAGGGCAGCCAGCGGCTCGGGATGGGGAAGAGTCTTTACGAAGCG
TGCCCCGTGTTCCGTGCGGCGTTCGACGAGGTGTGCGAGGCGCTGGACGCGCATCTCGAC
CGTGGGTTGAGAGAGGTGGTGTTCGCGGAAGCGGGCAGCGAGCAGGAGGCGCTGCTGGAG
CGGACGGAGTACACGCAGCCCGGGTTGTTTGCGCTGGAAGTGGCGCTGTACCGTCAGTGG
GAGTCGTGGGGGCTGAAGCCCGCTGCGCTGCTGGGGCACTCGATAGGAGAGCTGAGCGCT
GCGCATGTGTCGGGCGTGCTGAGCCTTGCGGACGCTGCGAAGCTAGTGTGCGCCCGCGGT
CGGCTGATGCAGAGGTGCCAGGCGGGCGGAGCGATGATGTCGGTGGAGGCCTCGGAGCCG
GAGGTGCAGGGGGCGCTGTCGGCGATGGGGCTGGAGGGCGTGCTCGGGGTCGCGGGCATC
AACGGTCCGAGCCAGACGGTGCTGAGCGGGGACGAAGCGGCGGTGCTGGAGGTGGGGAGG
CGGTTCGAGGCGCAGGGCCGGCGCACGCGGCGTCTGCGCGTGTCGCACGCGTTCCACAGC
CCGCACATGGACGGGATGCTGGAAGAGTTCGGGAAGGTGGCGCGCGAGTGCGCGTATGGG
ATGCCGCAGGTGCCCGTGGTGAGCAGCGTGACGGGCGAGCTGTGGAGCGAGCAGGAGCTG
AGGTCACCCGAGTACTGGGTGAGGCAGGCGCGAGAGGCGGTGAGGTTCTTCGACGGGATG
CGCACGCTTGCGGCGGCGGGGGTGAGCACATACGTCGAGTGCGGTCCCGACGGCGTGCTG
TGCGCGCTGGGGGCGGGGTGCCTGCCGGAGGGAGCCGAGGCGACGTTTGTGGCGAGCCTG
CGGCGAGAGCAGGAGGAAGAGCGCGCGCTTGCAACGGCGGTGGCGACGGTGCACGTGCAA
GGGCACGAGGTGGACTGGGCCCAGGTGCTGTCGGGCCATGGCGGCCGGCCCGTGGAGCTG
CCGACGTACGCATTCCAGCGGCAGCGCTACTGGCTGGAGGCGCCGAGGGCGCGCGGTGAC
GTGGGCTCGGCGGGGCTGAAGGCGGCCGCCCATCCGCTGCTCGGCGCCGCGACGAAGCTC
GCCGACGGCGAGGGGCACCTGTTCACAGGGAGGCTGTCGCTGGCGGAGCATGCGTGGCTT
CGGGATCATCAGGTGTTTGGCAGGGTGGTGTTTCCGGGCACGGGGATGCTGGAGCTGGCG
CTGGCGGCGGGACGCGCGGTGGGCAGCCGGTCGCTGTCGGAGCTCACGCTCGCCGAGCCG
CTGGTGCTGGCCGAGGAGGGCGCGACGCGGCTGCAGCTGTCGGTCGGAGCGCCGGACACG
GCGGGCCGGCGCGAGGTGGGGCTGTACAGCCAGTCTGAGCAGGCCCCGGAGGACGCCCCG
TGGGTGCAGCACGCGACGGGGGTGTTGACGGACGAGACCCCCGGCGCTCCTGGCGAGCTC
GACGAGCTGTCGACGTGGCCTGTGCCGGGCGCGGAGGAGGTGGACCTGTCCGGGGTTTAC
GAGCGGCTGCGTGAGCGCGGGCTCGACTACGGTCCGGCGTTCCAGGGCCTCGTGGAGCTC
TGGCGTCGAGGCACGAGGCTTTACGGCCGGGTGGTGTTGCCCGAGAGCGCGATGGACAGC
GCCGAGGCGTATGGGGTGCACCCGGCGCTGATGGATGCCGCGCTGCATGCGCTGTTCGCG
GCCTCTTCGGAGGCGGCTGGAGCGGACATGGCGCTGCTGCCCTTCTCGTGGTCGGACGTG
GCGCTGCACGCGACGGGACCGCGCGAGCTCCGGGTGATGCTGGAGCTTGAAGGCGACATG
ACGCAAGCGGTGGCGTCGCTGTGGTTCGCGGACGCGGCGGGCCAGGCTGTGGCCAGGCTC
GGCGCCCTGCATCTGCGGCGAGCGACAGCGGAGCAGCTGGCAGTGGCCAGCCCTGCCAAT
GCCCAGCAGCTGTACCGGGTGGACTTCCAACCGGTGAGCGTGGTGGCGAGCGTCCCGGAG
GCGGGCTCGCTGGTGGTCGTCGGTCGACCGGAGGGAGGAGCGCGGCTGGCCGAAGCCCTG
GGGGCGGAGGCGATTGCCATCTCGATGCATTGGTTGCGCGCCTTCGAGCAGGGCGCGAGC
GCGCCTGAGCGGGTGGTGGTCGACGTCACCGCCGCCAGCCCCAGCCCGTTGGACGTGGCG
GTGTCGTCGCATGAGGCGACGAGGCAGGCGCTGTCGCTGCTGCAAGCGTGGCTGTCGGAG
CCACGGCTCGATGCTGTCGAGCTGGTGTGGGTGACGCGGGGCGCGGTGGGCGCGGCGCCG
GACGACGCCGTCCAGGATCTGGCGCGCGCGCCGCTGTGGGGGCTTGTTCGCGCGGCGCGC
AGCGAGCACCCCGAGCGCCGGCTGCGCTTGATCGATGTGGGGACCGAGCCCGTGGACGCT
GGGCTGCTGGCGCGGGCGCTGGCGATGGCGGCGGAGCCGGAGCTTGCGCTGCGCGGGGGC
GCTGCGCTGGCCGCGCGCCTGGTGCGCGTACAGGCGGCAGCGAAAGAGCTCACGCAAGCC
CGCGGGCTGGGCCCCGCGGGCACGGTGCTGGTGACCGGAGGCGTGGGCGGCCTGGGTCAG
ATCGTGGCGACGCACCTGGTGCGCGCGCACGGGGTGAGGCACCTTGTGCTGACGTCGCGC
CGGGGGCTGGAGGCGCCCGGGGCCCGCGAGCTTGTGCGGTCGCTCGCGGAGCTCGGCGCC
GAGACGGTGACGGTGGCTGCGTGCGACGTGTCGAAGCGGGAGGAGGTCGCGCGTGTGCTG
GCCGGCATCGACGCGGCGCGCCCGCTGAGCGCGGTGCTGCACCTGGCCGGCGTGCTCGAC
GACGGCGTGCTCGCCGCCCAGACGGCCGAGCGCCTCTCGCGGGTGCTGGCGCCCAAGGTG
GATGGGGCGCTGCACCTGCACGAGCTGACGCGGGAGCTGGATCTCGCGGCGTTCGTGCTG
TTCTCGTCGGTGGCCGGCACGTTTGGCACGGCGGGCCAGAGCAACTACGCGGCGGCGAAC
ACGTTCCTCGACGCGCTCGCGGCGCACCGGCGCGGCCGTGGGCTCGCGGGCGAGGCCGAG
CTGTCGCGCATCAGGCGCGCAGGATTTATCCCGATGTACGTAGCAGAGGGCTTGTTCCTG
CTGGACGTCGCGCTCTCGCGCTCTGAAGCCAGCCTGGTTCCGGCGCACTTCGATCTGGGG
AAGCTGCGATCGGGGCTTGACGCCAGCGGTGGAGTGCCCGCGCTGCTCCGGGCTCTCGTG
CGCCCGAGCCTGCGCAAGGCATCCTCCGGGACACAGGAGACCTCGGCGCTTCGGGCGCGC
CTCTCGGCGCTGCCGGAGTCGGCGCGGCTCGAAGCGCTGATCGACCTGGTGCGGGGCGAG
GTCGCCGCGGTGCTCGGGCTGCATCACCCCGCGTTGGTGGAGCCCCACCAGGTGCTGAAG
GAGGTTGGGCTCGACTCGTTGATGGCGGTGGAGCTGCGCAGTCGGCTGAGCTCCCGGGCC
GAGACTTCGCTGCCGGCGACCCTGGCCTTCGACCACCCGACGCCGCGGGCCATCGCAGAG
CTGCTCCTGAAGCAGGCCTTCTCGGAGCTACAGGAGACGGGGGGCGGCGCGCGTGTGCGC
CGTCGGGTGAGGGAGGACGAGCCGATCGCGATCGTGTCGATGGCGTGCCGGCTGCCGGGT
GGCATCGAGACGCCGGAGGATTTCTGGCGCGTCCTCCGTGAGGGGAAGGATGTCATCGAG
AGCTTCCCCTCCCGATATGAAGCTTCTTCTGTTTACGATCCCGATCCGGAGGCGGTGGGC
AAGAGCTACGCGCGCGAGGGGGGATTTCTACGGAACGTAGACCTGTTCGACGCGGGGTTC
TTCGGGATATCGCCGCGCGAAGCGCAGTCGATGGATCCCCAGCAGCGGCTGGTGCTGGAG
ACGGCGTGGGAGGCGCTGGAGCGGGCCGGCGTGCGCCCATCGGTGCTGAGCGAGAGCGCT
ACCGGGGTGTACCTGGGCTGGATGGGCTCGGACTACGCCGCTGGCCACGCGGACGACCTG
AGGACGCTCGATGGCTATCGCATCACGGGCGGGGCCGGCAGCGTCATCTCGGGCCGCGTG
GCGTATGTGCTTGGGCTGCAGGGCCCAGCGATCACGGTGGACACGGCGTGCTCGTCGTCG
CTGGTGTCGCTGCACCTCGCCTGCATGGCCCTGCGCCGGGGCGAATGCGACCTGGCGCTG
GCCGGCGGGGTGATGGTGATGACCACGCCCGCGACCTTCGTGGAGTTCAGCCGCCTCAAG
GGCATTGCGCGAGATGGCCGCTGCAAGAGCTTCTCTGCCCAAGCGGACGGCGTCATCTGG
GCCGAGGGGTGCGGGATGCTGGTGCTGAAGCGGCTGTCGGACGCGCGGCGCGACGGTGAC
CGTGTGCTTGCGGTGGTCCGTGGGTCAGCGGTGAACCAGGACGGTCGCAGCCAGGGCCTG
ACGGCGCCGAACGGCCCCGCGCAGCAGCGGGTGATCCAGCAGGCGCTGTCGTCGTGCGGG
CTGTCGCCCGAGGACATCGACGCGGTGGAGGCGCATGGTACGGGCACGAGCCTCGGAGAT
CCCATCGAGGCGGGAGCGCTTGCGGAGGTGTTCGGGCCTGGGCGCAAGGCCCAGCGGCCG
CTGTACCTGGGCTCGTCGAAGTCGAACCTCGGGCACGCGCAGTCGGCCGCCGGCGTGGCT
GGGGTGATCAAGATGGTGCTGTCGATGCAGCACGAGGTGCTGCCGCGGACGCTGCACGCG
GAGCAGCCGAGCCCGCACATTGGGTGGGAGGGGAGCGGGCTGTCGTTGCTGCAAGAGGCG
CGTCCGTGGCGGCGCAACGGCCGGGCGCGGCGCGCGGGCGTGTCGTCGTTCGGGATCAGC
GGGACGAACGCCCATGTCATCCTCGAAGAGGCGCCGGTGGAGGCGGTGCGCGAGCCGGTG
GCGGCGGAGGCGGCAGAGGGTGCAGCGATGCCGCTGTTGCTGTCGGGGCGAGACCAGGCC
TCGGTGGCGGCGCAGGCGGGGCGCTGGGCGAAGTGGCTGGAAGAGCACGCGGAGGTGGGG
TGGTCGGACGTGGTGQQGGACGGCAGCGCTGCACCGGACGCACTTCACCCGCGGGCGTCG
GTGCTTGCGGCGAGCGCTGCGGAAGCTGTGGAGGGGCTGAGGGCGCTGTCGGAGGGTCGA
GCAGCGGCGGGGGTGGTGCGCGGGACGGGAGTGCGCGGGGGGAAGCTTGCGGTGCTGTTC
ACGGGGCAGGGCAGCCAGCGGCTCGGGATGGGGCCGCGGCTTTACGAAGTGTACCCCGTG
TTCCGTGCGGCGTTCGACGAGGTGTGCGAGGCGCTGGACGCGCACCTCGCGCGTGGTCTG
CGGGAGGTGGTGTTCGCGGCTGCGGGCAGCGAGGAAGGAGCGTTGCTGGAGCGGACGGAG
TACACGCAGCCCGGGCTGTTTGCGCTGGAAGTGGCGCTGTACCGTCAGTGGGAGTCGTGG
GGGCTGAAGCCCGCTGCGCTGCTGGGGCACTCGATAGGAGAGCTGAGCGCTGCGCACGTG
GCGGGTGTGCTGAGCCTTGCGGACGCAGCGAAGCTAGTGTGCGCCCGCGGTCGGCTGATG
CAGGGGTGCGAGGCCGGGGGAGCGATGGTGTCGGTGGAGGCCTCGGAGCCGGAGGTGCAG
CGGGCGCTGTCGGAGGTCGGGGCGCAGGGGCGACTGAGCATCGCCGGGCTGAACGCGCCG
ATGCAGACGGTGCTGAGCGGGGACGAAGCGGCGGTGCTCGCGGTGGCGCGACGGCTGGAG
GCGCAGGGCCGGCGCACGCGGCGTCTGCGTGTGTCGCACGCGTTCCACAGCGCGCACATG
GACGGGATGCTGGAGGAGTTCGGGAAGGTGGCGCGGGAGTGCACGTACGCGCGGCCGCAG
CTGGCGGTGGTGAGCGGCGTGACGGGCGAGCTCGGTGGCGAAGAAGCGCTGATGTCGGCC
GAGTACTGGGTGAGGCAGGTGCGCGAGGCGGTGCGCTTCCTGGACGGGATGCGCACGCTT
GCGGCGGCGGGGGTGAGCACATACGTCGAGTGTGGGCCGGATGGCGTGCTGTGCGCGCTG
GGGGCGGGGTGCCTGCCGGAGGGAGCCGAGGCGACGTTTGTGGCGAGCCTGCGGCGAGAG
CAGGAGGAAGAGCGCGCGCTGGCGACGGCGGTGGCGACAGTGCACGTGCAGGGGCACGAG
GTGGACTGGGCCCGGGTGCTATCCGGTCAGGGCGGTCGTGCCGTGGAGCTGCCGACGTAC
GCATTCCAGCGGCAGCGCTACTGGCTGGAGGCGCCGAGGGCGCGCGGTGACGTGGGCTCG
GCGGGGCTGAAGGCGGCCGCCCATCCGCTGCTCGGCGCCGCGACGAAGCTCGCCGACGGC
GAGGGGCACCTGTTCACAGGGAGGCTGTCGCTGGCGGAGCATGCGTGGCTTCGGGATCAT
GGTGTGTTTGGCCAGGTGGTGTTTCCGGGCACGGGCATGCTGGAGGTTGCGCTGGCGGCT
GGGCGCGCGGTGGGCAGCCGGTCGCTGTCGGAGCTCACGCTCGCCGAGCCGCTGGTGCTG
GCCGAGGAGGGCGCGGCGCGGCTGCAGGTGATGATCGGAGCGCCAGATGCGGCGGGCCGG
CGCGAGGTCGGGCTGTACAGCCAGTCTGAGCAGGCCCCGGAGGACGCGCCGTGGGTGCAG
CACGCGACGGGGGTGTTGACGGACGAGCCCCCCGGCGCTCCTGGCGAGCTCGACGAGCTG
TCGACGTGGCCTGTGCCGGGCGCGGAGGAGGTGGACCTGTCCGGGGTTTACGGGCGGCTG
CGTGAGCGCGGGCTCGACTACGGTCCGACGTTCCAGGGCCTCGTGGAGCTCTGGCGTCGA
GGCACGAGGCTTTACGGCCGGGTGGTGTTGCCCGAGAGCGCGATGGACAGCGCCGAGGCG
TATGGGGTGCACCCGGCGCTGATGGATGCCGCGCTGCATGCGCTGTTCGCGGCCTCTTCG
GAGGCGGCTGGAGCGGACATGGCGTTGCTGCCCTTCTCGTGGTCGGACGTGGCGCTGCTC
GCGACGGGACCGCGTGAGCTCCGGGTGATGCTTGAGCTTGAAGGCGACATGACGCAAGCG
GTTGCGTCGCTGTGGGTCGCGGACGCGGCGGGCCAGGCTGTGGCCAGCGTCGGCGCCCTG
CATCTGCGGCGAGCGACAGCGGAGCAGCTGGCAGTGGCCAGCCGTGCCAATGCCCAGCAG
CTGTACCGGGTGGACTTCCAACCGGTGAGCGTGGTGGCGAGCGTCCCGGAGGCGGGCTCG
CTGGTGGTCGTCGGTGCAGCGGAGGGACGAGGGAGGCTGGCCGAAGCCCTGGGGGCGGAG
GCGATTGCCGATCTCGATGCATTGGTTGCGCGCCTCGAGCATGGCGCGAGCGCGCCTGAG
CGGGTGGTGGTCGACGTCACCGCCGCGAGCCCGAGCCCGTTGGACGTGGCGGTGTCGTCG
CATGAGGCGACGAGGCAGGCGCTGTCGCTGCTGCAAGCGTGGCTGTCGGAGCCGCGGCTC
GACGATGTCGAGCTTGTGTGGATGACGCGAGGCGCGGTGGGCGCGGCGCCGAACGACGCC
GTCGAGGACCTGGCGCACGCGCCGCTGTGGGGGCTTATTCGTACGGCGCGAAGCGAGCAC
CCCGAGCGCCGGCTGCGCTTGATCGATGTGGGGACCGAGCCCGTGGACGCTGGGCTGCTG
GCGCGGGCGCTGGCGACGGCGGCGGAGCCGGAGCTCGCGCTGCGCGGGGGCGCTGCGCTG
GCCGCGCGCCTGGTGCGCGTACAGGCGGCAGCGGAAGAGCTCACCCGAGGAGCCCGCGAG
CTGGACCCTGCGGGCACGGTGCTGGTGACCGGCGGGACAGGGGAGCTGGGGCAGGCGGTC
GCGGCGCACCTGGTGCGCGCGCACGGCGTGAGGCACCTGGTGCTGACGTCGCGGCGCGGG
CTGGAGGCGCCCGGGGCCCGCGAGCTTGTGCAATCGCTCGCGGAGCTCGGCGCCGAGACG
GTGACGGTGGCTGCGTGCGACGTGTCGAAGCGGGAGGAGGTCGCGCGCGTGCTGGCCGGC
ATCGACGCGGCGCGCCCGCTGAGCGCGGTGCTGCACCTGGCCGGCGTGCTCGACGACGGC
GTCCTGTCGTCGCAGACGCCGGAGCGCATTTCACGGGTGTTCGCGCCGAAGGTGGATGGG
GCGCTGCACCTGCACGAGCTGACGCGGGAGCTGGATCTCGCGGCGTTCGTGCTGTTCTCG
TCGGTGGCCGGCACGTTTGGCACGGCGGGCCAGAGCAACTACGCGGCGGCGAACACGTTC
CTCGACGCGCTCGCGGCGCACCGGCGCGGCGGCGGGCTCGCGGCGACGAGCCTGGCGTGG
GGCTTCTGGACACAGGCGGGCGTGGGGATGACAGCGCACCTGGGCAAGGCCGAGCTGTCG
CGCATGAGGCGCAACGGGTTCGTGCCGATGCCGGTGGAAGAGGGCCTCGCTCTGCTGGAC
GCCGCGCTTTTGCGCTCCGAAGCGAGCCTGGTCCCAGTGCACCTGGATCTCGCGCAGCTG
CAGCGTGGGCTGGAGTCCAGCGGCGAGCTGCCGGCGCTGTTTCGTGCGCTGTTGCGCCCC
AGCTTGCGCAAGGCGTCCACGGCAVCGGCAACGAGGCGAGACGCCTCGGCGCTGCGCGGG
CGCCTCTCGGCGCTGCCGGAGGCGGAGCGGCTGAATGCGCTCATCGAGCTGGTGCGGGGC
GAGGTCGCCGCGGTGCTCGGGCTCCAGCGCAGCGAGGCCGTGGGGGCAGAGCAGGTGCTG
AAGGGCCTCGGGCTCGACTCGCTGATGGCGGTGGAGCTGCGCAACCGCCTCGCCGCCCGG
GCGGAGACGTCCTTGCCGGCGACGCTGGTCTTCGACTACCCGACACCGCGAGCGATCGCA
GAGCTGCTGCTGAAGCAGGCGTTCTCGGGGCTGCAGGTGAAGGAAGCGCGGGCGCGTCGT
CATGCGGGGAAAGACGAGGCGGTGGCGATCGTATCGATGGCGTGCCGGCTGCCGGGAGGC
GTCGAGACGCCGGAAGACTACTGGCGTCTCTTGGCCGAGGGGAAAGACGCGATCGAGGGC
CTCCCTGCGCGCTGGGAGGCGCTTTCGGTCTACGACCCCGATCCGGAGGCGGTGGGCAAG
AGCTACGCGCGCGAGGGTGGATTCTTGCGGGACATCGACCTGTTCGACGCGGACTTCTTC
GGGATATCGCCGCGCGAGGCGCAGTCGATGGATCCCCAGCAGCGGCTGGTGCTGGAGACG
GCGTGGGAGGCATTGGAGCGTGCCGGCGTGCGCCCGTCGGCGCTGAGCGGGAGCGCCACC
GGAGTGTATCTCGGGTCGATGGGCTCGGACTACGGTGCTCTTCATACCGGCGGGCTGGAA
GCGCTGGACGGGTACCGGGGCACCGGGAGCGCGGCGAGCGTGCTCTCAGGCCGTGTGGCC
TACGTGCTCGGGTTGCAGGGCCCAGCGATCACGGTGGACACGGCGTGCTCGTCGTCGCTG
GTGTCGCTGCACCTCGCGTGCACGGCGTTGCGTCAGGGTGAATGCGACCTGGCGCTGGCC
GGCGGGGTGACGGTGATGAGCACCCCCGCGTTGTTCGTGGAGTTCAGCCGGCTCAAGGGG
ATGGCCCGCGACGGCCGCTGCAAGAGCTTCTCTGCGCGACTGACGGCGTCACCTGGGTCC
GAGGGGTGCGGGATGCTGGTGCTGAAGCGGCTGTCGGACGCGCGGCGCGACGGTGACCGT
GTGCTTGCGGTGGTCCGCGGGTCAGCGGTGAACCAGGACGGTCGCAGCCAGGGCCTGACG
GCGCCGAACGGCCCCGCGCAGCAGCGGGTGGTCCAGCGGGCGCTCTCGTCGTGCGGGCTG
TCGCCCGAGGACATCGACGCGGTGGAGGCGCACGGGACAGGCACGAGCCTCGGAGATCCG
ATCGAGGCGGGAGCGCTCGCGGAGGTGTTCGGGCCTGGGCGCAAGGCCGAGCGACCGCTG
TACCTGGGCTCGTCGAAGTCCAACCTGGGGCATACGGGGCCTGCGGCGGGTGTAGTCGGT
GTGCTCAAGATGGTGCTGTCGATGCAGCACGAGGTGCTGCCGCGGACGCTGCACGCGGAG
CAGCCGAGCCCGCACATTGGGTGGGAGGGGAGCGGGCTGTCGTTGCTGCAAGAGGCGCGT
CCGTGGCGGCGCAACGGCCGGGCGCGGCGCGCGGGCGTGTCGTCGTTCGGGATCAGCGGG
ACGAACGCGCATGTCATCCTCGAAGAGGCGCCGGTGGAGGCGGCGCGCGAGCCGGTGGAG
GCGGTGCGCGAGCCGTTGGCGACGGAGGGTGTTGCGATGCCGCTGTTGCTGTCGGGGCGA
GACGAGGCCTCGGTGGCGGCGCAGGCGGAGCGCTGGGCGAAGTGGCTGGAAGAGCACGCG
GAGGTGGGGTGGTCGGACGTGGTGAGGACAGCGGCGCTGCACCGGACGCACTTCGCCTCA
CGCGCATCGGTGCTTGCGGCGAGCGTGTCGGAGGCGGAGGAGGCGCTGCGGGCGCTGTCG
CAGGGTCGCGGCCACCGGGCGGTGTCGGCGGGCACGGCGCGTGCGCGAGGCAAGGTGGTG
TTCGTGTTCCCCGGCCAAGGGAGCCAGTGGCCGGGGATGGGCCGGGCGCTGCTGGAGCAG
AGCGCGGCGTTCGCGGAGGCGGTGCAGGCGTGCGATGAGGCGCTGCGGCCGTGGACGGGC
TGGTCTGTGCTGTCGGTGCTGCGCGGCGAGGCGGGTGAGGCAGGTGAGGAGCAGCCGTCG
CTGGAGCGGGTGGACGTGGTGCAGCCCGCGCTGTTCGCGATGTGCGTGGGTCTGGCCGCG
GCGTGGAGGTCGCTGGGGCTGGAGCCTGCGGCGGTGGTGGGCCACAGCCAGGGCGAGGTG
TCGGCGGCGGTGGTGTGCGGGGCGCTGTCGCTTGCGGAGGGAGCGCGGGTAGTGGCGCTG
CGCAGCCAGGCGGTGCGGCAGCGGTCGGGGATGGGGGCGATGATGCTGGTCGAGCGGCCG
GTGTCGGAGGTGCAGGAGCGCATCGCGCCGTACGGGGAGGCGCTTGCGATAGCGGCGGTG
AACACGTCGAGCTCGACGGTGGTGTCGGGTGACGTGGAGGCGGTGGACGGGCTGATGGGG
GAGCTGACGGCAGAAGGTGTGTTCTGCCGGAAGGTGAACGTCGACTACGCGTCGCACAGC
GCGCACATGGATGCGCTGCTGCCCGAGCTAGGAGCAAGCTGTCGTCGCTCAGGCCCGAAG
GCGACGCAGCTGCCGTTTTACTCGACGGTGGCAGGAGAGGTGTCGCGAGGCGAGGCGCTG
GACGGCGAGTACTGGTGCCGCAACCTTCGGCAGACGGTGCGCCTGGACCGAGCGCTGTCG
AAGCTGCTGGAGGACGGGCACGGTGTGTTCGTGGAGGTGAGCGCGCACCCGGTGCTGGCG
ATGCCGCTGACGACGGCGTGCGGGGAGGCGCAGGGGGTGGTGGTGGGGAGCCTGCAGCGC
GACGAAGGTGGGTTGTCGCAGCTGTACAGGACGCTGGGGCAGCTGCACGTGCAGGGGCAC
GAGGTGGACTGGGCACGGGTGCTGCCGGGCCATGGCGGTCGTGCCGTGGAGCTGCCGACG
TACGCATTCCAGCGGCAGCGCTACTGGCTGGAGGCGCCGAGGGCGCGCGGTGACGTGAGC
TCGGCGGGGCTGAAGGCGGCCGCCCATCCGCTGCTCGGCGCCGCGACGAAGCTCGCCGAC
GGCGAGGGGCACCTGTTCACAGGGAGGCTGTCGCTGGCGGAGCATGCGTGGCTTCGGGAT
CATGGTGTGTTTGGCCAGGTGGTGTTTCCGGGCACGGGCATGCTGGAGGTTGCGCTGGCG
GCTGGGCGCGCGGTGGGCAGCCGGTCGCTGTCGGAGCTCACGCTCGCCGAGCCGCTGGTG
CTGGCCGAGGACGGCGCGGCGCGGCTGCAGGTGATGATCGGAGCGCCAGATGCGGCGGGC
CGGCGCGAGGTGGGGCTGTAGAGCCAGCCTGAGCATGCCCCGGAGGACGCGCCCTGGGTG
CAGCACGCGACGGGAGTGTTGACGGACGAGCTCCCCGGCATCCCTGACGAGCTCGACGAG
CTGTCGATGTGGCCTGTGCCGGGCGCGGAGGAGGTGGACCTGTCCGGGTTTTACGAGCGG
CTGCGTGAGCGCGGGCTCCACTACGGTCCGAGCTTCCAGGGCCTCGTGGAGCTGTCGCGC
CAAGGCACCAGGCTTTATGGCCGGGTGGTGCTGCCAGGAACCGAGAAGGACAGGGAGGAG
GCGTATGGCTTGCATCCCGTCCTGATGGATGCAGCACTGCAGGTGCTTGGCGCAGCCGGC
GAGGGGCATTGGGAGGCGGATGCGTTGTTTATGCCCTTCTCCTGGGCAGACGCCGCAACG
CATGCCACGGGTCCGAGCGAGCTTCGGGTGCGCGTGGAGCTCGAAGAGACAGACGGCTCC
ACGCAGGCAACGGCATCTCTGTGCGCTGCAGATGCCGCAGGCCAGCCGGTGGCGAGCGTC
GGTGCTTTGCGTTTGCGCCGTGTGACGGCCGAGCAATTGAGGGCGGTCACCCGCACCGAT
GAGCAGCACCTGTACCGGGTGAGCTTCCAGCCCGTGAGCCTCGCGCAAGCCCCCCTGGAG
GCGGGCTCGCTGGTGGTCCTCGGTGCAGCGGAGGGACGAGGGCAGCTGGCCGACACCCTG
GGGGCGGAGGCGATTGCCGATCTCGATGCATTGCGCGCTTGGATCGAGCGGGGCGCGCCA
ACGCCTGTGCGGGTGGTGATCGACACGAACGCTGCCAGCTCACCGCGCTCGGACGTGGCG
GGGTCGTCGCACGAGGCGACGAGGCAGGCGCTGTCGCTGCTGCAAGCGTGGTTGTCGGAG
CCGCGGCTCGACGCTGTCGAGCTGGTGTGGGTGACGCGGGGCGCGGTCAGCGCAGCTCCG
GACGACGCCGTCGAGGACCTGGCGCACGGGCCGCTGTGGGGGCTTATTCGCACGGCGCGC
AGCGAGCACCCCGAGCGCCGGCTGCGCTTGATCGATGTGGGGACCGAGCCCGTGGACGCT
GGGCTGCTGGCGCGGGCGCTGGCGACGGCGGCGGAGCCGGAGCTTGCGCTGCGCGGGGGC
GCGGTGCTGGCCGCGCGGCTGGTGCGCGTACAGGCGGCAGCGGAGGAGCTCACCCGAGCC
CGCGGGCTGGACCCTGCGGGCACCGTGCTGGTGACCGGAGCCGTGGGCGGTCTGGGGCAG
GCGGTGACACGCCATCTGGTGCGCGCGCACGGGGTGAGGCACCTTGTGCTGACGTCGCGC
CGGGGGCTGGAGGCGCCCGGGGCCCGCGAGCTTGTGCAATCGCTCGAGGAGCTCGGCGCC
GAGACCGTGTCGATGGTGGCGTGCGACGTGTCGAAGCGGGAGGAGATCGCGCGCGTGCTG
GCCGGCATCGACGCGGCGCGCCCGCTGAGCGCGGTGCTGCACCTGGCTGGCGTGGTCCAT
GATGGCGTGATTCAGACGCAGACGGCCGAGCGCCTCGCGTGGGTGCTGGCGCCGAAGGTG
GACGGGGCGCTGCACCTGCACGAGCTGACGCGGGAGCTGGATCTCGCGGCGTTCGTGCTG
TTCTCGTCGGCGGCCGGTACGTTGGGCATGGCGGGCCAGGGCAATTACGCGGCGGCGAAT
ACGTTCCTCGACGCGTTCGCGGCGCACCGCCGCGCCCGCGGGCTCGCGGCGACGAGCCTC
GCCTGGGGTGTCTGGACACCGGCCGGTGGTGGCATGGCGGCACAGCTGGGGGCCGCGGAG
CTGGCACGGTTCAGTCGCTACGGAGTCGTGTCGATGTCCGTGGAAGAGGGGCTTTCGCTG
CTGGACGCCGCGCTGTCACGCCCTCAAGCGAGCCTGGTCCCTATGCACCTGGATCTCGCG
CAGCTGCAGCGTGGGCTGGAGGCCAACGGCGAGCTGCCGGCGCTGTTTCGTGCGCTGTTG
CGCCCCAGCTTGCGCAAGGCGTCCACGGCGACGAGGCGAGACGCCTCGGCGCTGCGCGGG
CGCCTCTCGGCGCTGCCGGAGGCGGAGCGGCTGAATGCGCTCATCGAGCTGGTGCGGGGC
GAGGTCGCCGCGGTGCTCGGGCTCCAGCGCAGCGAGGCCGTGGGGGCGGAGCAGGTGCTG
AAGGGCCTCGGGCTCGACTCGCTGATGGCGGTGGAGCTGCGCAACCGCCTCGCCGCCCGG
ACGGAGACGTCCTTGCCGGCGACGCTGGTCTTCGACTACCCGACGCCGCGGGCCATCGCG
GAGTTGCTCCTGAAGCTGGCCTTTTCGGGACCGCAGGTGATGGGAGCCCGCAGGGGGGTG
CGTCGTCATGCGGGGAAAGACGAGGCGGTGGCGATCGTATCGATGGCGTGCCGGCTGCCG
GGAGGCGTCGAGACGCCGGAAGACTACTGGCGTCTCTTGGCCGAGGGGAAAGACGTGATC
GAGGGCCTCCCTGCGCGCTGGGAGACGCTTTCGGTCTACGACCCCGACCCGGAGGCGGTG
GGCAAGAGCTACGCGCGCGAGGGTGGATTCTTGCGGGACATCGACCTGTTCGACGCGGAC
TTCTTCGGGATATCGCCCCGAGAGGCGCAGGCGATGGATCCCCACCAGCGGCTGGTGCTG
GAGACGGCATGGGAGGCATTGGAGCGTGCCGGCGTGCGCCCGTCGGCGCTGAGCGGGAGC
GCCACCGGGGTGTATCTGGGGGCCGCGGGTTCGGACTATGGCGCTTACCAGGGTGGCGGG
CTGGAGATGCTGGACGGGTACCGGGGCATCGGGAGCGCGGCGAGCGTGCTCTCAGGCCGT
GTGGCCTACGTGCTCGGGCTGCATGGCCCAGCGATGACGGTGGACACGGCGTGCTCGTCG
TCGCTGGTGTCGCTGCACCTCGCGTGCACGGCGTTGCGTCAGGGTGAATGCGACCTGGCG
CTGGCCGGCGGGGTGACGGTGATGAGCACCCCCGCGTTGTTCGTGGAGTTCAGCCGGCTC
AAGGGGATGGCCCGAGACGGCCGCTGCAAGAGCTTCTCTGGGCAGGCGGACGGCGCGGGC
TGGTCGGAGGGGTGCGGGATGCTGGTGCTGAAGCGGCTGTCGGACGCGCGGCGCGACGGT
GACCGTGTGCTTGCGGTGGTCCGTGGGTCAGCGGTGAACCAGGACGGTCGCAGCCAAGGC
CTGACGGCGCCGAACGGCCCCGCGCAGCAGCGGGTGATCCAGCAGGCGCTGTCGTCGTGC
GGGCTGTCGCCCGAGGACATCGACGCGGTGGAGGCGCATGGTACGGGCACGAGCCTCGGA
GATCCGATCGAGGCCGGAGCGCTCGCGGAAGTGTTCGGGCCTGGGCGCAAGGCCGAGCGA
CCGCTGTACCTTGGCTCGTCGAAGTCGAACCTTGGGCACGCGCAGGCTGCGCCGGGCGTA
GCCGGTGTGCTCAAGATGGTGCTGTCGATGCAGCACGAGGTGCTGCCGAACACGCTGCAC
GCGGAGCAGCCGAGCCCGCACATTGGGTGGGAGGGGAGCGGGCTGTCGTTGCTGCAAGAG
GCGCGTCCGTGGCGGCGCAACGGCCGGGCGCGGCGCGCGGGCGTGTCGTCGTTCGGGATC
AGCGGGACGAACGCCCATGTCATCCTCGAAGAGGCGCCGGTGGAGGCGGCTCGCGAGCCG
GTGGAGGCGGTGCGCGAGCCGGTGGAGGCGGAGGGTGTTGCGATACCGCTGTTGCTGTCG
GGGCGAGACGAGGCCTCGGTGGCGGCGCAGGCGGGGCGGTGGGCGAAGTGGCTGGAAGAG
CACGGGGAGGTGGGGTGGTCGGACGTGGTGAGGACGGCGGCGCTGCACCGGACGCACTTC
GAGTCGCGGGCGTCGGTGCTTGCGGCGAGCGCTGCGGGAGCTGTGGAGGGTCTTCGCGCG
CTGTCGTCGGGCGGCCGGATGCGGCGGTGGTGAGCGGGACGGCGAAGCGAGGCGGGAAAG
CTTGCGGTGCTGTTCACGGGGCAGGGCAGCCAGCGGCTCGGGATGGGGAAGAGGCTTTAC
GAAGTGTACCCCGTGTTCCGTGCGGCGTTCGACGAGGTGTGCCAGGCGCTGCACGCGCAT
CTCGACCGTGGGTTGAGAGAGGTGGTGTTCGCGGCCGCGGGCAGCGAGGAAGGAGCGTTG
CTGGAGCGGACGGAGTACACGCAGCCCGGGCTGTTTGCGCTGGAAGTGGCGCTGTACCGT
CAGTGGGAGTCGTGGGGGCTGAAGCCCGCTGCGCTGCTGGGGCACTCGATAGGAGAGCTG
AGCGCTGCGCACGTGGCGGGTGTGCTGAGCCTTGCGGACGCAGCGAAGCTAGTGTGCGCC
CGCGGTCGGCTGATGCAGGGGTGCGAGGCCGGGGGAGCGATGGTGTCGGTGGAGGCCTCG
GAGCCGGATGTGCAGCGGGCGCTGTCGGAGGTCGGGGCGCAGGGGCGACTGAGCATCGCC
GGGCTGAACGCGCCGATGCAGACGGTGCTGAGCGGGGACGAAGCGGCGGTGCTCGCGGTG
GCGCGACGGCTGGAGGCGCAGGGCCGGCGCACGCGGCGTCTGCGTGTGTCGCACGCGTTC
CACAGCGCGCACATGGACGGGATGCTGGAGGAGTTCGGGAAGGTGGCGCGGGGGTGCACG
TACGCGCGGCCACGGCTGGCGGTGGTGAGCGGCGTGACGGGCGAGCTCGGTGGCCAAGAA
GCGCTGATGTCGGCCGAGTACTGGGTGAGGCAGGTGCGCGAGGCGGTGCGCTTCCTGGAC
GGGATGCGCACGCTTGCGGCGGCGGGGGTGAGCACATACGTCGAGTGTGGGCCGGATGGC
GTGCTGTGCGCGCTGGGGGCGGGGTGCCTGCCGGAGGGAGCCGAGGCGACGTTTGTGACG
AGCCTGCGGCGAGAGCAGGAGGAAGAGCGCGCGCTTGCGACGGCGGTGGCGACAGTGCAC
GTGCAGGGGCACGAGGTGGACTGGGCCCAGGTGCTGTCGGGCCGTGGCGGCCGGCCCGTG
GAGCTGCCGACGTACGCGTTCCAGCGGCAGCGCTACTGGCTGGAGGCGCCCAAGACGACG
GCGGCACAGGCGAATGTCTCGTGGCCGGAGCGTGCGTTGTGGGACGCGGTGCAGAAAGGC
GAAGGCGTTGCGGATCTGCTGGAGCTGCCTGACGACGTGCGCGAGAGCGTCGCGCCGCTG
CTGCCGTACCTTGCGGCGTGGCGCCGGAGAAGGGACGCAGAAGCCACGGTGTCTGGCTGG
TTGTACGAGGAGGCGTGGCAAAGGGAAGCCAGCGCTGCCAGGGGCAAGCCGGACGTGAGG
GGCAGATGGCTGCTGGTGTCATCTCCGCGTGCCGGAGGGCTGACCGCGGCGGTGAGTGAT
GCGCTCGGAGCTGCGGGTGCAGAGGTGATCATCGAGCCGGCGACCGAAGAGCGAGCGCAG
CTGGCGGCGAGGTTGAGAGGGCTGGAGGGCGAGCTGCGTGGCGTCGTGGCGCTGAGCGCG
CCTGGGGAGCAAGGTGCGCTGGAGGAAGGGCGAGGGCCTCGCGGAGTGTACGAGGTGCTG
GCGCTGGCGCAGGCGCTCGGTGACGCTGGGCTCGATGCGCGGCTCTGGGTGTTGACGCAG
GGAGCGGTGAGCACGGAGGCAAGCGAAGGGGTGTCCGACCCTGCGCAGGCGCTGACGTGG
GGGTTGGGGCGGGTGGTGGGGCTGGAGCACCCCGAGCGCTGGGGTGGACTGGTAGACCTG
CCGGCGGAGGTGGACGCGGAAGCGGTGCAGCAGGTGCTGAGGACGCTCGTTGCCGAGGAC
CACGAGGACCAGGTGGCGGTGCGACGCGGTGGGCGTCTTGTGCGGCGCATCGTGCGGGTG
AGTGGAGAGGACGGCGGAGCGGGGTGGAAGCCGCGTGGCACGGTGCTCATCACGGGTGGA
GTGGGAGGGCTCGGGAGCCATCTGGCTCGCTGGTTGGCGGAGCGGGGAGCAGAGCACCTG
GTGCTGGCGTCACGCCGGGGCGCCGCGGCAGCGGGCGCGCGCGAGCTTCGGGAGGAGCTT
GAGGGGCGGGGCGCGCGCGTGACGCTTGCGGCGTGCGATGTGTCGGAGCGAGCGCAGGTC
GAGGCGCTGGTGAGGGAGCTTGAGCAGGACGAAGCGCCGCTGAGCGCGGTGGCGCATCTG
GCGGGGATAGTCCGCCGCGTGCCGGTGCGAGAGCTCGCGCCCGAGATGCTGGCGCAGGAG
CTCGCGGCGAAGGTCAACGGAGCATGGCACCTGCAGGAGCTGCTGGCAGAGCGCGAGCTG
GATGCGTTCGTGCTTTATGGCAGCATCGCTGGGCTGTGGGGCTCTGGGACGCAGGCCGGG
TACGGCGCGGCGAACGCAGGGCTCGACGCCCTCGCGCGCTACCGGCGTGCCCGAGGGCAG
ACGGCGACGGTGCTGCACTGGGGCCCGTGGTCCGGAGGCGGGATGGTGAGCGACGAGGCC
GAGCCGCAGCTCCGGAGCCGCGGGCTGGTGCCGATGTCGCCGGACAAGGCGCTTTGCGGG
CTCGAGGTTGGGCTGCGGCGCACGTCGGTGGCGATCGCGGACGTGGACTGGTCGCGCTTC
GCGCCGCTGTTCTGCGCGGCGCGGCCGAGGCCGCTGCTGTACGGGATCGAGCAAGCGCGC
CATGCGCTGGAGGGCCGGACACCGCAGCAGGCCGCGGGCGGAGCGGGGGACAAGGCGCTG
CGGGAGATGCTGCTCGGCCTGCCGGCTGTGGAGCGGAGCGAGCGGCTGCGCGAGCTCGTG
GCGAGCGAGACGGCGGCGGTGCTGCGCGTGAAGGATCCGAGCGGCCTGGACCCGGAGCGA
GGCTTTCTGGACCTCGGGCTGGACTCGTTGATGGCGGTGGAGCTGTCGAAGCGGCTGCAG
CAGCGGACGGGGGTGTCGGTCACAAGGACGTTGATCTTCGATTATCCGACGCAAGGCGAG
GTAACGCGCTGGCTGCTGGAGCAGCTGATGCCGCCGGAGCGACCGGCGGCGGACGAGCAC
GGCGTGAGCCGTGGACCGGAGCGGAGCGCGCCGATAGCGATCGTGGGCGTGGGGCTGCGC
ATGCCGGGCGGAGCGAACGATCTGGAGAGCTTCTGGCAAGTGCTCGTGGAGGGGCGGGAT
ACGCTAAGGCCGATCCCGACCGACCGTTTCGACGTGGAGGCGATGTACGATCCTAACCCC
GAGGCCAAGGGCAAGACGTACGTGAAGCATGCCTCGCTGCTGGACGACGTGGCATCGTTT
GACGCGGGGTTCTTCGGGATAAGCCCGCGCGAGGCGGAGCCGATGGATCCGCAGCACCGG
CTGCTGCTGGAGACTGCGTGGAGCGCGCTGGAGGACGCGGGAGTGCGTCCAGACCAGCTG
AAGGGCTCGGACACGGGTGTGTTCGTGGGCGTGGCGCCGAGCGAGTATGCGAGCTATCGC
GGCAAGAGCGCGAACGAAGATGCGTATGCGCTGACGGGGACCGCGCTGAGCTTCGCAGCG
GGCCGTGTGGCATATCATCTCGGGCTGCAAGGCCCTGCGGTGTCGGTCGATACGGCGTGC
AGCTCGTCGCTGGTGGCGCTGCACCTGGCGTGCGACGCATTGAGGCGCGGCGATTGCGAG
GTGGCTCTGGCGGCCGGCGTGCAGGTGCTCGCGAACCCGGCGGGGTTTGTGCTGCTGTCG
CGCACGCGTGCGGTCTCGCCCGACGGACGGTGCAAGACGTTCTCGCAGGCGGCCGACGGC
TACGGCCGCGGCGAGGGCGTCGGGGTGGTGGTGCTGATGCGTCTTTCGGACGCACAGGCG
CAGGGGATGCGGGTGCTGGGCGTGGTGCGGGGCACGGCGGTCAATCAGGACGGCGCGAGC
AGCGGGATCACGGCGCCGAACGGCACGGCCCAGCAGAAGGTGGTGCGCGCGGCGCTGCGG
AACGCGGGGCTGGAGGCGTCGAGCATCGATGTGGTCGAGTGCCACGGTACGGGCACGTCG
CTGGGCGATCCGATCGAGGTGCAAGCGCTCGGCGCGGTGTACGGGCAAGGCAGGGAGGCG
ACTCGCCCGCTGCGGCTTGGAGCGGTCAAGAGCAACATCGGTCACCTGGAGTCGGCCGCC
GGCATCGCCGGAGTGTGCAAGATCCTGGCGGCGTTTCGGCATGAGGCCCTGCCGGCGACG
TTGCACAGCTCGCCGCGCAACCCCCAGATCTCCTGGGAGAGTCTGCCGGTGCAGGTGGTC
GACCGCCTGACCGGCTGGCCTCGGCGCGCCGACGGCCTCCCCCGCTTTGCGGGCGTGTCG
TCGTTTGGCATCAGCGGGACGAACGCGCATGTCATCCTCGAAGAGGCGCCGCTTGAGGCG
GTGCGCGAGCCGGCGGCGGTGCGCGAGCCGTTGGCGGCGGAGGGTGTCGCGATCCCGCTG
TTGCTGTCGGGGCGAGACGAGGCCTCGGTGGGGGCGCAGGCGGAGCGCTGGGCGAAGTGG
CTCGGAGAGCACGCGGAGGTGCGGTGGCCGGACGTGGTGAGAACGGCGGCGCTGCACCGG
ACGCACTTCGCCTGGCGCGCATCGGTGCAGGCGGCGAGCGTGTCGGAGGCGGTGGAGGGG
CTGAGGGCGCTGTCGGAGGGTCGAGCCGCGGCAGGTGTGGTGCGCGGGACGGGAGGGCGC
GGGGGGAAGCTTGCGGTGCTGTTCACGGGGCAGGGGAGCCAGCGGCTCGGGATGGGGAAG
AGACTTTACGAAGTGTACCCCGTGTTCCGTGCGGCGTTCGACGAGGTGTGCGAGGCGCTG
GACGCGCATCTCGACCGTGGGTTGAGAGAGGTGGTGTTCGCGGAAGCGGGCAGCGAGCAG
GAGGCGCTGCTGGAGCGGACGGAGTACACGCAGCCCGGGTTGTTTGCGCTGGAAGTGGCG
CTGTACCGGCAGTGGGAGGCGTGGGGAGTGAGGCCCGCGGCGCTGCTGGGGCACTCGATA
GGAGAGCTGAGCGCTGCGCACGTGGCGGGCGTGCTGAGCCTTGCGGACGCAGCGAAGCTA
GTGTGCGCCCGCGGTCGGCTGATGCAGAGGTGCCAGGCGGGCGGAGCGATGATGTCGGTG
GAAGCGTCGGAGCCGGAGGTGCAGGGGGCGCTGTCGGCGATGGGGCTGGAGGGCCGGCTT
GGGATCGCGGGCATCAACGGTCCGAGCCAGACGGTGCTGAGCGGGGACGAAGCGGCGGTG
CTGGAGGTGGGCAGGCGGTTCGAGGCGCAGGGCCGGCGCACGCGGCGTCTGCGCGTGTCG
CACGCATTCCACAGCGCGCACATGGACGGGATGCTGGAAGAGTACGGGAGGGTGGCGCGG
GAGTGCGCGTATGGGAGGCCGCAGGTACCCGTGGTGAGCGGCGTGACGGGCGAGCTCGGT
GGCGAAGAATCGCTGATGTCGGCCGAGTACTGGGTGAGGCAGCTGCGCGAGGCGGTGCGC
TTCCTGGACGGGATGCGCACGCTTGCGGCGGCGGGGGTGAGCACATACGTCGAGTGCGGT
CCGGATGGCGTGCTGTGCGCGCTGGGGGCGGGGTGCCTGCCGGAGGGAGCCGAGGCGACG
TTTGTGGCGAGCCTGCGGCGAGAGCAGGAGGAAGAGCGCGCGCTTGTGACGGCGGTGGCG
ACGGTGCACGTGCAAGGGCACGAGGTGGACTGGGCCCAGGTGCTGTCGGGCCATGGCGGC
CGGCCCGTGGAGCTGCCGACGTACGCGTTCCAGCGGCAGCGCTACTGGCTGGAGGCGCCG
AGGGCGCGCGGCGACGTGGGCTCGGCGGGGCTGAAGGCGGCCGCCCATCCGCTGCTCGGC
GCCGCGACGAAGCTCGCCGACGGCGAGGGGCACCTGTTCACAGGGAGGCTGTCGCTGGCG
GAGCATGCGTGGCTTCGGGATCATCAGGTGTTTGGCAAGGTGGTGTTTCCGGGCACGGGG
ATGCTGGAGCTGGCGCTGGCGGCGGGGCGCGCGGTGGGCAGCCGGACGCTGTCGGAGCTG
GTTCTGGCCGAGCCGCTGGTGCTGGCCGAGGAGGCCGCGGCGCGGCTGCAGCTGTCGGTC
GGAGCGCCGGACGCGGCGGGCCGGCGCGAGGTAGGGCTGTACAGCCAGTCCGAGCAGGCG
CCGGAGGACGCGCCGTGGGTGCAGCACGCCACGGGCGTGTTGACGGACGAGATCCCCGGC
GCCCCCGGCGAGCTCGACGAGCTGTCGACGTGGCCTGTGCCGGGCGCGGAGGAGGTGGAC
CTGTCCGGGTTTTACGAGCGGCTGCGTGAGGGCGGGCTCGACTACGGTCCGGTGTTCCAG
GGCCTCGTGGAGCTCTGGCGTCGAGGCGCGAGGCTTTACGGCCGGGTGGTGTTGCCCGGG
AGCGCGAGGGGCAGCGCCGAGGCGTATGGGGTGCATCCGGCGCTGATGGACGCCGCGCTC
CACACGATGGTCGCAGCTTTCTCTCAGATGTCAGGGCCAGACGGCGTGTTGTTGCCGTTC
GCCTGGTCGGACGTGGCGCCGCACGCGACGGGGGCGAGCGAGCTTCGGATCCGAGTGGAG
ATGCAGGAACAAAGCGCACAGCAGCCAGCGGCTTCGCTGTACGTCGCAGACTGCACGGGG
CAGGTCGTGGCGAGCATCGGCGCTCTACGTCTGCGCCGGGCGACGGCCGAGCAGCTGCGG
ACCGCCGTTCACGCTGGTGGCCAACATATGTATCAGGTGAGCTTCCAGCCTGTGGACCTC
GCAGCACCTCCCTTGGTGACGGGCTCGCTGGTGGTCATCGGTGCACCGAAGGGAGGAGCG
CGGCTGGCCGAAGCCCTGGGGGCGGAGGCGATTGCCGATCTCGATGCATTGGTTGTGCGC
CTCGAGCATGGCGCGAGCGCGCCTGAGCGGGTGGTGGTCGACGTCACCGCCGCGAGCCCG
AGCCCGTTGGACGTGGCGGGGTCGTCGCATGAGGCGACGAGGCAGGCGCTGTCGCTGCTG
CAAGCGTGGCTGTCGGAGCCGCGGCTCGAAGCGACCGAGCTGGTGTGGATCACGCGGGGC
GCGGTGGGCGCGGCGCCAGACCACGCCGTCGAGGACCTGGCGCGCGCGCCGCTGTGGGGG
CTTGTCCGCGCGGCGCGAAGCGAGCACCCCGAACGCGGGCTGCGCTTGATGGATGTGGGG
ACCGAGCCCGTGGACGCTGGGCTGCTGGCGCGGGCGCTGGCGACGGCGGCGGAGCCGGAG
CTTGCGCTGCGCGGGGGCGCTGCGCTGGCCGCGCGCCTGGTGCGCGCACAGGCGGTAGCG
GAAGAGCTCACCCGAGCCCGCGAGCTGGACCCTGCGGGCACGGTGCTGGTGACCGGCGGG
ACAGGGGAGCTGGGTCAGGCGGTCGCGGCGCACCTGGTGCGCGCGCACGGGGTGCGGCAC
CTTGTGCTGACGTCGCGGCGCGGGCTGGAGGCGCCCGGGGCCCGCGAGCTTGTGGAATCG
CTCGCGGAGCTCGGCGCCGAGACGGTGACGGTGGCCGCGTGCGACGTGTCGAAGCGGGAG
GAGGTCGCGCGTGTGCTGGCCGGCATCGACGCGGCGCGCCCGCTGAGCGCGGTGCTGCAC
CTGGCCGGCGCGCTCGACGACGGCGTGCTCGCCGGCCAGACGGCCGAGCGCCTCTCGCGG
GTGCTGGCGCCGAAGGTGGACGGGGCGCTGCACCTGCACGAGCTGACGCGGGAGCTGGAT
CTCGTGGCGTTCGTGCTGTTCTCGTCGGTGGCCGGTACGTTTGGCACGGCGGGCCAGAGC
AACTACGCGGCGGCGAATACGTTCCTCGACGCGCTCGCGGCGCACCGGCGCGGCTGCGGG
CTCGCGGCGACGAGCCTGGCGTGGGGGTTGTGGGCGCAAGCGGGCGTGGGGATGACAGCG
CACCTGGGCGAGGCCGAACTGTCGCGCATCAGGCGCGCAGGGCTCGTGCCGATATCGGTC
GACGAGGGCCTCGCTCTGCTGGACGCCGCGCTCTCACGCTCTGAAGCGAGCCTGGTCCCA
GTGCACCTGGATCTCGCGCAGCTGCAGCGTGGGCTGGAGTCCAGCGGCGAGCTGCCGGCG
CTGCTTCGCGCGCTGGTGCGCCCCGGGCTGCGCAAGGCGTCCTCTGCCGCGAGGAAGGAG
GCGTCGACGCTCCGCGAGCGCCTCTCGGCGCTGCCGGAGGCGGAGCGCCTGAGCTCGCTC
ATCGACCTTGTGCGGGCCGAGGTCGCCGCGGTGCTCGGGCTTCAGCGCGGTGACGCGATT
CCTACGGCCCAGCCCTTGAGGGAGCTCGGAATGGATTCGCTCATGGCCGTCGAAGTCCGC
AATCGGCTCGCCTTGCTGGTCGGAAGCAACTTGCCTGCCACTTTGCTTTTCGACCATCCA
TCTGCCACGCACATCGCGAAGTTCCTCCTGTCAAAGTTCGGAAACGGTGAGCGCCGGAAT
CTGCTGCGTACAGCGGACTCCATGTCCGACGAGGAAATTCGCGCGTTCATGCTCAGCCTC
TCCGTCAGTCTCGTGCGTCGTTCAGGCCTCCTCCCCAAGCTCTTGGAGCTGCGGGGGCCG
TCCGAAACATCCGTCGAGGTTCCGGTTCCCATTTCCGATTTCGAAGATCTCGCCGACGAG
CAGCTGGCCTTGCAAGCCTTGCAAATGATTTCGAACTCAGAGGATCTCCATGAATAGCAG
CGCCGCCTCTCCTACGCTTCGTGAGGCGTTGACCCGTGCATTGAAAGAGTTGCAGAGGCT
GCAGGCCAGCCACTCGGATCTCCGTTCAGGGCCCATCGCCATCGTATCGATGGCGTGCCG
GCTGCCGGGGGGCGTCGCTACGCCGGAAGACTACTGGCGTCTCCTGGAGGAGGGGAGAGA
TGCGATCGAGGCCTTCCCTGCGCGCTGGGATGCACCTTCGATTTACGACCCCGATCCGGA
GGCGGTGGGCAAGACCTACGTGCGCGAGGGTGGATTCCTGCGGGACATCGACCTGTTCGA
TGCGGGTTTCTTCGGGATATCGCCCCGAGAGGCGCAGGCGATGGATCCCCAGCAGCGGCT
GGTGCTGGAGACGGCGTGGGAGGCACTGGAGCGGGCTGGCGTGCGCCCGTCGGCGCTGAG
CGAGAGCTCCACCGGGGTGTACCTGGGCTCGATGGGCTCGGACTACGGTGCTCTTTACGG
CAGCGACCTGGCGGCGTTGGACGGCTACCGGGGCACCGGGAGCGCGGCGAGCGTGCTCTC
AGGCCGTGTGGCGTACGTGCTCGGGCTGCAGGGCCCAGCGATCACGGTGGACACGGCGTG
CTCGTCGTCGCTGGTGTCGCTGCACCTGGCGTGCACGGCCCTGCGTCAGGGCGAGTGCGA
TCTGGCCTTGACCGGTGGGGTGATGGTGATGACCACACCCGCGGGATTCGTGGAGTTCAG
CCGCCTCAAGGCCCTTGCACGGGACGGTCGTTGCAAGAGCTTCTCTGCGCGAGCTGACGG
CGTCATCTGGTCCGAGGGGTGCGGGATGCTGGTGCTGAAGCGGCTGTCGGACGCGPGGCG
CGACGGTGACCGTGTGCTGGCGGTGATCCGTGGGTCAGCGGTGAACCAGGACGGTCGCAG
CCAGGGTTTGACGGCGCCGAACGGCCCTGCCCAGCAGCGGGTGATCCAACAGGCGCTCTC
GTCGTGCCGGCTGTCGCCCGAGGACATCGACGCGGTGGAGGCGCATGGGACGGGCACGAA
CCTGGGCGACCCGATCGAGGCCGGAGCGCTCGTGGAGGTGTTCGGGCCTGGGCGCAAGGC
CGAGCGGCCGCTGTACCTTGGCTCGTCGAAGTCGAACCTGGGACACGCGGGGCCTGCGGC
GGGCGTCGCCGGTGTGCTCAACATGGTGCTGTCGATGCAGCACGAGGTGCTGCCGCGGAC
GCTGCACGCGGAGCAGCCGAGCCCGCACATTGGGTGGGAGGGGAGCGGGCTGTCGTTGCT
GCAAGAGGCGCGTCCGTGGCGGCGCAACGGCCGGGCGCGGCGCGCGGGCGTGTCGTCGTT
CGGGATCAGCGGGACGAACGCGCATGTCATCCTCGAAGAGGCGCCGGTGGAGGCGGCGCG
CGAGCCGGTGGAGGCAATGCGCGAGCCGTTGGCGACGGAGGGTGTTGCGATGCCGCTGTT
GCTGTCGGGGCGAGACGAGGCCTCGGTGGGGGCGCAGGCGGAGCGCTGGGCGAAGTGGCT
CGGAGAGCACGGGGAGGTGCAGTGGTCGGACGTGGTGAGGACAGCGGCGCTGCACCGGAC
GCACTTCGCCTCACGCGCATCGGTGCTTGCGGCGAGCGTGTCCGAGGCGGAGGAGGCGCT
GCGGGCGCTGTCGCAGGGTCGCGGCCACCGGGCGGTGTCGGCGGGTACGGCGCGTGCGCG
AGGCAAGGTGGTGTTCGTGTTCCCCGGCCAAGGGAGCCAGTGGCCGGGGATGGGCCGGGC
GCTGCTGGAGCAGAGCGCGGCGTTCGCGGAGGCGGTGCAGGCGTGCGATGAGGCGCTGCG
GCCGTGGACGGGCTGGTCTGTGCTGTCGGTGCTGCGCGGAGATGGCGGGGAGGAGCAGCC
GTCGCTGGAGCGGGTGGACGTGGTGCAGCCCGCGCTGTTCGCGATGTGCGTGGGTCTGGC
CGCGGCGTGGCGGTCGCTGGGGCTGGAGCCTGCGGCGGTGGTGGGCCACAGCCAGGGCGA
GGTGTCGGCGGCGGTGGTGTGCGGAGCGCTGTCGCTTGCGGAGGGAGCGCGGGTAGTGGC
GCTGCGCAGCCAGGCGGTGCGGCAGCGGTCGGGGATGGGGGCGATGATGCTGGTCGAGCG
GCCGGTGTCGGAGGTGCAGGAGCGCATCGCGCCGTACGGGGAGGCGCTTGCGATAGCGGC
GGTGAACACGTCGAGCTCGACGGTGGTGTCGGGTGACGTGGAGGCGGTGGACGGGCTGAT
GGTGGAGCTGACGGCAGAAGGTGTGTTCTGCCGGAAGGTGAACGTCGACTACGCGTCGCA
CAGCGCGCACATGGATGCGCTGCTGCCCGAGCTAGGAGCGAAGCTGTCGTCGCTCAGGCC
GAAGGCGACGCAGCTGCCGTTTTACTCGACGGTGACAGGAGAGGTGTCGCGGGGCGAGGC
GCTGGACGGCGAGTACTGGTGCCGCAACCTTCGGCGGACGGTGCGCCTGGACCGAGCGCT
GTCGAAGCTGCTGGAGGACGGGCACGGTGTGTTCGTGGAGGTGAGCGCGCACCCGGTGCT
GGCGATGCCGCTGACGACGGCGTGCGGGGAGGCGCAGGGGGTGGTGGTGGGGAGCCTGCA
GCGCGACGAAAGTGGGTTGTCGCAGCTGTACAGGACGCTGGGGCAGCTGCACGTGCAGGG
GCACGAGGTGGACTGGACACGGGTGCTGTCGGGCCACGGCGGTCGTGTCGTGGAGCTGCC
GACGTACGCGTTTCAGCGGCAGCGTTACTGGCTGGATATCTCGAAGGCGCGTAGCGACGT
GAGCTCGGCGGGGCTCAAGGCAGCAGCACACCCGCTGCTCGGCGCCGCGACGAGGCTCGC
CGACGGCGAGGGGCACCTGTTCACAGGGCGGCTGTCGCTGGCGGAGCATCCGTGGCTTCG
GGATCATGAGGTGTTTGGCCAGGTGGTGCTTCCGGGCACGGGGACGCTGGAGCTGGTGCT
GGCGGCGGGGCGCGCGGTGGGCAGCCGGTCGCTGTCGGAGCTCACGCTGGCCGAGCCGCT
GGTGCTGGCCGAGGGCGCGGCGCGGCTGCAGGTGATGATCGGAGCGCCGGACGCGGCGGG
CCGGCGCGAGGTGGGGCTGTACAGCCAGCCTGAGCAGGCCCCGGAGGACGCGCCGTGGGT
GCAGCACGCGACGGGAGTGTTGACGGACGAGCCCCCCGGCATCCCTGTCGAGCTCGACGA
CCTGTCGACGTGGCCTGTGCCGGGCGCGGAGGAGGTGGACCTGTCCGGGCTTTACGAGCG
GCTGCGTGAGCGCGGGCTCCACTACGGCCCGGCGTTTCAAGGGCTCGTGGAGCTGTCGCG
CCAAGGCACAACCTACTTCGGTCGGGTGGTGCTGCCGGGGACCGAGAAGGACAGGGCGGA
GGCGTATGGCGTGCACCCGGCGTTGATGGACGCCGCGCTCCACACGATGGTCGCGGCCTT
CTCCGAGAGCCCAGGGGCGAACGAGGTGCTCGTGCCGTTTGCCTGGTCGGACGTGGCGCT
GCACGCGACGGGGGCGAGCGAGCTTCGGGTCCGGGTAGAGCTCCAGGACGGAGGCGCACA
CCAGGACACCGCTTCGCTGCAAGTCGCAGACTCCACGGGGCAGGCCGTGGCGAGCATCGG
CGCTCTACATCTGCGCCGGGCGACGGCCGAGCAGCTGCGGACCGCCGTTCACGCTGGTGG
CCAACATATGTATCAGGTGAGCTTCCAGCCTGTGGAGCTCGCGGCAGCCCCCCTGGAGGC
GGGCTCGCTGGTGGTCGTCGGTGCAGCGGAGGGACGAGGCAGGCTGGCCGAAGCCCTGAG
GGCCGAGGCGATTGCCGATCTCGAAGCATTGGTTGCGCGCCTCGAGCAGGGCGCGAGCGC
GCCTGCGCGGGTGGCGGTCGACACGACAGCTTTAGGACAGAGTCAGTCGGGAGTGGCGTC
GTTGTCCCACGAGGCGACGAGGCAGGCGCTGTCGCTGCTGCAAGCGTGGCTGTCGGAGCC
GCGGCTCGACGCTGTCGAGCTGGTGTGGGTGACGCGGGGCGCGGTGGGCGCGGCGCCGGA
CGACCCCGTCCAGGATCTGGCGCGCGCGCCGCTGTGGGGGCTTGTTCGCGCGGCGCGCAG
CGAGCACCCCGAGCGCCGGCTGCGCTTGATCGATGTGGGGACCGAGCCCGTGGACGCTGG
GCTGCTGGCGCGGGCGCTGGCGACGGCGGCGGAGCCGGAGCTTGCGCTGCGCGGGGGCGC
TGCGCTGGCCGCGCGCCTGGTGCGCGCGCAGGCGGCAGCGGAAGAGCTCACCCGAGGAGC
CCGCGAGCTGGACCCTGCGGGCACGGTGCTGGTGACCGGCGGGACAGGGGAGCTGGGTCA
GGCGATCGCGGCGCACCTGGTGCGCGCGCACGGGGTGAGGCACCTTGTGCTGACGTCGCG
TCGCGGGCTGGAGGCGCCCGGGGCCCGCGAGCTCGTGCAGTCACTCGAGGAGCTCGGCGC
CGAGACGGTGACGGTGGCTGCGTGCGACGTGTCGAAGCGGGAGGAGGTCGCGCGTGTGCT
GGCCGGCATCCACGCGGCGCGTCCGCTGAGCGCGGTGCTGCACCTGGCCGGCGTGCTCGA
CGACGGCGTGCTCACCGCCCAGACGGCCGAGCGCCTCTCGCGGGTGCTGGCGCCGAAGGT
GGACGGGGCGCTGCACCTGCACGAGCTGACGCGGGAGCTGGATCTCGCGGCGTTCGTGCT
GTTCTCGTCGGCGGCCGGTACGTTTGGCGCGGCGGGCCAGAGCAACTACGCGGCGGCGAA
CACGTTCCTCGACGCGCTCGCGGCGCACCGGCGCGGCGGCGGGCTCGCGGCGACGAGCCT
GGCGTGGGGCTTCTGGACACAGGCGGGCGTGGGGATGACAGCGCACCTCGGCGAGGCCGA
GCTGTCGCGCATGAGGCGCAATGGGTTCGTGCCGATGCCGGTGGAAGAGGGCCTCGCTCT
GCTGGACGCCGCGCTCTCACGCCCTGAAGCGAGCCTGGTCCCAGTGCACCTGGATCTCGC
GCAGCTGCAGCGTGGGCTGGAGTCCAGCGGCGAGCTGCCGGCGCTGTTTCGTGCGCTGTT
GCGCCCGAGCTTGCGCAAGGCGTCCTCGGCAACGAGGCGAGACGCCTCGGCGCTCCGCGA
GCGCCTCTCGGCGCTGCCGGAGGCGGAGCGGCTGAATGCGCTCGTCGAGCTGGTGCGGGG
CGAGGTTGCGGCCGTGGCAGGGCTTCAGCGCGGCGAGGCTGTGGCAGCGGATCAGGTGCT
CAAGGAGCTGGGGCTCGACTCGCTGATGGCGGTGGCGCTGCGCAACCGCCTCACGTCCCG
TACCGAGACGTCCTTGCCGGCGACGCTGGTCTTTGACTACCCGACGCCGCGGGCGATCGC
AGAGCTGCTGCTGAAGCAGGCGTTCTCGGGGCTGCAGGTGAAGGAAGCGCGGGCGCGGGT
GCGTCGTCGTGCAGGGAAAGACGAGCCGATCGCGATCGTGTCGATGGCGTGCCGGCTGCC
GGGAGGCGTTGCGACGCCGGACGACTACTGGCGTCTCTTGGCCGAGGGGAAGGACGCGAT
CGAAGGCCTCCCAGCGCGCTGGGACGGGTTCGAGGTCTACGACCCTGATCCGGAGGCCGC
AGGCAAGAGCTACGCGCGCGAAGGTGGGTTTGTTCGGGACATCGACCTGTTCGACGCGAA
CTTCTTCGGGATATCGCCGCGCGAGGCGCAGTCGATGGATCCGCAGCATCGGCTGGTGCT
GGAGACGGCGTGGGAGGCATTGGAGCGTGCCGGCGTGCGCCCGTCGGCGCTGAGCGGGAG
CGCCACCGGAGTGTACCTGGGTTCGATGGGCTCGGACTACGGTGCTCTTCATACTGTCGA
TCTGAAGGAGCTGGACGGGTACCGGGGCATCGGGAGCGCGGCGAGCATCCTCTCGGGCCG
GGTGGCCTACGCGCTGGGGCTGCAGGGCCCAGCGATGACGGTGGACACGGCGTGCTCGTC
GTCGCTGGTGTCGCTGCACCTGGCGTGCACGGCGCTGCGCCAGGGCGAATGCGACCTGGC
GCTGGCCGGCGGGGTGACGGTGATGAGCACCCCCGCGCTGTTCGTGGAGTTCAGCCGTCT
CAAGGGGATGTCCCGCGACGGTCGGTGCAAGAGCTTCTCGGTGCAGGCGGACGGCGCGGG
CTGGGCCGAGGGTTGCGGGATGCTGTTGTTGAAGCGGCTGTCTGACGCGCAGCGCGACGG
CGACCGTGTGCTGGGGGTGATCCGTGGCTCTGCGGTGAACCAGGACGGTCGCAGCCAGGG
TCTGACGGCGCCGAACGGCCCTGCCCAGCAGCGGGTGATCCGGCAGGCGCTGTCGTCGTG
TGGTCTGTCGCCCGAGGACATCGACGCGGTGGAGGCGCACGGTACGGGCACGAGCCTTGG
AGACCCGATCGAGGCCGGAGCGCTGGCGGAGGTGTTTGGACCGGAGCGTAGCCCCGAGCG
TCCGCTGTACCTGGGATCGTCGAAGTCGAACCTCGGACATGCGCAGGCGGCGGCGGGCGT
GGCGGGCGTGATCAAGATGGTGCTGTCGATGCAGCACGAGGTGCTGCCGAAGACGCTGCA
CGCGGAGCAGCCGAGCCCGCACATTGGGTGGGAAGGAAGCGGGCTGTCGCTGCTGCAAGA
GGCGCGTCCGTGGCGGCGCAACGGCCGGGTCCGTCGTGCCGGCGTGTCGTCGTTCGGGAT
CAGCGGGACGAACGCGCATATCATCCTCGAAGAAGCGCCGGCCGAGGCGCGGCGCGAGCC
TGTCGAGGCCGAGGCGGCGCCTGCGCTATTGCCGCTGGTGCTGTCGGGTCGAGACGAGGC
CGCGGTGAATGCGCAGGCGGGGCGGTGGGCGAAGTGGCTGGAAGAGCACGGGGAGGTGGG
GTGGTCGGACGTGGTGCGCACGGCGGCGCTGCACCGGACGCACTTCGAGTCGCGGGCGTC
GGTGCTTGCGGCGAGCGCTGCGGGAGCTGTGGAGGGTCTTCGCGCGCTGTCGTCGGGGCG
GCCGGATGCGGCGGTGGTGAGCGGGACGGCGAAGCGAGGCGGGAAGCTTGCGGTGCTGTT
CACGGGGCAGGGCAGCCAGCGGCTCGGGATGGGGAAGAGGCTTTACGAAGTGTACCCCGT
GTTCCGTGCGGCGTTCGACGAGGTGTGCGAGGCGCTGGACGCGTATCTCGACCGTGGGTT
GAGAGAGGTGGTGTTCGCGGCCGCGGGCAGCGAGGAAGGAGCGTTGCTGGAGCGGACGGA
GTACACGCAGCCCGGGCTGTTTGCGCTGGAAGTGGCGCTGTACCGTCAGTGGGAGTCGTG
GGGGCTGAAGCCCGCTGCGCTGCTGGGGCACTCGATAGGAGAGCTGAGCGCTGCGCATGT
GGCGGGTGTGCTGAGCCTTGCGGACGCAGCGAAGCTAGTGTGCGCCCGAGGTCGGCTGAT
GCAAGGGTGCGAGGCCGGGGGAGCGATGGTGTCGGTGGAGGCCTCGGAGCCGGAGGTGCA
GCGGGCGCTGTCGGAGGTCGGGGCGCAGGGGCGACTGAGCATCGCCGGGCTGAACGCGCC
GATGCAGACGGTGCTGAGCGGGGACGAAGCGGCGGTGCTCGCGGTGGCGCGGCGGCTGGA
GGCGCAGGGCCGGCGCACGCGGCGTCTGCGTGTGTCGCACGCGTTCCACAGCGCGCACAT
GGACGGGATGCTGGAGGAGTTCGGGAAGGTGGCGCGGGAGTGCACGTACGCGCGGCCACG
GCTGGCGGTGGTGAGCGGCGTGACGGGCGAGCTCGGTGGCGAAGAAGCGCTGATGTCGGC
CGAGTACTGGGTGAGGCAGGTGCGCGAGGCGGTGCGCTTCCTGGACGGGATGCGCACGCT
TGCGGCGGCGGGGGTGAGCACATACGTCGAGTGTGGGCCGGATGGCGTGCTGTGCGCGCT
GGGGGCGGGGTGCCTGCCGGAGGGAGCCGAGGCGACGTTTGTGACGAGCCTGCGGCGAGA
GCAGGAGGAAGAGCGCGCGCTGGCGACGGCGGTGGCGACAGTGCACGTGCAGGGGCACGA
GGTGGACTGGGCCCGGGTGCTGTCGGGCCGTGGCGGCCGGCCCGTGGAGCTGCCGACGTA
CGCGTTCCAGCGGCAGCGCTACTGGCTGGAAGCGCCGAAGAGCGCGGCGACCGAGGTGAA
TGTCTCCAGCGCGGAGCAGGCGCTGTGGAATGCGGCGCTGGAGGGCGAGGGAGATGGCGT
TGCGGAGCTGCTGGAGCTGCCTGACGACGTGCGCGCCAGCGTCGGGCCGCTGCTGCCGTA
CCTCGCGGCGTGGCGCCAGAGAAAGCAGGCAGAAGCGGCGGCGGCGAGCTGGCTGTATGA
GGAAGCGTGGCAAAACCGTCCGAGGCGTGTGACGGGTAGTCCGGATGTAAGGGGCACCTG
GCTCGTGGTGTCACCTCCGCTTGCCGGAGAGCTTGCGGAGGTGGTGCGTGGTGCGCTCGG
CGCCGCGGGGGCCGAGGTGATCGTGCACATCGCGGCCGTGGAGCGAGCGCAGCTCGCAGC
GTGGCTGAGAGAGCAAGCGCGCCTGAGAAAGGAGGAGGGCGAGCTGCGTGGCGTCATCGC
GCTCACGGCCTCAGGCGAGGAAGGCGCGCTGGAGCAAGGGCAGGCGCCCCGCAGCCTGTA
CCAGACGCTGGCGGTGGTGCAGGCGCTCGGCGACGCCGGAATCGGCGCGCGGCTGTGGTT
GCTCACGCAGGGAGCGGTCAGCACCGAGCCAAGCGAAGCGGTGGTGCACCCGTTGCAGGC
GCTGACGTGGGGACTGGGACGGGCGTTGGGGCTGGAGCACCCCGAGCGCTGGGGCGGGCT
GCTGGACGTGCCGGCGGAGCTGGACGCGGGCGTGATGCAGCACGTCTTGACCACGCTTGT
GTCCGACGACAACGAAGATCAGGTGGCGGTGCGGCGCGCCGGGCGCCTCGTGAGGCGCAT
TGTGCGTGTGCGCGGCGAGGGCGACGGCGAGGGCTGGAAGCCGCGCGGCACGGTGCTCAT
CACGGGCGGCGTGGGCGGGCTCGGAGGTCATCTTGCCCGCTGGCTGGCCGGGCGTGGCGC
AGAGCACCTTGTGCTGGCGTCGCGCCGCGGCGCGTCGGCGCCCGGGGCGAGCGAGCTGCG
GGACGAGCTTGTGGCGCGGGGCATTCGGGTGACGCTGGCGGCGTGTGACGTGTCGGAGCG
CGCGCAGCTCGCGGCGCTGCTCGCGGAGCTGGAGCAGGATGAAGCGCCGCTGAGGGCGGT
GGCGCACCTCGCGGGCATAGGCCGCCGTGTTCCGCTGCGAGAGCTCGAGCCTGAGCAGCT
CGAGCAGGAGCTCGCGGCGAAGGTGAAGGGGGCGTGGCACCTGCATCAGCTGCTGGGGAA
GCGGGAGCTGGATGCGTTCGTGCTCTATGGCAGCATCGCCGGGCTGTGGGGCTCCGGGGC
GCAGGCTGGGTACGGCGCAGCGAACGCAGGGCTGGACGCGCTCGCGCGGTACCGGCGCGC
GCGAGGGCAGGCTGCGACGGTGTTGCACTGCGGCCCCTGGGCGGGAGAAGGGATGGTGAC
CAGCGAGCTCCAGTCGCAGCTGCGGATCCGCGGGGTCGCGGTGATGTCGCCCGACAAGGC
GCTCGCCGGGCTGGAGATGGCGCTGCGGCTGGGGCGCACGTCGGTGGCGATCGCCGACGT
GGACTGGTCGCGCTTCGCGCCGTCGTTCAGCGCGGCGAGGCCGAGGCCGCTCCTGGACGG
GATCGAGGAGGCCCGGCGGGCGCAGGAGAGCCGCGGCCCGCAGCCGGCCGCAGGCGGGAC
CGCGCTGAGAGACACCTTGCTGGGCCTGAGCGAGGCCGAGCGGCGCGAGCGGGTACGTCA
GCTGGTGGCGAGCGAGACGGCCGCGGTACTGGGCATGACGGACCCGAGCCGGCTTGACCC
GGACCGTGGCTTTCTGGACCTCGGGCTGGATTCGCTGATGGCGGTGGAGCTGTCGAAGCG
GCTGCAGAAGCGCACGGGCATGACGGTACCGAGCACGCTGAGCTTCGATCACCCGACGCA
GAGCGACGTGGCGCGCTGGCTGCTGGAGCAGCTCACACCTCAGCCGCGACCGGAGCCGGC
GGTGCGCGAGGTGAGCCGGGAAGAGGGGTGGAGCACGCCGATAGCGATCGTGGGCGTGGG
GCTGCGCATGCCTGGCGGAGCGAGCGACCTGGAGAGCTTCTGGCAGGTGCTGGTCGAAGA
GCGGGATACGCTGCGGCCGATCCCGGCCCAACGATTCGACGTCGAGGCGCTGTACGATCC
TGACCCCGACGCGAAGGGCAAGACGTACGTGCGGAACGCGTCGCTGCTCGACGACGTGGC
GTCGTTCGACCCTGGGTTCTTCGGGATAGGTCCGCGGGAGGCGGAGCCGATGGATCCGCA
GCACCGGCTGCTGCTGGAGACGGCGTGGAGCGCCCTGGAGGACGCGGGGGTGCGTCCAGA
GCACCTGAAGGGCTCGGACACGGGAGTGTTCGTGGGCGTGGCGCCGAGCGAATACGCGAG
CTACCGAGGAAAGAGCGCGAACGAAGATGCGTATGCGCTGACAGGGACGGCGCTGAGCTT
TGCTGCGGGACGGGTGGCCTACCACCTCGGGCTGCAAGGCCCTGCGGTGTCGACCGACAC
GGCCTGCAGCTCGTCGCTGGTAGCGGTGCACCTGGCGTGCGACGCGCTGCGCCGGGGCGA
TTGCGAGGTGGCGCTGGCGGCAGGTGTGCAGGTGCTGGCGAACCCGGCGGGGTTTGTGCT
GCTGTCGCGCACGCGCGCGTTGTCGCCGGACGGGCGGTGCAAGGCGTTCTCGCAGGCGGC
GGACGGTTATGGCCGTGCCGAGGGAGTCGGGGTGCTGGTGCTGATGCGGCTGTCCGAGGC
GCAGCAGCAGGGGAAGCGGGTGCTGGGTGTGGTGCGCGGCACGCCGGTCAATCAGGACGG
CGCGAGCAGCGGGATCACGGCGCCGAACGGCACGGCGCAGCAGAAGGTGGTGCGCGCGGC
GCTGCGGAACGCGGGGCTGGAGCCGGCGAGCATCGATGTGGTGGAGTGCCACGGTACGGG
CACGTCGCTGGGCGACCCGATCGAGGTGCAGGCGCTCGGCGCGGTGTACGGGCAAGGTCG
GGATATGGCTCGTCCGCTGCAGCTGGGCGCGGTCAAGAGCAATATCGGTCATCTCGAGTC
CGCCGCGGGCATCGCAGGGGTGTGCAAGATCCTGGCGGCGTTCCGTTACGAGTCGCTGCC
GGCGACGCTGCACAGCTCGCCGCGCAATCCCCGCATCCCGTGGGAGAACCTGCCGGTGCA
GGTGGTCGATCGCCTGACCCCCTGGCCTCGGCGCGCAGAGGGCCCCCCGCGCCCTGCCGG
CGTGTCGTCGTTCGGGATCAGCGGGACGAACGCGCATGTCATCCTCGAAGAAGCGCCGGC
CGAGGCGCGGCGCGAGCCTGTCGAGGCCGAGGCGGCGCCTGCGCTATTGCCGCTGGTGCT
GTCGGGTCGAGACGAGGCCGCGGTGAATGCGCAGGCGGGGCGGTGGGCGAAGTGGCTGGA
AGAGCACGGGGAGGTGGGGTGGTCGGACGTGGTGCGCACGGCGGCGCTGCACCGGACGCA
CTTCGAGTCGCGGGCGTCGGTGCTTGCGGCGAGCGCTGCGGGAGCTGTGGAGGGTCTTCG
CGCGCTGTCGTCGGGGCGGCCGGATGCGGCGGTGGTGAGCGGGACGGCGAAGCGAGGCGG
GAAGCTTGCGGTGCTGTTCACGGGGCAGGGCAGCCAGCGGCTCGGGATGGGGAAGAGGCT
TTACGAAGTGTACCCCGTGTTCCGTGCGGCGTTCGACGAGGTGTGCGAGGCGCTGGACGC
GCATCTCGACCGTGGGTTGAGAGAGGTGGTGTTCGCGGCCGCGGGCAGCGAGGAAGGAGC
GCAGCTGGAGCGGACGGAGTACACGCAGCCCGGGCTGTTTGCGCTGGAAGTGGCGCTGTA
CCGTCAGTGGGAGTCGTGGGGGCTGAAGCCCGCTGCGCTGCTGGGGCACTCGATAGGAGA
GCTGAGCGCTGCGCACGTGGCGGGTGTGCTGAGCCTTGCGGACGCAGCGAAGCTAGTGTG
CGCCCGCGGTCGGCTGATGCAGGGGTGCGAGGCCGGGGGAGCGATGGTGTCGGTGGAGGC
CTCGGAGCCGGAGGTGCAGCGGGCGCTGTCGGAGGTCGGGGCGCAGGGGCGACTGAGCAT
CGCCGGGCTGAACGCGCCGATGCAGACGGTGCTGAGCGGGGACGAAGCGGCGGTGCTCGC
GGTGGCGCGACGGCTGGAGGCGCAGGGCCGGCGCACGCGGCGTCTGCGTGTGTCGCACGC
GTTCCACAGCGCGCACATGGACGGCATGCTGGAGCAGTTCGCGAAGGTGGCGCGGGAGTG
CACGTACGCGCGGCCGCAGCTGGCGGTGGTGAGCGGCGTGACGGGCGAGCTCGGTGGCGA
AGAAGCGCTGATGTCGGCCGAGTACTGGGTGAGGCAGGTGCGCGAGGCGGTGCGCTTCCT
GGACGGGATGCGCACGCTTGCGGCGGCGGGGGTGAGCACATACGTCGAGTGTGGGCCGGA
TGCCGTGCTGTGCGCGCTGGGGGCGGGGTGCCTGCCGGAGGGAGCCGAGGCGACGTTTGT
GGCGAGCCTGCGGCGAGAGCAGGAGGAAGAGCGCGCGCTGGCGACGGCGGTGGCGACAGT
GCACGTGCAGGGGCACGAGGTGGACTGGGCCCAGGTGCTGTCGGGCCGTGGCGGCCGGCC
CGTGGAGCTGCCGACGTACGCGTTCCAGCGGCAGCGCTACTGGCTGGAAGCGCCGAAGGC
GCGTACCGACGTGGGCTCGGCGGGCTTGAGGGAGTCGGGGCATCCGCTGCTCGGAGCGGC
AACGAAGCTGGCCGACGGCGACGGCCATCTATTCACAGGCCGGCTGTCGCTGGGCGAGCA
GCCGTGGCTTCGCGACCATGCGGTGTTTGGCGAGGTGGTCTTCCCGGGCACGGGGATGCT
GGACCTCGCGCTGGCGGCTGGGCGCACGGTGGGCAGCGGGGCGCTGTCGGAGCTCACGAT
CTCCGAGCCGCTGATGCTCGCGGAGGACGTGGCCGTGCGGCTGCAGCTCTCGGTCGGGGC
GCCCGACGCCGCGGGGCGGCGTGCGTTTGGGCTGTACAGCCAGCCGGAGCAGGGACCGGG
AGATGCCCCCTGGGTGCAGCACGCGACGGGCGTGTTGACCGACGAGACCCTCGCCACCTC
CGGCGAGCTCGATGAGCTGACGACGTGGCCAGTGCCCGGCGCCGAGGCGGTGGACCTCTC
CGGGTTCTACGAGCGGCTGCATGAGCGTGGACTCCGCTACGGCCCGGCCTTCCAGGGGCT
CGTGGAGCTGTCGCGTCGAGACGCGACCTTCTTCGGCCGGGTGGTGTTGCCCAAAGACGC
GACCGACAGCGCCGAGGACTACGGGGTGCATCCGGCGCTGATGGACGCCGCGCTGCATAC
GATGGTCGCAGCGTTTGCGGAGGTATCAGCGCCGGACGACGTGCTGCTGCCTTTCTCGTG
GTCGGACGTGGCGTTGCACGCCACGGGGGCGAGCGAGCTCCGGGTGAGGCTGGAGCTCGC
AGGAGGCAGAGACTCGGCACAGGCAGCCGCCTCGCTGCGCGTTACAGATGCCGCCGGCCA
GCCGGTGGTGAGCGTCGGTGCCCTGCATCTGCGCCGGGCGACGGCCGAGCAGCTGCGGGC
AGCGACGCATGCCGAGGCGCAGCACCTGTACCGGGTGGACTTCCAGCTCGTGAGCCTCGT
GGAGGCGGGCTCGAAGGTGGACTCGCTGGTGGTGCTCCGTGCGCCTGAGGGGCGAGGGCG
ACTGGGCGAAGCGCTGGGTGTGGAGGCGATCGCAGGCCTCGATGCATTGCTCGCGCGGAT
CGAGCAGGGAACCCGATTGCCTGAGCGGGTGCTGGTCGACATGACGGCTGGCAGCTCACA
GCGCTCGGACATGGTGATATCGTCGCACGAGGCGACGGGACAGGCGCTGTCGCTGCTGCA
AGCGTGGCTGTCGGAGCCCCGGCTCGAGGGGGTGGAGCTGGTGTGGGTGACGCGAGATGC
GGTCAGCGCCGCTCCGGGCGACGGTGTCCAAGACCTGGCGCACGCGCCGCTGTGGGGGCT
TGTTCGCACGGCGCGAAGCGAGCACCCCGAGCGCCGGCTGCGCCTGATCGACGTTGGGAC
CGAGCCTCTGCACGGCGGGCTGCTGGCGCGCGCGCTGGCGACGGCGACGGAGCCGGAGCT
TGCGCTGCGTGGCGGCGCGGCGATGGCGGCGCGCCTGGTGCGCGTGCCGGCGGCAGCGGA
AGGGCTCACGCCGGCGCGCGGGCTGGACCCGACGGGCACGGTCCTGGTGACCGGAGGAAC
AGGCGAGCTGGGTCAGGCCGTCGCGGAGCATCTGGTGCGCGCACACGGGGTGCGGCACCT
CGTGCTGACGTCGCGCCGTGGGCTGGAGGCGCCCGGGGCCCCAGGCTTCGTGCAGGCGCT
GGAGAAGCTCGGTGCCGAGACCGTGACGGTGGCGGCGTGTGACGTGTCGAAGCGGGAGGA
GGTCGCGCGCGTGCTGGCCGGCATCGAGGCCGCACATCCGCTGACCGCGGTGCTGCACCT
GGCCGGCGTGCTCGACGATGGCGTCATCACCGCGCAGACGCCCGAGCGTCTCTCGCGGGT
GCTGGCGCCGAAGGTGAACGGGGCGCTGCACCTGCACGAGCTGACAGAGGATCTCGATCT
CTCGGCCTTCGTGCTGTTCTCCTCGATGTCCGGGACGCTCGGGACGGCGGGCCAGAGCAA
CTACGCGGCGGCCAACAGCTTCCTCGACGCGTTCGCGGCGCATCGCCGCAGCCGCGGGCT
CGCGGCGACGAGCCTGGCGTGGGGCTTCTGGGCGCAAACGGGCGTGGGCATGACAGCGCA
CCTGGGCGAGGCGGAGCTCTCACGTATCCAGCGCGCCGGACTTGTGCCGATACGGGTCGA
GGAGGGCCTTTCGCTGCTGGACGCCGCGCTTCTGCGCCCCGAAGCGAGCCTGGTGCCTGC
GCACCTCGATCTTGCGCAGATGCAGCGGGGGCTGGAGGCCAGCGGCGAGCTGCCCGCGCT
GCTTCGCGCGCTGCTGCGCCCTGGGCTGCGCAAGGCGTCATCCGCCACGAGGAAGGAAGC
CTCGGCGCTCCGCGAGCGCCTCTCGGAGCTGCCGGAGGCGGAGCGCCTGAGCTCGCTCGT
CGAGCTGGTTCGGGCCGAGGTGGCCGCGGTGCTCGGGCTGCCGCGCAGCGAGGCCGTTGC
GGTAGATCAGGTGCTGAAGGACCTAGGGCTAGATTCGTTGATGGCGGTGGAGCTGCGCAG
TCGGCTCAGCGCCCGAGCCGAGATCCCCCTCCCGGCGACGCTGGTGTTCGACTACCCGAC
GCCGCGCGCCGTCGCAGAGCTGCTCCTGAGACAGGCTTTCTCGAAGCAGCAGGTGACGGC
AGCGCGGGCGCGTCGCCGGACGAAGGAAGACGAGGCGATCGCGATCGTATCGATGGCGTG
CCGGTTGCCAGGGGGCGTGGCGACGCCGGAAGACTACTGGCGTCTCCTGGCGGAAGGGAA
GGACGCCATCGAGCGCTTTCCCTCCCGTTATGACGCGTTCTCTGTTTATGACCCCGATCC
GGAGGCGGTGGGCAAGAGCTACGTGCGCGAGGGTGGATTCCTGCGGGATATCGATGTCTT
CGACGCAGGCTTCTTCGGGATCTCGCCGCGCGAGGCGCAGGCGATGGATCCCCAGCAGCG
GCTGGTGCTGGAGACGGCGTGGGAGGCGCTGGAGCGAGCCGGCGTGCGGCCCTCGATGCT
GAGCGAGAGCGCCACCGGGGTATACCTGGGCTGGATGGGCTCGGACTACGGTGCTCTTCT
CGGCAATGACCTCGCCGCGCTGGACGGGTACCAGGGTACGGGGAGCGCGGCGAGCGTGCT
TTCAGGCCGGGTGGCTTACGTGCTGGGGCTTCAGGGCCCAGCGATCACGGTGGACACGGC
GTGCTCGTCGTCGCTGGTGTCGCTGCACCTGGCGTGCACGGCGCTGCGCCAGGGCGAATG
CGACCTGGCGCTGACCGGCGGGGTGATGGTGATGACCACGCCCGCGGGATTCGTTGAGTT
CAGTCGTGCCCGGGGGCTTGCGCGAGACGGTCGGTGCAAGAGCTTCTCTGCCCAGGCTGA
CGGCGTCATCTGGTCCGAAGGGTGCGGGATGCTGTTGCTGAAGCGGCTGTCTGACGCGCG
GCGCGACGGCGACCGTGTGCTGGGGGTGATCCGTGGCTCTGCGGTGAACCAGGACGGTCG
CAGCCAGGGTCTGACGGCGCCGAACGGCCCTGCCCAGCAGCGGGTGATCCGGCAGGCGCT
GTCGTCGTGTGGTCTGTCGCCCGAGGACATCGACGCGGTGGAGGCGCATGGGACGGGTAC
GAGCCTCGGAGACCCGATCGAGGCCGGAGCGCTGGCGGAGGTGTTTGGACCGGAGCGTAG
CCCCGAGCGTCCGCTGTACCTGGGGTCGTCGAAGTCGAACCTGGGACATGCGCAGGCGGC
CGCGGGTGTGGCGGGCGTGATCAAGATGGTGCTGGCGCTGCAGCACGAGGTGCTGCCGAA
GACGCTGCATGCGGAGCAGCCGAGCCCGCACATCGCGTGGGAGGGGAGCGGGCTGTCATT
GCTGCAAGAGGCGCGTCCGTGGCGGCGCAACGGCCGGGTCCGTCGTGCCGGCGTGTCGTC
GTTCGGGATCAGCGGGACGAACGCGCATATCATCCTCGAAGAAGCGCCGGCCGAGGCGCG
GCGCGAGCCTGTCGAGGCCGAGGCGGCGCCTGCGCTATTGCCGCTGGTGCTGTCGGGTCG
AGACGAGGCCTCGGTGGCGGCGCAGGCGGGGCGGTGGGCGAAGTGGCTGGAAGAGCACGG
GGAGGTGGGGTGGTCGGACGTGGTGCGCACGGCGGCGCTGCACCGGACGCACTTCGAGTC
GCGGGCGTCGATGCTTGCGGCGAGCGTGTCCGAGGTGGTGGAGGTGCTGCGGGCGCTGTC
AGAGGGTCGCGGCCACCGGGCGGTGTCCGTGGGCACGGCGCGTGCGCGAGGCAAGGTGGT
GTTCGTGTTCCCCGGCCAAGGGAGCCAGTGGCCGGGGATGGGCCGGGCGCTGCTGGAGCA
GAGCGCAGCGTTCGCGGAGGCGGTGCAGGCGTGCGATGAGGCGCTGCGGCCGTCCACGGG
CTGGTCTGTGCTGTCGGTGCTGCGCGGAGATGGCGGGGAGGAGCAGCCGTCGCTGGAGCG
GGTGGACGTGGTGCAGCCTGCGCTGTTCGCGATGTGCGTGGGTCTGGCCGCGGCGTGGCG
GTCGCTGGGGCTGGAGCCTGCGGCGGTGGTGGGCCACAGCCAGGGCGAGGTGTCCGCGGC
GGTGGTGTGCGGGGCGCTGTCGCTTGCGGAGGGAGCGCGGGTAGTGGCGCTGCGCAGCCA
GGCGGTGCGGCAGCAGTCGGGGATGGGGGCGATGATGCTGGTCGAGCAGCCGGTGTCGGA
GGTGCAGGAGCGCATCGCGCCGTACGGGGAGGCGCTTGCGATAGCGGCGGTGAACACGTC
GAACTCGACGGTGGTGTCGGGTGACGTGGAGGCGGTGGACGGGCTGATGGTGGAGCTGAC
GGCAGAAGGTGTGTTCTGCCGGAAGGTGAACGTCGACTACGCGTCGCACAGCGCGCACAT
GGATGCGCTGCTGCCCGAGCTAGGAGCGAAGCTGTCGTCGCTCAGGCCGAAGGCGACGCA
GCTGCCGTTTTACTCGACGGTGACAGGAGAGGTGTCGCGGGGCGAGGCGCTCGACGGCGA
GTACTGGTGCCGCAACCTTCGGCAGACGGTGCGCCTGGACCGAGCGCTGTCGAAGCTGCT
GGAGGACGGGCACGGTGTGTTCGTGGAGGTGAGCGCGCACCCGGTGCTGGCGATGCCGCT
GACGACGGCGTGCGGGGAGGCGCAGGGGGTGGTGGTGGGGAGCTTGCAGCGCGACGAAGG
TGGGTTGTCGCAGCTGTACAGGACGCTCGGGCAGCTGCACGTGCAGGGGCACGAGGTGGA
CTGGGCACGGGTGCTGTCGGGCCATGGTGGTCGTGCCGTGGAGCTGCCGACGTACGCGTT
CCAGCGGCAGCGCTACTGGCTGGATATCTCGAAGGCGCGTAGCGACGTGAGCTCGGCGGG
GCTGAAGGCGGCCGCCCATCCGCTGCTGGGAGCAGCAACGAAGCTGGCTGAGGGCGATGG
CCATCTGTTCACCGGCCGGCTGTCGCTGGGCGAGCATGCGTGGCTCCGCGACCATGAGGT
GTTTGGTAACGTGGTGTTCCCCGGCGCGGGGATGCTGGAGCTTGCGCTGGCGGCTGGGCG
CACGGTGGGCAGCGGGGCGCTGTCGGAGATGGTCTTGGCCGAGCCGCTGGTGCTCGCCGA
GGACGTGGCCGTGCGACTGCAGCTGTCGGTCGGCGCGCCGGACGCGGCGGGCCGGCGTGA
GTTTGGGCTGTACAGCCAGCGGGAGCAGGGGCCAGAAGACGCCCCGTGGGTGCAGCACGC
GACGGGGGTGTTGACGGACGAGCCCCGCGGCATCCCTGGTGACCTCGATGAGCTCGCGAC
GTGGCCAGTGCCCGGCACGGAGGCGGTGGAGCTCTCCGGGTTCTACGAGCGGCTGCGTGA
GCGTGGACTCCACTACGGCCCGGCCTTCCAGGGGCTCGTGGAGCTGTGGCGTCGAGGCGC
GGCGTACTACGGCCGGGTGGCGTTGCCCAAGGCCGCGGGGGACAACGCCGAGGACTACGG
CGTGCATCCGGCGCTGATGGACGCCGCGCTGCACACGATGGTCGCAGCCTTCTCGGAGAT
GGCAGAGCAAGGCGCCGTCTTGTTGCCGTTCTCCTGGTCGGATGTGGTGTTGTACGCCGT
GGGCGCGAGCGAGCTCCGTGTCCGGATGGAGCTCCGGGAACAAGGCGCACAGCAGGAGGT
CACTTCGCTGCACGTCGCTGACCCGACCGGCCAGCTGGTGGCGAGCGTCGGCGCCTTGCA
TCTGCGCCGGGCGACGGCCGAGCAGATGCGGACAGCCATCCATGCTGGTGTCCAGCATCT
GTACCGGGTGGACTTCCAGCCTGCGGAGCTCACGGCGTCGGTCGCGGAGACGGGCTCGCT
GGCGGTGCTCGGTGCGCCGGAGGGGGGAGGACGACTGGCCGAGGCTCTGGGGGCCGAGGT
GGTTGCAGGCCTCCACTCGCTGGTCGCACTGATCGAGCAGGGTGCCCGACGGCCTGTGCG
GGTGCTGGTCGATGCCACTGCTGCAAATGCCGATCGATCGCCAGTAGCGGCGTCGCACGA
GGCGGCGCGAGAGGCGCTGTCGCTGCTGCAAGCGTGGCTGTCGGAGCCCCGGCTCGAGGG
GGTGGAGCTGGTGTGGGTGACGCGGGATGCGGTCAGCGCTGGTCCGGGCGACGGTGTCCA
GGACCTGGCACACGCACCGCTGTGGGGGCTTGTTCGCACGGCGCGAAGCGAGCACCCAGA
ACGCGGCCTGCGCTTGATCGATGTCGGGACCGAGCCTGTGGACGGCGAGCTGCTGGCGCG
CGCGCTGGCGACGGCGACGGCGCCGGAGCTTGCGCTGCGCGGCGGCGCTGCGCTGGCCGC
GCGCCTGGTGCGCGTGCCGGCGGCAGCGGAGACGCTTACGCCGGCTCGCGGGCTGGACCC
GGCAGGCACGGTCCTGGTGACCGGGGGAACAGGCGAGCTGGGTCAGGCCGTTGCGGAGCA
TCTGGTGCGCGCGCACGGAGTGCGGCACCTTGTGCTGACGTCGCGCCGTGGGCTGGAGGC
GCCCGGGGCCCGGGAGCTCGTGCAATCGCTGGAGAAGCTCGGCGCCGAGACCGTGACGGT
GGCGGCGTGTGACGTGTCGAAGCGAGAGGAGGTCGCGCAGGTGCTGGCCGGAATCGAGGC
CGCGCACCCGCTGACCGCGGTGCTGCACCTAGCCGGCGTGCTCGACGACGGCGTCCTGTC
GTCGCAGACGCCGGAGCGCATTTCACGGGTGTTCGCACCGAAGGTGGACGGGGCGCTGCA
CCTGCACGAGCTGACGCGGGAGCTCGACCTCTCGGCGTTCGTGCTGTTCTCGTCGGCGGC
CGGGACGCTCGGGACGTCGGGCCAGAGCAACTACGCGGCGGCCAACAGCTTCCTTGACGC
GCTCGCGGCGCACCGCCGCAGCCGCGGGCTCGCGGCGACGAGCCTGGCGTGGGGCTTCTG
GGCGCAAGCGGGCGTGGGCATGACAGCGCACCTGGGCGAGGCAGAGCTGTCGCGCATGCG
GCGCAGCGGGGTTGTGCCGATGTCCGTGAGAGAAGGGCTGTCGCTGCTGGACACCGCGCT
CCTGCGCTCCGAGGCGACCCTGGTGCCTTTGCACCTCGATGTTGCGCAGCTACAGCGGGG
GCTGGAGGCCAGCGGCGAGCTGCCGGCGTTGTTCAGGTCCCTGCTGCGCCCGGGCCTGCG
CAAGGCGTCCTCCGCGACGAAGAAAGAGGCGTCTGCGCTCCGCGAGCGCCTCTCGGAGCT
GCCGGAGGCGGAGCGCCTGAGCTCGCTCGTCGAGCTGGTTCGGGCCGAGGTCGCCGCGGT
AATCGGGCTGCCGCGCAGCGAGGGTGTTGAGGCAGACCAGGTGCTGAAGGACCTGGGGCT
CGACTCGCTGATGGCGGTGGCGCTGCGCAACCGCCTCACGTCCCGTACCGACACGTCCTT
GCCGGCGACGCTGGTCTTTGACTACCCGACGCCGCGGGCCATCGCGAGGCTGCTGGACAG
CCGCCTGCTCTCGGCCCTCTCCCCCCAGGAGGACGGCCCTCCCGCGGACCCGGACGGCAT
GTTGAAATGGGTGCTCAAGCGGGTCTCCGCGAGCCAGATGCAGCAAGCTGGTGTGCTCCA
GCGGCTCCTCCAGCTCGCCGAGCCGAAGCTCCCCCGAACGAACGGTGGCCACCACGAGAA
TGGTAGCCGCCAAGAAGAAGAGGATGTCCCGCTCCCGCTGACCATGCAGGACATCGACAG
TAAGCTCGACGCAATTTTGCGTGGTGAGTGATGACAAAGCCCGATGATACCCGACTCCAG
CGCGCCATGGCGGCGATCGCTCTGCTCGAGCGGCGCATGGCAGAGCTCGAGGCCTCGCGC
ACCCAGCCCATCGCGATCGTATCGATGGCGTGCCGGCTGCCAGGGGGCGTGGCGACGCCG
GAAGACTACTGGCGTATCCTGGCGGAAGGGAAGGACGCGATCGATGGCTTCCCGTCCCGA
TGGGAGTCGTTCTCTGTTTACGACCCCGATCCGGACGTCGTGGGCAAGAGCTACGTGCGC
GAGGGTGGATTCCTGCGGGATATCGATGTCTTCGACGCGGGCTTCTTCGGGATCTCGCCT
CATGAGGCGCAGGGGATGGACCCCCAGCAGCGGCTGGTGCTGGAGACGGCGTGGGAGGCG
CTGGAGCGGGCCGGCGTGCGCCCCTCGGCGCTGAGCGAGAGCTCCACCGGAGTGTACCTG
GGCTCGATGGGCTCGGACTACGGTGCTCTTCTCGGCAATGACCTCGCCGCGCTGGACGGG
TACCGAGGGACCGGGAGCGCGGCGAGCGTGCTTTCAGGCCGGGTGGCTTACGTGCTGGGG
CTTCAGGGCCCAGCGATCACGGTCGACACGGCGTGCTCGTCGTCGCTGGTGTCGCTGCAC
CTGGCGTGCACGGCGCTGCGCCAGGGCGAATGCGACCTGGCGCTGGCCGGCGGGGTGACG
GTGATGAGCACCCCCGTGCTGTTCGTGGAGTTCAGCCGCCTCAAGGGGATGGCCCGAGAC
GGTCGGTGCAAGAGCTTCTCGGCGCGGGCGGACGGCGCGGGCTGGGCCGAGGGTTGTGGG
ATGCTGTTGCTGAAGCGGCTGTCTGACGCGCGGCGCGACGGCGACCGTGTGCTGGGGGTG
ATCCGTGGCTCTGCGGTGAACCAGGACGGTCGCAGCCAGGGTCTGACGGCGCCGAACGGC
CCTGCCCAGCAGCGGGTGATCCGGCAGGCGCTGTCGTCGTGCCGGCTGTCGCCCGAGGAC
ATCGACGCGGTGGAGGCGCATGGGACGGGCACGAGCCTCGGAGACCCGATCGAGGCCGGA
GCGCTGGCGGAGGTGTTCGGCCCGAAGCGTAGCCCGGAGCGTCCGCTGTACCTGGGGTCG
TCGAAGTCGAACCTGGGACATGCGCAGGCGGCCGCGGGTGTGGCGGGCGTGATCAAGATG
GTGCTGGCGCTGCAGCACGACGTGCTGCCGCGAACGCTGCATGCGGAGCAGCCGAGCCCG
CACATTGGGTGGGAAGGAAGCGGGCTGTCGCTGCTGCAAGAGGCGCGTCCGTGGCGGCGC
AACGGCCGGGTCCGTCGTGCCGGCGTGTCGTCGTTCGGGATCAGCGGGACGAACGCGCAT
ATCATCCTCGAAGAAGCGCCGGCCGAGGCGCGGCGCGAGCCTGTCGAGGCCGAGGCGGCG
CCTGCGCTATTGCCGCTGGTGCTGTCGGGTCGAGACGAGGCCTCGGTGGCGGCGCAGGCG
GAGCGCTGGGCGAAGTGGCTGGAAGAGCACGCOGAGGTGGGGTGGTCGGACGTGGTGCGC
ACAGCAGCGCTGCACCGGACGCACTTCGCCTCTCGCGCATCGGTGCAGGCGGCGAGCGTG
TCCGAGGCGGTGGAGGTGCTGCGGGCGCTGTCGCAGGGTCGCGGCCACCGGGCGGTGTCG
GCGGGTACGGCGCGTGCGCGAGGCAAGGTGGTGTTCGTGTTCCCCGGCCAAGGGAGCCAG
TGGCCGGGGATGGGCCGGGCGCTGCTGGAGCAGAGCGCAGCGTTCGCGGAGGCGGTGCAG
GCATGCGACGAGGCGCTGCGGCCGTGGACGGGCTGGTCTGTGCTGTCGGTGCTGCGCGGC
GAGGCGGGTGAGGCAGGTGAGGAGCAGCCGTCGCTGGAGCGGGTGGACGTGGTGCAGCCC
GCGCTGTTCGCGATGTGCGTGGGTCTGGCCGCGGCGTGGCGGTCGCTGGGGCTGGAGCCT
GCGGCGGTGGTGGGCCACAGCCAGGGCGAGGTGTCGGCGGCGGTGGTGTGCGGAGCGCTG
TCGCTTGCGGAGGGAGCGCGGGTAGTGGCGCTGCGCAGCCAGGCGGTGCGGCAGCAGTCG
GGGATGGGGGCGATGATGCTGGTCGAGCGGCCGGTGTCGGAGGTGCAGGAGCACATCGCG
CCGTACGGGGAGGCGCTTGCGATAGCGGCGGTGAACACGTCGAGCTCGACGGTGGTGTCG
GGTGACGTGGAGGCGGTGGACGGGCTGATGGTGGAGCTGACGGCAGAAGGTGTGTTCTGC
CGGAAGGTGAACGTCGACTACGCGTCGCACAGCGCGCACATGGATGCGCTGCTGCCCGAG
CTAGGAGCGAAGCTGTCGTCGCTCAGGCCGAAGGCGACGCAGCTGCCGTTTTACTCGACG
GTGACAGGAGAGGTGTCGCGGGGCGAGGCGCTGGACGGCGAGTACTGGTGCCGCAACCTT
CGGCAGACGGTGCGCCTGGACCGAGCGCTGTCGAAGCTGCTGGAGGACGGGCACGGTGTG
TTCGTGGAGGTGAGCGCGCACCCGGTGCTGGCGATGCCGCTGACGACGGCGTGCGGGGAG
GCGCAGGGGGTGGTGGTGGGGAGCCTGCAGCGCGACGAAGGTGGGTTGTCGCAGCTGTAC
AGGACGCTGGGGCAGCTGCACGTGCAGGGCCACGAGGTGGACTGGGCACGGGTGCTGTCG
GGCCACGGCGGTGGTGTCGTGGAGCTGCCGACGTACGCGTTCCAGCGGCAGCGCTACTGG
CTGGATATCTCGAAGGCGCGTAGCGACGTGAGCTCGGCGGGGCTGAAGGCGGCCGCCCAT
CCGCTGCTGGGAGCGGCAACGAAGCTGGCTGAGGGCGATGGCCATCTGTTCACCGGCCGG
CTGTCGCTGGGCGAGCATGCGTGGCTCCGCGACCATGAGGTGTTTGGTAACGTGGTGTTC
CCCGGCGCGGGGATGCTGGAGCTTGCGCTGGCGGCTGGGCGCACGGTGGGCAGCGGGGCG
CTGTCGGAGATGGTCTTGGCCGAGCCGCTGGTGCTCGCCGAGGACGTGGCCGTGCGACTG
CAGCTGTCGGTCGGCGCGCCGGACGCGGCGGGCCGGCGTGAGTTTGGGCTGTACAGCCAG
CTGGAGCAGGGGCCAGAAGACGCCCCGTGGGTGCAGCACGCGACGGGCGTGTTGGCGGAC
GAGCCCCGCGGCATCCCTGGTGAGCTCGATCAGCTCGCGACGTGGCCAGTGCCCGGCGCG
GAGGAGGTGGATCTCTCCGGGTTCTACGAGCGGCTGCGTGAGCGTGGACTCCACTACGGC
CCGGCCTTCCAGGGGCTCGTGGAGCTGTGGCGTCGAGGCACGACGTTGTTCGGCCGCGTG
GTGTTGCCCAAGGCCGCGGGGGACAGCGCCGAGGACTACGGCGTGCATCCGGCGCTGATG
GACGCCGCGCTGCATACGATGGTTGCGGCCCTCTCCGAGAGGCCAGGAGCGAACGCGGTG
CTCCTGCCGTTCGCTTGGTCGGACGTGGTGTTGCTCGCCATGGGGGCGAGCGAGCTCCGG
GTCCATGTGGATCTCCAGGATCACGGCGCAGAGCAGGCGATGGCTTCGCTGTACGTCGCT
GACTCCGTGGGGCAACTTGTGGTGAGCATCGGCGAGCTGAAGCTGCGCTGGGCGACGGCC
GAGCAGCTGAGGGAGGCGACCCGTGCCGAGGCGCAGCATCTGTACCGGGTGAACTTCCGC
CCTGTGCGTCTCGTGGACGGTTCGTCGGAGTCGGCCCCGCCAATGCTTGTCATAGTCTCG
GAGGGGCAAGGGCAACTGGCCGAGATCCTGGAGGCGGAAGCGGTCGCGAGCCTCGATGTA
CTGCTTGCACGTCTCGCGCAGGGCGCGAGCGCGCCTGTGCGGGTGCTGGTCGATGCCACT
GCTGCAAATGCCGGTCGATCGCCAGTAGCGGCGTCGCACGAGGCGGCGCAAGAGGCGCTG
TCGCTGCTGCAAGCGTGGCTGTCGGAGCCCCGGCTCGAGGGGGTGGAGCTGGCGTGGGTG
ACGCGAGATGCGGTCAGCGCCGCTCCGGGCGACGGTGTCCAGGACCTGGCACACGCGCCG
CTGTGGGGGCTTGTTCGCACGGCGCGAAGCGAGCACCCCGAGCGCCAACTGCGCCTGATC
GACGTCGGGACCGAGCCTGTGGATGGCGGGCTGCTGGAGCGCGCGCTGGCGACGGCGACG
GAGCCGGAGCTTGCGCTGCGCGGCGGCGCTGCGCTGGCCTCGCGCCTGGTACGCGTGCAG
GCGGTGGAGGAGGTCACCCGAACTCGCGGGCTGGACCCGGCAGGCACGGTCCTGGTGACC
GGGGGAACAGGCGAGCTGGGTCAGGCCGTTGCGGAGCATCTGGTGCGCGCGCACGGAGTG
CGGCACCTTGTGCTGACGTCGCGCCGTGGGCTGGAGGCGCCCGGGGCCCGGGAGCTCGTG
CAATCGCTGGAGAAGCTCGGCGCCGAGACCGTGACCGTGGCGGCGTGTGACGTGTCGAAG
CGGGAGGAGGTCGCGCAGGTGCTGGCCGGAATCGAGGCTGCGCACCCGCTGACCGCGGTG
CTGCACCTGGCCGGCGTGCTCGACGACGGCGTCCTGTCGTCGCAGACGCCGGAGCGCATT
TCACGGGTGTTCGCGCCGAAGGTGGACGGGGCGCTGCACCTGCACGAGCTGACGCGGGAG
CTCGACCTCTCGGCGTTCGTGCTGTTCTCGTCGGCGGCCGGGACGCTCGGGACGTCGGGC
CAGAGCAACTACGCGGCGGCCAACAGCTTCCTCGACGCGCTCGCGGCGCACCGCCGCAGC
CGCGGGCTCGCGGCGACGAGCCTGGCGTGGGGGTCGTGGATGCCGGAGCCGACGGGGACC
GCCCGACCGAGCACCGCCGAGCCGGGAGCCACGAAACAACCTGGTCTGATGCCGATGTCC
TTCAGCGCGGGGTTGTCGCTTCTGGACGCGACGCTCTCGCGGCCTGAAGCGAACCTGGTT
CCGGCGTACCTGGATCTCGCCAGGCTGCAGAGAGGAGTCGAGGCCAGCGGCGAGCTGCCT
GCGCTGCTCCGGGCGCTGCTGCGTCCGGGCCTGCGCAAGGCTGCGTTTGGAGCCCAGGAA
GCCTCGGCGCTCCGTGAGCGTCTCGCGAGGCTGCCGGAGTCGGAGCGCCTGAATGCGCTC
GTCGCGCTGGTTCAGGCCGAGGTCGCGACCGTGATGGGGCTGCCGCGCAGCGAGGCTGTG
GCAGCAGATCAGGTGCTCAAGGAGCTGGGGCTCGACTCGCTGATGGCGGTGGCGCTGCGC
AACCGCCTCACCGCCCGGACCGAGACGCCATTGCCGGCGACGCTGGTCTTTGACTACCCG
ACGCCGCGGGCCGTCGCGGAGCTGCTGCTGAAGCAGGCGTTCTCGGAGCTGAAGAGCGCG
GGGGCCCGCCCGCGGGGGCGTCGTCGCGGGCAGGAGGACGAGCCCATCGCGATCGTGTCG
ATGGCGTGCCGGCTGCCAGGGGGCGTGGCGACGCCGGAAGACTACTGGCGTCTCCTCGCG
GAAGGGAAGGACGCGATCGAGCGCTTCCCTGCACGCTGGGACGCCCTAAGTATCTACGCT
CCTGATCCGGACGCCGTGGGCAAGAGCTATGCGCGCGAGGGGGGCTTCGTTCACGGCGTC
GACCTCTTCGACGCGGGCTTTTTCGGGATCTCCCCGCGCGAGGCGCAGCCGATGGATCCC
CAGCAGCGGCTGGTGCTGGAGACGGCGTGGGAGGCGCTGGAGCGGGCCGGCGTGCGCCCC
TCGGCGCTGAGCGGGAGCGCCACCGGGGTATACCTGGGCTCGGCCGGCTCGGATTACGGC
AGCCAGATCGGGAGCGCCCTGGATGCTCTGGACGGGTACCAGATGACGGGGAATCTTGGC
TCCGTGATCTCTGGCCGGGTGGCTTACGTGCTGGGGCTTCAGGGCCCAGCGATCACGGTG
GACACGGCGTGCTCGTCGTCGCTGGTGTCGCTGCACCTGGCGTGCACGGCGCTGCGCCAG
GGCGAATGCGACCTGGCGCTGGCCGGCGGGGTGACGGTGATGAGCACCCCCGCGCTGTTC
GTGGAGTTCAGCCGTCTCAAGGGGATGGCCCGAGACGGTCGGTGCAAGAGCTTCTCGGCG
CAGGCGGACGGCGCGGGCTGGGCCGAGGGTTGTGGGATGCTGTTGCTGAAGCGGCTGTCT
GACGCGCGGCGCGACGGCGACCGTGTGCTGGGGGTGATCCGTGGCTCTGCGGTGAACCAG
GACGGTCGCAGCCAGGGTCTGACGGCGCCGAACGGCCCTGCCCAGCAGCGGGTGATCCGG
CAGGCCCTGTCGTCGTGCCGGCTGTCGCCCGAGGACATCGACGCGATGGAGGCGCACGGG
ACAGGCACGAGCCTTGGAGACCCGATCGAGGCCGGAGCGCTGGCGGAGGTGTTTGGACCG
GAGCGTAGCCCCGAGCGTCCGCTGTACCTGGGTTCGTCGAAGTCGAACCTGGGGCACACG
GGACCTGCCGCGGGCGTGGCGGGCGTGATCAAGATGGTGCTGGCGCTGCAGCGCGAGGTG
CTGCCGAAGACGCTGCACGCGGAGCAGCCGAGCCCGCACATCGCATGGGAGGGGAGCGGG
CTGTCATTGCTGCAAGAGGCGCGTCCGTGGCGGCGCAACGGCCGGGTCCGTCGTGCCGGC
GTGTCGTCGTTCGGGATCAGCGGGACGAACGCGCATATCATCCTCGAAGAAGCGCCGGCC
GAGGCGCGGCGTGCGCCCGTCGAGGTCAAGGCCGAGGTGGCGCCGGCGGCGATGCCGCTG
GTGCTGTCGGGTCGAGACGAGGCTGCGGTGAATGCGCAGGCCGGGCGGTGGGCGAAGTGG
CTGGAAGGGCACGCGGAGGTGGGGTGGTCGGACGTGGTGCGCACGGCAGCGCTGCACCGG
ACGCACTTCGCCTCTCGCGCATCGGTGCAGGCGGCGAGCGTGTCCGAGGCGGTGGAGGTG
CTGCGGGCGCTGTGGCAGGGTCGCGGCCACCGGGCGGTGTCGGCGGGCACGGCGCGTGCG
CGAGGCAAGGTGGTGTTCGTGTTCCCCGGCCAGGGGAGCCAGTGGCCGGGGATGGGCCGA
GCGCTGCTGGAGCAGAGCGCAGCGTTCGCGGAGGCGGTGCAGGCGTGCGATGAGGCGCTG
CGGCCGTGGACGGGCTGGTCTGTGCTGTCGGTGCTGCGCGGAGATGGCGGGGAGGAGCAG
CCGTCGCTGGAGCGGGTGGACGTGGTGCAGCCCGCGCTGTTCGCGATGTGCGTGGGTCTG
GCCGCGGCGTGGCGGTCGCTGGGGCTGGAGCCTGCGGCGGTGGTGGGCCACAGCCAGGGC
GAGGTGTCGGCGGCGGTGGTGTGCGGAGCGCTGTCGCTTGCGGAGGGAGCGCGGGTAGTG
GCGCTGCGCAGCCAGGCGGTGCGGCAGCAGTCGGGGATGGGGGCGATGATGCTGGTCGAG
CGGCCGGTGTCGGAGGTGCAGGAGCACATCGCGCCGTACGGGGAGGCGCTTGCGATAGCG
GCGGTGAACACGTCGAGCTCGACGGTGGTGTCGGGTGACGTGGAGGCGGTGGACGGGCTG
ATGGTGGAGCTGACGGCAGAAGGTGTGTTCTGCCGGAAGGTGAACGTCGACTACGCGTCG
CACAGCGCGCACATGGATGCGCTGCTGCCCGAGCTAGGAGCGAAGCTGTCGTCGCTAAGG
CCGAAGGCGACGCAGCTGCCGTTTTACTCGACGGTGACAGGAGAGGTGTCGCGGGGCGAG
GCGCTGGACGGCGAGTACTGGTGCCGCAACCTTCGGCAGACGGTGCGCCTGGACCGAGCG
CTGTCGAAGCTGCTGGAGGACGGGCACGGTGTGTTCGTGGAGGTGAGCGCGCACCCGGTG
CTGGCGATGCCGCTGACGACGGCGTGCGGGGAGGCGCAGGGGGTGGTGGTGGGGAGCCTG
CAGCGCGACGAAGGTGGGTTGTCGCAGCTGTACAGGACGCTGGGGCAGCTGCACGTGCAG
GGGCACGAGGTGGACTGGGCACGGGTGCTGTCGGGCCACGGCGGTGGTGTCGTGGAGCTG
CCGACGTACGCGTTCCAGCGGCAGCGCTACTGGCTGGATATCTCGAAGGCGCGTAGCGAC
GTGAGCTCGGCGGGGCTGAAGGCGGCCGCCCATCCGCTGCTGGGAGCGGCAACGAAGCTG
GCTGAGGGCGATGGCCATCTGTTCACCGGCCGGCTGTCGCTGGGCGAGCATGCGTGGCTC
CGCGACCATGAGGTGTTTGGTAACGTGGTGTTCCCCGGCGCGGGGATGCTGGAGCTTGCG
CTGGCGGCTGGGCGCACGGTGGGCAGCGGGGCGCTGTCGGAGATGGTCTTGGCCGAGCCG
CTGGTGCTCGCCGAGGACGTGGCCGTGCGACTGCAGCTGTCGGTCGGCGCGCCGGACGCG
GCGGGCCGGCGTGAGTTTGGGCTGTACAGCCAGCTGGAGCAGGGGCCAGAAGACGCCCCG
TGGGTGCAGCACGCGACGGGGGTGTTGACGGACGAGCCCCGCGGCATCCCTGGTGAGCTC
GATGAGCTCGCGACGTGGCCAGTGCCCGGCGCGGAGGCGGTGGATCTCTCCGGGTTCTAC
GAGCGGCTGCGTGAGCGTGGACTCCACTACGGCCCGGCCTTCCAGGGGCTCGTGGAGCTG
TGGCGTCGAGGCACGACGTTGTTCGGCCGCGTGGTGTTGCCCAAGGCCGCGGGGGACAGC
GCCGAGGACTACGGCGTGCATCCGGCGCTGATGGACGCCGCGCTGCATACGATGGTTGCG
GCCCTCTCCGAGAGGCCAGGAGCGAACGCGGTGCTCCTGCCGTTCGCTTGGTCGGACGTG
GTGTTGCTCGCCATGGGGGCGAGCGAGCTCCGGGTCCGGATGGAGCTCCAGGAGACCGCG
GACTCCAGGCAGATCACGGCTTCGTTGTCCGTCGCAGATGCGATCGGCCAGCCCGCGGCG
AGCGTCGGCGAGCTGCAGCTGCGCTGGGCGACAGCCGAGCAGCTGAGGGCGGCGATCCGT
ACCGAGGCGCAGCATCTGTATCGGGTGGACTTCCGCCCTGTGCGCCTCGTGGCCGGCTCG
TCGGAGCCGGCCCGGCCAACGCTTGTCATGGTAGCGGAGGGGCAAGGGCCACTGGCCGAG
ATCCTGGAGGCGGAGGCGGTGGCGAGCCTCGATATATTGCTCGCACGTCTCGAGCAGGGC
GCGAGCGCGCCTGTGCGGGTGGTGGTCGATGTCACTGCTGCCGCTACCAGCCAGCCGGGC
GTGCCGGCGGCGTCGCACGAGGCGACGCAGGAGGCGCTGTCGTTGCTGCAAGCGTGGCTG
TCGCAGCCCCGGCTCGAGGGGGTGGAGCTGGTGTGGGTGACGCGAGATGCGGTCAGCGCC
GCTCCGGGCGACGGTGTCCAGGACCTGGCACACGCGCCGCTGTGGGGGCTTGTTCGCACG
GCGCGAAGCGAGCACCCCGAGCGCCAACTGCGCCTGATCGACGTCGGGACCGAGCCTGTG
GATGGCGAGCTGCTGGCGCGCGCGCTGGCGACGGCGACGGAGCCGGAGCTTGCGCTGCGC
GGCGGCGCTGCGCTGGCCGCGCGCCTGGTGCGCGTGCCGGCGGCAGCGGAAACGCTTACG
CCGGCGCGCGGGCTGGACCGGACGGGCACGGTCCTGGTGACCGGGGGAACAGGCGAGCTG
GGTCAGGCCGTTGCGGAGCATCTGGTGCGCGCGCATGGAGTGCGGCACCTTGTGCTGACG
TCGCGCCGTGGGCTGGAGGCGCCCGGGGCCCGGGAGCTCCTGCAATCGCTGGAGAAGCTC
GGCGCCGAGACCGTGACGGTGGCGGCGTGTGACGTGTCGAAGCGAGAGGAGGTCGCGCAG
GTGCTGGCCGGCATCGAGGCCGCGCACCCGCTGACCGCGGTGCTGCACCTGGCCGGCGTG
CTCGACGACGGCGTCCTGTCGTCGCAGACGCCGGAGCGCATTTCACGGGCGTTCGCGCCG
AAGGTGGACGGGGCGCTGCACCTGCACGAGCTGACGCGGGAGCTCGATCTGTCGGCGTTC
GTGTTGTTCTCCTCGATGTCCGGGACGCTCGGGACGTCGGGCCAGAGCAACTACGCGGCG
GCCAACAGCTTCCTCGACGCGCTCGCGGCGCACCGCCGCGGCTGCGGGCTCGCGGCGACG
AGCCTGGCGTGGGGCTTCTGGGCGCAAGCGGGTGTGGGCATGACAGCGCACCTGGGCGAG
GCAGAGCTGTCACGCATCCGGCGCGCAGGGCTTGTGCCGATGTCGATTGAGGAGGGCCTT
TCGCTGCTGCATGCCGCGCTCCTGCGCGCCGAGGCGAGCCTGGCCCCGGTGCGCTTCGAT
CTGGCGACGCTGCAGCGCAAGCTCGACACCGGCGGCGCGCTGCCCCCGCTGTTCCTGGCC
TTGCTCCGCCCGGGCCTGCGCCGGGTTTCCCCGGCATCACAGGGCACCTCGGCGATTCGC
GAGCGTCTCGTGGCGCTGCCTGAGCAGGAGCGGTTGAAGTCGCTCGTCGCTCTGGTTCAA
GCCGAGGTCGCCGCGGTGCTCGGGTTGCAGGGAGCGGCTACCATCCGGGCCGACCAGCCC
CTGCTGGAGCTCGGAATGGACTCCTTGATGGCGGTGGAGCTTCGAAACCGGCTCTCCAGC
CTGATCGGCGCCACGCTCCCCGTCACCGTGGCCTTCACACATCCAGACTCCAGGTCCATC
GGTGCTTTCCTCCTCGACATGCTCCCTTCGGCCGGGGAGCGCTCGCTCCCGCGGAATGAC
AAGGTCGCCGGCAAGTGGCTTCGCATCCTCAGGTCGCCGCGCGTCCCCGCCGTCCGGATC
GTCTGTTTCCCGGGCGCTGGAGGGGCAGCCTCTCTGTTCTTACCCCTCGCCCAGCACGTG
GCCGATGACGTGGAGCTCGTGGCGATCCAGGCCCCGGCTCGGGGTGATCGGCTCGCAGAG
ACGTCGGTCACGGACATGAGCGTTTTCGTCAGCGAGGTGTGCGACGCGCTGCGCGGTCGA
CTCGACTTGCCCACGCTGTTCTTCGGCTACAGCTTCGGGACCTGGACAGCTTACGCGGTG
CTGTGCGGCGCCTTGTCTCGTGGCGTGCATCGTGCGCCGCTCGGCCTCGCCGTCGCCTGC
ATGACCCCGCCGTCCGACGCACGACAGCGCACGATGCAACTGGGGATCGAGGAGGACGAC
GACACCGTCGTGAAGAGAATGGTCGCCGTCGGGGCATGGCCGGAGGCAGCCCTGGACGAC
GGCGAGCTTCGTGCGGCGCTCCTCCCCTCGTTCCGTGCGGATGCGCGGCTTGGAATGAGC
TATCGGTGGGCCGAGGAGAAGCTGCTGGACGTCCCTGTCCTCGCCGTGGCAGCGACGAGG
GACGAGCTATCCCAGATGCGTCATCCATCGAGGCGTGGCGTCAGGTGACGACAGGAGACT
TTGTCATGAGCCACCTCGACGGCACGCACTCGCTCGTCCTGGATGATCCTGCGAGCCTGG
CACGAGAGCTGTCGACGTGCGTGGAGACGCAGCTGTGGCCAGGGAGAGAGCGCTCGAGCG
ACCGCGCCACCCAGGCACCCGGCGACAGCCGCGGGGACGGCGCGGCGGCCGGTGTCGACG
GCATGCCGCCGCTCGGAGAAGAGCGCGAGACACCGGGCACCAGCCCATCGCAAGCTTACG
TGGGCTGATCGTCGCGTCGCCCGCATCTCCAAAAACGGTGCCCCAGCGCTTGGAGGCAAG
ATTGGTCGTGATCACGACGGAGCGGCTCTCGGCTAGCGCGCGAGGAAGCCGCGGATCGCC
GCGTTGACGATCTCGGGCTCCTCGTGGACGATCCAGTGGGTCGCCTCGGGCACGCGCATC
ACCCTCAGATCGGGGACGTAAAGCTCGATCCCGGCGAGGTTGCTCGCGGGCAAGAGGTAC
GGATCGCGCTCGCCCCAGATCACGAGGACAGGCACGGAGACGGTGAGCGAGCGGAGACCC
CGGGTGAGGTTGCTCCCCCCCACGGGCTGGCCCGGGCCTGGAGGCCCGATCTGCGCCGCG
CGGTAGATGTTGAGCCCGCCCGTGATCGCGCCCGGCTGCGCGAACGCCTCCAGGTAGACA
CGGACGTCCGCTTCGCTGAAATATCCCTGCGACAGCCCTGGCTCGAGGAAGATCTCCCGC
AGAAAGCCGTACTCGTTCCGGGAGAGGTGCTCCTCTGCGGACCGGCTCCGGAAGAGGTGC
ATGTACTGGCTCGCGGCCTGCTGCGACGGGTCCTCCCGCAGCGCGCGGTCGAAGCACGCC
GGGTGAGACATGTTGATGGCGACGAGCTTCTCGACGCGCTCCGGGTGGCGGAGCGCCACG
GCCCAGGCCACGACCGCGCCCACGTCGTGGCCGACGAGCACGGCCTTCTCCTCGCCGAGG
TGCTCGATCAGCGCCGCGATGTCCGCGGCCAGGAGGTCGATGCCGTAGGCGTCCACGGCC
GACGGCTTCGACGACAGGTTGTAGCCGCGCTGGTCCAGGGCCACCGCGCGGTGGTGGCGG
CCGAAATCGAAGAGCTGCCGCTTCCAGGCGTACCAGAGCTCGGGGAAGCCGTGGATGAAC
AGGATGAGCTTGCCCGCGCCCCTGGACGCGTAATGCAGGCGGACGCCGTTCACGTCGGCA
TGAGCATGGATGATGTCGTTCCTTGGCAGCATGGCGATGACCTCACCCGAGTCCGAGCCG
CGCCGGCCCTGCGCTCGACGGCGCGCGCGCGCGCCCGGCGCGCGGCGCCGCCGGCTCCTC
GCGGCGAGCCCCGTTCCTCTCGTGGCGGAGCTTCGATATCGCCTCGTAGACCGCGCGCCG
GGCGCGGTTCAGCCCGCCGAGCGGCCGGTGCGCCGGCAGGGCGTGCCAGGGCGTGAACGA
GAGGTTCTCCTCGAACTCCGCCTGTCCCGGGAGGTCGAGGCGCTGCGCCGGGATGACCAT
CGTCGCGACCTTGGTGAACGCCGACAGTTCCTCGCTCCAGCGAATCGTCGGATCCTCGAT
GGGCGTCTTCGTCGGGTGGGCCTGGAGCTGCACGAGGAAGTCGAACATCCAGTCTCCCCC
CTCGAGCTGCCGCGCCACCGCCTGCTTGAGCGCGTCGTCGCCCGGCTCGACCCCGACGCC
GGGCGCGCCGCAGGGCCGGGGGACCACGCAGAACTTCATCGCCCGCGCGCCGAGCCGGAA
CGGCGTCTGGCTGAAATACCGGGCGTGCAGGGGGTTCTTCACCGGCTTGAGCACGACGGA
GAGGTAGTTCATGAGCTCGCGCAGCCGGAGGCGGGGCGGGCGGAGCGAGCAGAAATACGA
GAGGGGCTTGCCCGCGCTCATGGCAGACATGAGCTCGACGTAGTCCGCGATGTTCCGCGA
GAAGAACACGTCCGTGTTCCCCATCAGGAAATCCTGCGTCGTCGCGTCCTTCTCGCCGTC
GAGGATCTTCTCCCCCTCGACGCCGAGCAGCTTGATCGCCATGCAGCGGGAGTCOCGCTT
CATGTCCGACTGGACGCGCGAGGAGGTCGACGAGAACCGGACCCACGCCGGGTAGACGCG
CGGCTCCCGGAAGACGCCATGGCGGAGCTCGCGGGGCAAGCCCTCGTCGACGACGAAGTG
GGCCCTCACGCAGCCGTGCGACTTCGGGTGCACCCCGCGGAGCGCGGGCGAGGCGCCCGC
GGGGTAAGCTTGCTCGTAATTGCGCCTCATGATCTCGGTGATCTGGCGGATGCTGTCTTC
CTCGCCGGGCGGCGGGTACTCCTCGTGGAGCTTGAGCGTCGTGTTCATGACCAAGATCCT
CGGTGTCGTTGGGGGGTCAGGAAGGCTTGGCGACGGCGCGCTCGCGCGCGGGGAACGGCA
GGGCGGTGCTCGCGACCTTCACGCCGGACGCGCTCTCCTGGCGCTTCCGGTGCTGCACCT
CGTTGCGCATCTGGATGAGCCGAGCGAACGTGGTGACCCAGAAGGGCGCTCCCAGGGAGA
GCAACCCGCACGAGAAGGCGAGCCCGGCCAGCCAGAACAGCGTGTCCTGGAGGAGCTCCA
GCGAGGTCGGCGGGGGCGCCAGGATATCGTGGCGGTACGCGCGGAACCGCTGGACGATCC
AGCTCTGCTGCCAGCCGACGCCGAGGTTCGCCTCGTCGAGGAGGGTCGCCGTCTTCTGAA
TCTCGAGCCCGAGCTCCTCGAGCGTGGCGTCGGGGAGCTCGGCCGGCGCGCCGGCCGGGA
GCTCGCCCAGGCGCGCCGCGGTGGTCGCCACCGTGTCCGCCTGGGCGGCCACCTGCGCGC
GCGCGGCGCCGCTCTGGTGGAGCCGCGCGAGCACCTGGAAGCCGTCGAAGTTGAAGAGGA
TGACCACCATCATCGAGACGACGAGCGTCAGCCGCCGGACGCGCAGCTTGAACGTCTGCG
ACGCCGTGCCCTCGAGGGTGGGAAACCAGCGGCGCACGTACGTGGAGAACTGCTCCAGCG
TCGCGTAGCGCTTCGGCGCGCTGGAGCCGCCGTGGCCGCAGCTCACCCAGCGGCTCGGCA
AGAGGAGCTCGCGCCCGTCCCCGTCGGCCGGCGCCGAGGCGCCGGTGGCGATGCTGATCA
GGTCCTCGACGTCGATGTACTCGACGAGGCCCGCGAGCCGCTCGGGGTCCGAGCCGTACG
CGAGCTTCGCGCTGCGCGCGTGCAGGACCGGATCGGAGAGCACCTCGTGCACGAAGCGCA
TTTCACAGGCTTCGTTCCTGGCGAGCGCGCCATGCGCCGGGCGCCCGGTCGCCGGATCGA
CGACGTCGGGGTCCTTGTGGAGCGCGCGGAAGCCGTGCATGAGCGACCGCAGCATGTGGA
CGAGGACGCAGGAACGGTAGCGTCCGACGTTGACGATCACCTGCACGACCGCGCTCACGA
ACAGGCTCGCAGTGAGCATGATCCCTTTAGTGAGGGTTAATTGCGGCCGCC

The sequence of each of the ORFs in this gene cluster and the translated amino acid sequence of the proteins encoded thereby are shown below.

orf1 partial sequence bases 522-1
ATGGAGAAGAATCCCTGGCTGTCGCACTGGACCGCGCGGACGGACGCGAAGCTTCGCCTT
TTCTGCTTCCCCTACGCCGGCGCCGGCGGGGCCATATATCGAGACTGGGCGAAGAGCCTA
CCGGCGCATATCGACGTCTGCCCGATAGAGCCTCCGGGGCGATTCGCTCGGTCCAAGGAG
AAGCAACCTCGCTCGATGTCGGAGTTCATCGACGGCATCGAGCGGGGGCTGGATGACATG
CTCGACCTGCCGTTCGCGTGTTTTGGATACAGCCTGGGAGCGCTGATGGCCTTCGAATGG
GCCCGTTCTCTCCGCAGGCGCCGTGGGATCGAGCCGACATGCATGGTCGTCGCCGCGAGC
AGAGCCCCTCACGTACCCGCCCGCCTGCCGCCGATCTCTCACCTCCCCGCGAGCGAGTTC
CTGCGGCGTGTACAGCAGAGGTACGGGGCGTTCGATCGTGTGATCCAGGAGGACCCTGAG
CTGCTGCAGCTCGTCGGCAAGATCATGCAGGTCGATCTG
MEKNPWLSHWTARTDAKLRLFCFPYAGAGGAIYRDWAKSLPAHIDVCPIEPPGRFARSKE
KQPRSMSEFIDGIERGLDDMLDLPFACFGYSLGALMAFEWARSLRRRRGIEPTCMVVAAS
RAPHVPARLPPISHLPASEFLRRVQQRYGAFDRVIQEDPELLQLVGKIMQVD
orf2a bases 791-1144
ATGAACACGCGAAAAATTCGTATCGTACTGTATGCCGCGCTCACGCTGATCGGCGCTGCC
GGCGTTTTGTGGTTCAATATACGGTTCATGAACGCCGGGGGGTTCGATTCACCGACGGGG
TTCGTCGACGCGGCCACGGTCAACGCGGCGGGCGCTTCCGTGTTCGTCGACATCGCGGTG
GCCGCCACGGCTGGGATGCTGTTCATGGTGGTCGAGGGCCGGCGGGTGGGCGTGCGCCAT
GTCTGGGCGTATGTGTTGCTGAGCTTTTTGCTCGCCTTCGCGGCGACGTTCCCGGCGTTC
TTGCTGGCGCGCGAGCTGCGCCTCGCTGCGCTCGAAGGGGCGGGGGGACGCTGA
MNTRKIRIVLYAALTLIGAAGVLWFNIRFMNAGGFDSPTGFVDAATVNAAGASVFVDIAV
AATAGMLFMVXTEGRRVGVRHVWAYVLLSFLLJAFAATFPAFLLARELRLAALEGAGGR
orf2b bases 1233-760
GTGAGCACCGCGCGCGGCCTCCCGGCGCAAGTGGCCGGTCTCTCCGGCGCGCTCCACGTA
GCGCGCCGGGGGCCGCCCCGCCCGGGCCGTCAGCGTCCCCCCGCCCCTTCGAGCGCAGCG
AGGCGCAGCTCGCGCGCCAGCAAGAACGCCGGGAACGTCGCCGCGAGCCGAGCAAAAAAG
CTCAGCAACACATACGCCCAGACATGGCGCACGCCCACCCGCCGGCCCTCGACCACCATG
AACAGCATCCCAGCCGTGGCGGCCACCGCGATGTCGACGAACACGGAAGCGCCCGCCGCG
TTGACCGTGGCCGCGTCGACGAACCCCGTCGGTGAATCGAACCCCCCGGCGTTCATGAAC
CGTATATTGAACCACAAAACGCCGGCAGCGCCGATCAGCGTGAGCGCGGCATACAGTACG
ATACGAATTTTTCGCGTGTTCATGGCGATGTCTCTATGGGAACACCTAACATAG
VSTARGLPAQVAGLSGALHVARRGPPRPGRQRPPAPSSAARRSSRASKNAGNVAAKASKK
LSNTYAQTWRTPTRRPSTTMNSIPAVAATAMSTNTEAPAALTVAASTNPVGESNPPAFMN
RILNHKTPAAPISVSAAYSTIRIFRVFMAMSLWEHLT
orf3 bases 2171-1230
ATGACTCGAATCTCTGGCCATGCGCCAGGGCACCATGAGGACGGGATGGATATACATCGG
AATAGAGCGAACTGGCGCGCGGCGCTGAGCGGGGTGTTCACGTCGGTGCACACCATCGCG
AAGCGCAGGGACCCTCGCTGGTACTACAACAACCTCGGCGAGAACGTATTCACCGAGCAG
TCCAAGTTCTTGAACCTCGGGTACTGGGCCGACCCAGAGACGAGGACGATGGACGCCGCG
TCGACCGCGTTGGTCGACCTCGTCGCAGAGGCCGCGGAGCTCTCGGGGAGCGACGAGGTG
CTGGACGTGGGCTTCGGCTTCGGCGACCAGGACATCCACTGGACAACGCGTTACTCGCCG
AAGCGCATCGTCGGCATCAATGTCACAGAGAAGCAGGTAGAAGAGGCGCGCAGGCGGATC
GCCGAGCTCGGGCTCGACGACCGCATCGACCTGCGCGTGGGCTCGGCGACGGACATGCGC
TTCGCGCCGGAGACCTTCGACAAGGTGCTCGCCGTGGAGTCGGCCTTTCACTTCGATACG
CGCGACAAATTCTTCGGCGAAGCCTTTCGGGTCCTCCGCCCGGGAGGTCGAATCACCACC
GCCGACATCATCCCGCTGCCAGGGCGGGGGAGCTCCCCGCTGTTCACCCTTCGGTCGCAA
GCCCCCGCGGAGAACCACTACCCTCGCGACGTCTATGCGGAGAGGCTTCGCGCCGCCGGC
TTCGTGAACGTCGAGGTCCGGTCGATTCGCAAACACGTCTACGATCAGTACCTCGACCAC
CTGGATCGCCGCTCGGCGAGCCCCGACTTCAAGCACAAGAGCCCGTTGCTCCGGCTGCTG
CTGATGAAGGAGAACCCGCTGCTCTCCTGGCTGTTCGCCTCATTCGATTACGTCATCGCA
TCCGGAGAGAAGCCCGGGAATATGCCCCACTCCGCCAGGTGA
MTRISGHAPGHHEDGMDIHRNPANWRAALSGVFTSVHTIAKGRDPRWYYNNLGENVFTEQ
SKFLNLGYWADPETRTMDAASTALVDLVAEAAKLSGSDEVLDVGFGFGDQDIHWTTRYSP
KRIVGINVTEKQVEEARRRIAEIGLDDRIDLRVGSATDMRFAPETFDKVLAVESAFHFDT
RDKFFGEAFRVLRPGGRITTADIIPLPGRGSSPLFTLRSQAPAENHYPRDVyAERLRAAG
FVNVEVRSIRKHVYDQYLDHLDRRSASPDFKHKSPLLRLLLMKENPLLSWLFASFDYVIA
SGEKPGNMPHSAR
orf 4 bases 3456-2248
ATGCGACGGAGCCTGCACGACCACAAAACGCAAGGAAGACCAATGAGCTCCACGCAATGC
CATCTCAGCATTCACGATATACTCGCCTCAACGCGAGAGCTCGTCCCCGCGCTGCCCGCT
TCTGCAGCCGAGGTCGAGGCAGCCCGCAAGCTCCCCGGTAGCGTATTGAGCGCCCTGGAC
AAGACGGGAGTGTTTCGCATGGCCATGCCCAAGGCGTGGGGCGGTCCGGAGGCCGATCCG
TTCTCGCAAATCGAGCTGATAGAGAACCTCGCGCTCGGCGACGCCTCGTCGGCGTGGTGC
GCGATGATCCTTTCGGGCAGCGGCTTCTACGCATCCCATCTGGAGGATAGCGTAGCGCGG
CGCCTCTACGGCAAGCTCGACGTACGCCACGCCGGAAGCCTGAGGTGGACCGGGAAGGCT
GAGAAGGTGGCCGGAGGCTATCGGGTGACAGGGACCTGGGACTTCTGCAGCGGTTGCCAC
CACGCGGACTTCCTTGCGACGGGCTGTCTGGTCACCGAAAATGACAGCCCGGTTCTCAAC
GCGGACGGCAGCCCGCGTACCCTCTGGGCCGTCCTCCCCGCTGGAAGCTTCGAGATCCTG
GACACCTGGCACACGACGGGCCTGGCGGGAAGCGGTAGCAACGACTGCAGCGCGAGCAAC
GTCTTCGTGCCCGAGGAGCACACCTTCAACCTGGCGGACCGCAAGCGCAGCGAGCCGCTG
TACGCCTATGCCCCCCTCTTCCTGCAAACCCTGGTGGGCGTTCAGCTAGGCATCGCGCGC
CACGCGATCGAGATCGCGCTCGAGCTCGCGCAGAACAAGAAGGTAATGCCGATGGGGCAC
AACCTGGGCACCGAGCAGAGCGTGCAGATAGCGATCGCCGAGGCGGAGGCCACGTTGGGC
TCGGCCAGGAGCTACGTGCGCGATGTCGTCGGCGAGCTGTGGGATGCGCTGCAATCCGGC
AACGGACCGTCGCTGAAGCAGCGCGCCAAAGGAGTGCTGATGATGATCAACGCCGCACAA
TCGACTCGACGCGTGGTCGAATCGATGTGCGACGTGGCAGGGGCCACCGCGATCTTCGCC
AAGCATCCGATCGAGCGCCTCCGGCGGGACATCATCACCGCAAACTCGCACTTCCTCTGC
CAGAAGAATAGATACTCAATCATCGGCCAGGCGCTGATGGGTATCGAGCCGGCCTACCCC
TACTTCTGA
MRRSLHDHKTQGRPMSSTQCHLSIHDILASTRELVPALPASAAEVEAARKLPGSVLSALD
KTGVFRMAMPKAWGGPEADPFSQIEVTENLALGDASSAWCAMILSGSGFYASHLEDSVAR
RLYGKLDVRHAGSLRWTGKAEKVAGGYRVTGTWDFCSGCHHADFLATGCLVTENDSPVLN
ADGSPRTLWAVLPAGSFEILDTWHTTGLAGSGSNDCSASNVFVPEEHTFNLADRKRSEPL
YAYAPLFLQTLVGVQLGIARHAIEIALELAQNKKVMPMGHNLGTEQSVQIATAEAEATLG
SARSYVRDVVGELWDALQSGNGPSLKQPAKGVLMMINAAQSTRRVVESMCDVAGATAIFA
KHPIERLRRDIITANSHFLCQKNRYSIIGQALMGIEPAYPYF
tmbA bases 3853-31557
GTGGCGCCGATGGACAGAATGCTTGGGTTTCTGCGCGATGCATTGGCGGATCTGCTGCAG
GTTGCGCCGGGAGCCGTTCAGGATGATGTTCGATTCCACGAGCAGGGCCTCGACTCCGCG
AGGGCGCTGCTCCTGGTTGAAAAGCTGTCGAGCTGGACGGGGCGCGCTCTCCCAGCCACG
CTGGTATGGCAGCACAGCACCCTGAACGCGCTTGCTAGGCATCTGGCGGATACAAGCGGG
GAGCCCATTTCGACGGCCCTCGGCGCGCGCGCCATCGCGTCGTCGTCCATCGACGAGCCC
GTCGCCATCGTCGGGCTCGGGTGCCGCATGCCTGGCGGCATCGCGTCCCCCGAGGGGATG
TGGCGGCCCCTGTGCTCGGGCGTGGATGCCATCGTCGAGGTTCCTCCGGACCGCTGGGAC
GCTGAGGCCTGGCTGGACGCGGACGCCCAGGCGCCCGGGAAGATGACGACCCGATGGGGT
GGTTTTCTCAATGAGATCGTGAGCTTCGACGCAGGGTTCTTCGGAATCTCGCCGGCTGAA
GCCAGGCAGATGGATCCGCAGCAGCGCTTCGCGCTCGAGACTGCCTGGGCAGCGCTCGAG
GACGCGCGCGTCGTTCCCACCGCGCTCGGGGGGTCTCGCACGGGCGTGTTCTTCGGCGCC
ATGTGGCAGGAGTATGCCCGCGTCGCGGGGGCGGACGCGACAGGCATAGAGCCACACTCC
GCCGTGGGCTGGGATAACTCCATCATCCCCGCCCGCATCGCCTACGCTCTGGGGCTTCGC
GGGCCGGTGATGACGGTCAACACGGCGTGCAGCTCCTCGCTGGTAGCCGTGCATCTCGCG
GCGCTGAGCCTCAGGCGTGGGGAGGCGGATCTGGCGCTTGCGGGCGGGGTCAACCTCATG
CTGACCCCCCACGCCACGGTCCAGATGACCAAGTTCGGCGCGATGAGCCCTGACGGCCGC
TGCCGGGCTTTCGCCGCAGGAGCCAACGGCTACGTCCQTGGCGAGGGGTGCGGCGTGGTG
GTGCTGCGCCGCCTCTCGGACGCGCTCGTCGCAGGCGATCGCATCTACGCGGTGCTGCGA
GGCAGCGCCGTGAACAGCGACGGCGCGTCCAATGGACTCACGGCACCGAACCCTGAGGCC
CAGGCTGACGTCATCCGAGACGCCTGGCAGCGAGCTGGGGTTGCGCCCCGGGAGGTCGCA
TACGTCGAGACCCATGGTACGGGCACGATCCTCGGCGATCCTATCGAGGCCGCGGGGCTG
GCCGCCGTGTTCGCGCCCGGCCGGGAGCAGCCGCTCCGCATCGGCTCGGTAAAGACGAAC
TTCGGCCACCTCGAGGCTGCGGCGGGCATCGCCGGGCTCCTCAAGGCGACGCTCGCGTTG
CATCACGGGCAGCTGCCGCCGAACCTGCATTTCGAGAGCCCGAACCCCCACATCGACTTT
CAGGCGCTCCGGCTCGAGGTTGTCGCACGGCAGCAAGGCTGGCCGAGGGACGCGGGCCGT
CTTGCCGGCGTCAGCAGCTTCGGCTTCGGCGGCACGAACGCCCATGTTGCGCTGGAGGAG
TCTCCCTACAGGCCGCAGATCCTCGTCCCCTTGGCTGCTGATGGGGAGCAGGCGCTCCGC
GATGCGGCGCGACGCCTCCTGGCCGATTTCGCTGCTTCGGGCGCGGGTCGCGATGCGGAG
CGGCTCGTGGTGCAGCCCGGCACCGGCTCCGTTCGCGCCATGGTCCAGGGGCGCGATGGC
GCCTCGGTGGAGGCTGCATTGCGGGAGCTGGCCGACGGTGGCGCGGAGCTCCGCCCGATT
TCCCGGCCGAGGCCGCGGCTGGTGTTCTTCTTTTCAGGACACGGCGGCCACTGGGCGGGC
ATGGTCCGTGACCTGATGGCCGGGGAGCCGGTGTTCCGCGCGACCCTCGAGGCCTGCGAT
CGAGCCGTCCGGCAGTTCACCGGGTGGTCCGTGATCGCCGAGCTCGCGGCGAACGAGGCG
TGCTCGCGGCTCGACCGAACCGACGTCGTGCAGCCCGTGGTCTTCAGCGTTCAGGTCGCG
CTTGCTCGCACCCTGCAAAGCTGGGGCGTGGTCCCTGAGCTCGTTTTCGGCCAGAGCGTT
GGCGAGGTCGCTGCGGCCGTGGTCGCGGGAGCCCTGTCGTTGCGCGACGGGGCGCGGGTG
ATCTCCACCTGGTCGGCTCTGATCGCGAATCATGCGTCAGGACGTGGCGCGATGATCGTC
GCCGATCTGTCGGTCGCGCAGGCCGAGCGCGTCCTCGCCAGCTGGGACGCGGGGGGCGTT
CAGGTCGCCGTCCATCTGGCCCCCGGCCAGGTTTGCCTGGCCGGTCCGGTCGATGCCGTC
GAGCGTCTTGTGCGCCGGTTCGCGAGCGATGGCATCGGCACCATGCGCGTGCGCATCGAC
TACGCCGCTCATACCGCCGAGATGGAGCCGTTGCTCCCCGAGATCGAGCGGCAGCTCGCG
GCGATCCAGCCCAGCGCGCCCACGATCCCGATGTGGTCCACGGTCGCGAACCGGTATGTC
GCCGGTCCGGAGCTCGATGGACACTACTGGGCGCGGAACCTCCGCGAGCCGGTTCAGCTC
GTCGAAGCTGTCGAGAGCGTGGCGCGGCAGCACAGCGCGTGCATCGTCGAGGTGGGGCCG
CATCCGGTCGCGGTGCGCTCGTTTGAAGCGTCGCTGAAGACGTGGGGAGACAGCCGTTCT
GTGGTCCTCGCGACCTGCTGGCGCGAGGTGGCCGCGCGGGGCGGGCTGGAGGCGCTGCTC
GGCGCGTTGTGGCGCGAAGGGATCGATGTCGACTGGGACGCGGTCCGCACGGGTGGCCGC
GCGGCGACTCCGCGCGCGGTCACTCTCCCGCTGGTGGTGTCCGGGAAGACGGTGCAGGCC
CTGCAGGAGAACGCCGCGCGCCTGGCGACACATCTTGATGGCCGAGGCGACATGGCTCTC
GTCGACGTCGCCTTCACCCTGGCGACCGCTCGCGCGCACTTCGCCACGCGGGCGTCGGTG
CAGGCGGCGAGCGTGTCGGAGGCGGTGGAGGGGCTGAGGGCGCTGTCGGAGGGTCGAGCC
GCGGCAGGTGTGGTGCGCGGGACGGGAGTGCACGGGGGGAAGCTGGCGGTGCTGTTCACG
GGGCAGGGCAGCCAGCGGCTCGGGATGGGGAAGAGTCTTTACGAAGCGTGCCCCGTGTTC
CGTGCGGCGTTCGACGAGGTGTGCGAGGCGCTGGACGCGCATCTCGACCGTGGGTTGAGA
GAGGTGGTGTTCGCGGAAGCGGGCAGCGAGCAGGAGGCGCTGCTGGAGCGGACGGAGTAC
ACGCAGCCCGGGTTGTTTGCGCTGGAAGTGGCGCTGTACCGTCAGTGGGAGTCGTGGGGG
CTGAAGCCCGCTGCGCTGCTGGGGCACTCGATAGGAGAGCTGAGCGCTGCGCATGTGTCG
GGCGTGCTGAGCCTTGCGGACGCTGCGAAGCTAGTGTGCGCCCGCGGTCGGCTGATGCAG
AGGTGCCAGGCGGGCGGAGCGATGATGTCGGTGGAGGCCTCGGAGCCGGAGGTGCAGGGG
GCGCTGTCGGCGATGGGGCTGGAGGGCGTGCTCGGGGTCGCGGGCATCPACGGTCCGAGC
CAGACGGTGCTGAGCGGGGACGAAGCGGCGGTGCTGGAGGTGGGGAGGCGGTTCGAGGCG
CAGGGCCGGCGCACGCGGCGTCTGCGCGTGTCGCACGCGTTCCACAGCCCGCACATGGAC
GGGATGCTGGAAGAGTTCGGGAAGGTGGCGCGCGAGTGCGCGTATGGGATGCCGCAGGTG
CCCGTGGTGAGCAGCGTGACGGGCGAGCTGTGGAGCGAGCAGGAGCTGAGGTCACCCGAG
TACTGGGTGAGGCAGGCGCGAGAGGCGGTGAGGTTCTTCGACGGGATGCGCACGCTTGCG
GCGGCGGGGGTGAGCACATACGTCGAGTGCGGTCCCGACGGCGTGCTGTGCGCGCTGGGG
GCGGGGTGCCTGCCGGAGGGAGCCGAGGCGACGTTTGTGGCGAGCCTGCGGCGAGAGCAG
GAGGAAGAGCGCGCGCTTGCAACGGCGGTGGCGACGGTGCACGTGCAAGGGCACGAGGTG
GACTGGGCCCAGGTGCTGTCGGGCCATGGCGGCCGGCCCGTGGAGCTGCCGACGTACGCA
TTCCAGCGGCAGCGCTACTGGCTGGAGGCGCCGAGGGCGCGCGGTGACGTGGGCTCGGCG
GGGCTGAAGGCGGCCGCCCATCCGCTGCTCGGCGCCGCGACGAAGCTCGCCGACGGCGAG
GGGCACCTGTTCACAGGGAGGCTGTCGCTGGCGGAGCATGCGTGGCTTCGGGATCATCAG
GTGTTTGGCAGGGTGGTGTTTCCGGGCACGGGGATGCTGGAGCTGGCGCTCGCGGCGGGA
CGCGCGGTGGGCAGCCGGTCGCTGTCGGAGCTCACGCTCGCCGAGCCGCTGGTGCTGGCC
GAGGAGGGCGCGACGCGGCTGCAGCTGTCGGTCGGAGCGCCGGACACGGCGGGCCGGCGC
GAGGTGGGGCTGTACAGCCAGTCTGAGCAGGCCCCGGAGGACGCCCCGTGGGTGCAGCAC
GCGACGGGGGTGTTGACGGACGAGACCCCCGGCGCTCCTGGCGAGCTCGACGAGCTGTCG
ACGTGGCCTGTGCCGGGCGCGGAGGAGGTGGACCTGTCCGGGGTTTACGAGCGGCTGCGT
GAGCGCGGGCTCGACTACGGTCCGGCGTTCCAGGGCCTCGTGGAGCTCTGGCGTCGAGGC
ACGAGGCTTTACGGCCGGGTGGTGTTGCCCGAGAGCGCGATGGACAGCGCCGAGGCGTAT
GGGGTGCACCCGGCGCTGATGGATGCCGCGCTGCATGCGCTGTTCGCGGCCTCTTCGGAG
GCGGCTGGAGCGGACATGGCGCTGCTGCCCTTCTCGTGGTCGGACGTGGCGCTGCACGCG
ACGGGACCGCGCGAGCTCCGGGTGATGCTGGAGCTTGAAGGCGACATGACGCAAGCGGTG
GCGTCGCTGTGGTTCGCGGACGCGGCGGGCCAGGCTGTGGCCAGCGTCGGCGCCCTGCAT
CTGCGGCGAGCGACAGCGGAGCAGCTGGCAGTGGCCAGCCGTGCCAATGCCCAGCAGCTG
TACCGGGTGGACTTCCAACCGGTGAGCGTGGTGGCGAGCGTCCCGGAGGCGGGCTCGCTG
GTGGTCGTCGGTGCACCGGAGGGAGGAGCGCGGCTGGCCGAAGCCCTGGGGGCGGAGGCG
ATTGCCGATCTCGATGCATTGGTTGCGCGCCTCGAGCAGGGCGCGAGCGCGCCTGAGCGG
GTGGTGGTCGACGTCACCGCCGCGAGCCCGAGCCCGTTGGACGTGGCGGTGTCGTCGCAT
GAGGCGACGAGGCAGGCGCTGTCGCTGCTGCAAGCGTGGCTGTCGGAGCCACGGCTCGAT
GCTGTCGAGCTGGTGTGGGTGACGCGGGGCGCGGTGGGCGCGGCGCCGGACGACGCCGTC
CAGGATCTGGCGCGCGCGCCGCTGTGGGGGCTTGTTCGCGCGGCGCGCAGCGAGCACCCC
GAGCGCCGGCTGCGCTTGATCGATGTGGGGACCGAGCCCGTGGACGCTGGGCTGCTGGCG
CGGGCGCTGGCGATGGCGGCGGAGCCGGAGCTTGCGCTGCGCGGGGGCGCTGCGCTGGCC
GCGCGCCTGGTGCGCGTACAGGCGGCAGCGGAAAGACTCACGCAACCCCGCGGGCTGGGC
CCCGCGGGCACGGTGCTGGTGACCGGAGGCGTGGGCGGCCTGGGTCAGATCGTGGCGACG
CACCTGGTGCGCGCGCACGGGGTGAGGCACCTTGTGCTGAOGTCGCGCCGGGGGCTGGAG
GCGCCCGGGGCCCGCGAGCTTGTGCGGTCGCTCGCGGAGCTCGGCGCCGAGACGGTGACG
GTGGCTGCGTGCGACGTGTCGAAGCGGGAGGAGGTCGCGCGTGTGCTGGCCGGCATCGAC
GCGGCGCGCCCGCTGAGCGCGGTGCTGCACCTGGCCGGCGTGCTCGACGACGGCGTGCTC
GCCGCCCAGACGGCCGAGCGCCTCTCGCGGGTGCTGGCGCCGAAGGTGGATGGGGCGCTG
CACCTGCACGAGCTGACGCGGGAGCTGGATCTCGCGGCGT
TCGTGCTGTTCTCGTCGGTGGCCGGCACGTTTGGCACGGCGGGCCAGAGCAACTACGCGG
CGGCGAACACGTTCCTCGACGCGCTCGCGGCGCACCGGCGCGGCCGTGGGCTCGCGGCGA
CGAGCCTCGCCTGGGGCTTCTGGGCGCCGGGTGCCGGGGGCATGACGGACACCCTGGGCG
AGGCCGAGCTGTCGCGCATCAGGCGCGCAGGATTTATCCCGATGTACGTAGCAGAGGGCT
TGTTCCTGCTGGACGTCGCGCTCTCGCGCTCTGAAGCCAGCCTGGTTCCGGCGCACTTCG
ATCTGGGGAAGCTGCGATCGGGGCTTGACGCCAGCGGTGGAGTGCCCGCGCTGCTCCGGG
CTCTCGTGCGCCCGAGCCTGCGCAAGGCATCCTCCGGGACACAGGAGACCTCGGCGCTTC
GGGCGCGCCTCTCGGCGCTGCCGGAGTCGGCGCGGCTCGAAGCGCTGATCGACCTGGTGC
GGGGCGAGGTCGCCGCGGTGCTCGGGCTGCATCACCCCGCGTTGGTGGAGCCCCACCAGG
TGCTGAAGGAGGTTGGGCTCGACTCGTTGATGGCGGTGGAGCTGCGCAGTCGGCTGAGCT
CCCGGCCCGAGACTTCGCTGCCGGCGACCCTGGCCTTCGACCACCCGACGCCGCGGGCCA
TCGCAGAGCTGCTCCTGAAGCAGGCCTTCTCGGAGCTACAGGAGACGGGGGGCGGCGCGC
GTGTGCGCCGTCGGGTGAGGGAGGACGAGCCGATCGCGATCGTGTCGATGGCGTGCCGGC
TGCCGGGTGGCATCGAGACGCCGGAGGATTTCTGGCGCGTCCTCCGTGAGGGGAAGGATG
TCATCGAGAGCTTCCCCTCCCGATATGAAGCTTCTTCTGTTTACGATCCCGATCCGGAGG
CGGTGGGCAAGAGCTACGCGCGCGAGGGGGGATTTCTACGGAACGTAGACCTGTTCGACG
CGGGGTTCTTCGGGATATCGCCGCGCGAAGCGCAGTCGATGGATCCCCAGCAGCGGCTGG
TGCTGGAGACGGCGTGGGAGGCGCTGGAGCGGGCCGGCGTGCGCCCATCGGTGCTGAGCG
AGAGCGCTACCGGGGTGTACCTGGGCTGGATGGGCTCGGACTACGCCGCTGGCCACGCGG
ACGACCTGAGGACGCTCGATGGCTATCGCATCACGGGCGGGGCCGGCAGCGTCATCTCGG
GCCGCGTGGCGTATGTGCTTGGGCTGCAGGGCCCAGCGATCACGGTGGACACGGCGTGCT
CGTCGTCGCTGGTGTCGCTGCACCTCGCCTGCATGGCCCTGCGCCGGGGCGAATGCGACC
TGGCGCTGGCCGGCGGGGTGATGGTGATGACCACGCCCGCGACCTTCGTGGAGTTCAGCC
GCCTCAAGGGCATTGCGCGAGATGGCCGCTGCAAGAGCTTCTCTGCCCAAGCGGACGGCG
TCATCTGGGCCGAGGGGTGCGGGATGCTGGTGCTGAAGCGGCTGTCGGACCCGCGGCGCG
ACGGTGACCGTGTGCTTGCGGTGGTCCGTGGGTCAGCGGTGAACCAGGACGGTCGCAGCC
AGGGCCTGACGGCGCCGAACGGCCCCGCGCAGCAGCGGGTGATCCAGCAGGCGCTGTCGT
CGTGCGGGCTGTCGCCCGAGGACATCGACGCGGTGGAGGCGCATGGTACGGGCACGAGCC
TCGGAGATCCCATCGAGGCGGGAGCGCTTGCGGAGGTGTTCGGGCCTGGGCGCAAGGCCG
AGCGGCCGCTGTACCTGGGCTCGTCGAAGTCGAACCTCGGGCACGCGCAGTCGGCCGCCG
GCGTGGCTGGGGTGATCAAGATGGTGCTGTCGATGCAGCACGAGGTGCTGCCGCGGACGC
TGCACGCGGAGCAGCCGAGCCCGCACATTGGGTGGGAGGGGAGCGGGCTGTCGTTGCTGC
AAGAGGCGCGTCCGTGGCGGCGCAACGGCCGGGCGCGGCGCGCGGGCGTGTCGTCGTTCG
GGATCAGCGGGACGAACGCCCATGTCATCCTCGAAGAGGCGCCGGTGGAGGCGGTGCGCG
AGCCGGTGGCGGCGGAGGCGGCAGAGGGTGCAGCGATGCCGCTGTTGCTGTCGGGGCGAG
ACCAGGCCTCGGTGGCGGCGCAGGCGGGGCGCTGGGCGAAGTGGCTGGAAGAGCACGCGG
AGGTGGGGTGGTCGGACGTGGTGAGGACGGCAGCGCTGCACCGGACCCACTTCACCACGC
GGGCGTCGGTGCTTGCGGCGAGCGCTGCGGAAGCTGTGGAGGGGCTGAGGGCGCTGTCGG
AGGGTCGAGCAGCGGCGGGGGTGGTGCGCGGGACGGGAGTGCGCGGGGGGAAGCTTGCGG
TGCTGTTCACGGGGCAGGGCAGCCAGCGGCTCGGGATGGGGAAGAGGCTTTACGAAGTGT
ACCCCGTGTTCCGTGCGGCGTTCGACGAGGTGTGCGAGGCGCTGGACGCGCACCTCGCGC
GTGGTCTGCGGGAGGTGGTGTTCGCGGCTGCGGGCAGCGAGGAAGGAGCGTTGCTGGAGC
GGACGGAGTACACGCAGCCCGGGCTGTTTGCGCTGGAAGTGGCGCTGTACCGTCAGTGGG
AGTCGTGGGGGCTGAAGCCCGCTGCGCTGCTGGGGCACTCGATAGGAGAGCTGAGCGCTG
CGCACGTGGCGGGTGTGCTGAGCCTTGCGGACGCAGCGAAGCTAGTGTGCGCCCGCGGTC
GGCTGATGCAOGGGTGCGAGGCCGGGGGAGCGATGGTGTCGGTGGAGGCCTCGGAGCCGG
AGGTGCAGCGGGCGCTGTCGGAGGTCGGGGCGCAGGGGCGACTGAGCATCGCCGGGCTGA
ACGCGCCGATGCAGACGGTGCTGAGCGGGGACGAAGCGGCGGTGCTCGCGGTGGCGCGAC
GGCTGGAGGCGCAGGGCCGGCGCACGCGGCGTCTGCGTGTGTCGCACGCGTTCCACAGCG
CGCACATGGACGGGATGCTGGAGGAGTTCGGGAAGGTGGCGCGGGAGTGCACGTACGCGC
GGCCGCAGCTGGCGGTGGTGAGCGGCGTGACGCGCGAGCTCGGTGGCGAAGAAGCGCTGA
TGTCGGCCGAGTACTGGGTGAGGCAGGTGCGCGAGGCGGTGCGCTTCCTGGACGGGATGC
GCACGCTTGCGGCGGCGGGGGTGAGCACATACGTCGAGTGTGGGCCGGATGGCGTGCTGT
GCGCGCTGGGGGCGGGGTGCCTGCCGGAGGGAGCCGAGGCGACGTTTGTGGCGAGCCTGC
GGCGAGAGCAGGAGGAAGAGCGCGCGCTGGCGACGGCGGTGGCGACAGTGCACGTGCAGG
GGCACGAGGTGGACTGGGCCCGGGTGCTATCCGGTCAGGGCGGTCGTGCCGTGGAGCTGC
CGACGTACGCATTCCAGCGGCAGCGCTACTGGCTGGAGGCGCCGAGGGCGCGCGGTGACG
TGGGCTCGGCGGGGCTGAAGGCGGCCGCCCATCCGCTGCTCGGCGCCGCGACGAAGCTCG
CCGACGGCGAGGGGCACCTGTTCACAGGGAGGCTGTCGCTGGCGGAGCATGCGTGGCTTC
GGGATCATGGTGTGTTTGGCCAGGTGGTGTTTCCGGGCACGGGCATGCTGGAGGTTGCGC
TGGCGGCTGGGCGCGCGGTGGGCAGCCGGTCGCTGTCGGAGCTCACGCTCGCCGAGCCGC
TGGTGCTGGCCGAGGAGGGCGCGGCGCGGCTGCAGGTGATGATCGGAGCGCCAGATGCGG
CGGGCCGGCGCGAGGTCGGGCTGTACAGCCAGTCTGAGCAGGCCCCGGAGGACGCGCCGT
GGGTGCAGCACGCGACGGGGGTGTTGACGGACGAGCCCCCCGGCGCTCCTGGCGAGCTCG
ACGAGCTGTCGACGTGGCCTGTGCCGGGCGCGGAGGAGGTGGACCTGTCCGGGGTTTACG
GGCGGCTGCGTGAGCGCGGGCTCGACTACGGTCCGACGTTCCAGGGCCTCGTGGAGCTCT
GGCGTCGAGGCACGAGGCTTTACGGCCGGGTGGTGTTGCCCGAGAGCGCGATGGACAGCG
CCGAGGCGTATGGGGTGCACCCGGCGCTGATGGATGCCGCGCTGCATGCGCTGTTCGCGG
CCTCTTCGGAGGCGGCTGGAGCGGACATGGCGTTGCTGCCCTTCTCGTGGTCGGACGTGG
CGCTGCTCGCGACGGGACCGCGTGAGCTCCGGGTGATGCTTGAGCTTGAAGGCGACATGA
CGCAAGCGGTTGCGTCGCTGTGGGTCGCGGACGCGGCGGGCCAGGCTGTGGCCAGCGTCG
GCGCCCTGCATCTGCGGCGAGCGACAGCGGAGCAGCTGGCAGTGGCCAGCCGTGCCAATG
CCCAGCAGCTGTACCGGGTGGACTTCCAACCGGTGAGCGTGGTGGCGAGCGTCCCGGAGG
CGGGCTCGCTGGTGGTCGTCGGTGCAGCGGAGGGACGAGGGAGGCTGGCCGAAGCCCTGG
GGGCGGAGGCGATTGCCGATCTCGATGCATTGGTTGCGCGCCTCGAGCATGGCGCGAGCG
CGCCTGAGCGGGTGGTGGTCGACGTCACCGCCGCGAGCCCGAGCCCGTTGGACGTGGCGG
TGTCGTCGCATGAGGCGACGAGGCAGGCGCTGTCGCTGCTGCAAGCGTGGCTGTCGGAGC
CGCGGCTCGACGATGTCGAGCTTGTGTGGATGACGCGAGGCGCGGTGGGCGCGGCGCCGA
ACGACGCCGTCGAGGACCTGGCGCACGCGCCGCTGTGGGGGCTTATTCGTACGGCGCGAA
GCGAGCACCCCGAGCGCCGGCTGCGCTTGATCGATGTGGGGACCGAGCCCGTGGACGCTG
GGCTGCTGGCGCGGGCGCTGGCGACGGCGGCGGAGCCGGAGCTCGCGCTGCGCGGGGGCG
CTGCGCTGGCCGCGCGCCTGGTGCGCGTACAGGCGGCAGCGGAAGAGCTCACCCGAGGAG
CCCGCGAGCTGGACCCTGCGGGCACGGTGCTGGTGACCGGCGGGACAGGGGAGCTGGGGC
AGGCGGTCGCGGCGCACCTGGTGCGCGCGCACGGCGTGAGGCACCTGGTGCTGACGTCGC
GGCGCGGGCTGGAGGCGCCCGGGGCCCGCGAGCTTGTGCAATCGCTCGCGGAGCTCGGCG
CCGAGACGGTGACGGTGGCTGCGTGCGACGTGTCGAAGCGGGAGGAGGTCGCGCGCGTGC
TGGCCGGCATCGACGCGGCGCGCCCGCTGAGCGCGGTGCTGCACCTGGCCGGCGTGCTCG
ACGACGGCGTCCTGTCGTCGCAGACGCCGGAGCGCATTTCACGGGTGTTCGCGCCGAAGG
TGGATGGGGCGCTGCACCTGCACGAGCTGACGCGGGAGCTGGATCTCGCGGCGTTCGTGC
TGTTCTCGTCGGTGGCCGGCACGTTTGGCACGGCGGGCCAGAGCAACTACGCGGCGGCGA
ACACGTTCCTCGACGCGCTCGCGGCGCACCGGCGCGGCGGCGGGCTCGCGGCGACGAGCC
TGGCGTGGGGCTTCTGGACACAGGCGGGCGTGGGGATGACAGCGCACCTGGGCAAGGCCG
AGCTGTCGCGCATGAGGCGCAACGGGTTCGTGCCGATGCCGGTGGAAGAGGGCCTCGCTC
TGCTGGACGCCGCGCTTTTGCGCTCCGAAGCGAGCCTGGTCCCAGTGCACCTGGATCTCG
CGCAGCTGCAGCGTGGGCTGGAGTCCAGCGGCGAGCTGCCGGCGCTGTTTCGTGCGCTGT
TGCGCCCCAGCTTGCGCAAGGCGTCCACCGCAACGGCAACGAGGCGAGACGCCTCGGCGC
TGCGCGGGCGCCTCTCGGCGCTGCCGGAGGCGGAGCGGCTGAATGCGCTCATCGAGCTGG
TGCGGGGCGAGGTCGCCGCGGTGCTCGGGCTCCAGCGCAGCGAGGCCGTGGGGGCAGAGC
AGGTGCTGAAGGGCCTCGGGCTCGACTCGCTGATGGCGGTGGAGCTGCGCAACCGCCTCG
CCGCCCGGGCGGAGACGTCCTTGCCGGCGACGCTGGTCTTCGACTACCCGACACCGCGAG
CGATCGCAGAGCTGCTGCTGAAGCAGGCGTTCTCGGGGCTGCAGGTGAAGGAAGCGCGGG
CGCGTCGTCATGCGGGGAAAGACGAGGCGGTGGCGATCGTATCGATGGCGTGCCGGCTGC
CGGGAGGCGTCGAGACGCCGGAAGACTACTGGCGTCTCTTGGCCGAGGGGAAAGACGCGA
TCGACGGCCTCCCTGCGCGCTGGGAGGCGCTTTCGGTCTACGACCCCGATCCGGAGGCGG
TGGGCAAGAGCTACGCGCGCGAGGGTGGATTCTTGCGGGACATCGACCTGTTCGACGCGG
ACTTCTTCGGGATATCGCCGCGCGAGGCGCAGTCGATGGATCCCCAGCAGCGGCTGGTGC
TGGAGACGGCGTGCGAGGCATTGGAGCGTGCCGGCGTGCGCCCGTCGGCGCTGAGCGGGA
GCGCCACCGGAGTGTATCTCGGGTCGATGGGCTCGGACTACGGTGCTCTTCATACCGGCG
GGCTGGAAGCGCTGGACGGGTACCGGGGCACCGGGAGCGCGGCGAGCGTGCTCTCAGGCC
GTGTGGCCTACGTGCTCCGGTTGCAGGGCCCAGCGATCACGGTGGACACGGCGTGCTCGT
CGTCGCTGGTGTCGCTGCACCTCGCGTGCACGGCGTTGCGTCAGGGTGAATGCGACCTGG
CGCTGGCCGGCGGGGTGACGGTGATGAGCACCCCCGCGTTGTTCGTGGAGTTCAGCCGGC
TCAAGGGGATGGCCCGCGACGGCCGCTGCAAGAGCTTCTCTGCGCGAGCTGACGGCGTCA
CCTGGTCCGAGGGGTGCGGGATGCTGGTGCTGAAGCGGCTGTCGGACGCGCGGCGCGACG
GTGACCGTGTGCTTGCGGTGGTCCGCGGGTCAGCGGTGAACCAGGACGGTCGCAGCCAGG
GCCTGACGGCGCCGAACGGCCCCGCGCAGCAGCGGGTGGTCCAGCGGGCGCTCTCGTCGT
GCGGGCTGTCGCCCGAGGACATCGACGCGGTGGAGGCGCACGGGACAGGCACGAGCCTCG
GAGATCCGATCGAGGCGGGAGCGCTCGCGGAGGTGTTCGGGCCTGGGCGCAAGGCCGAGC
GACCGCTGTACCTGGGCTCGTCGAAGTCCAACCTGGGGCATACGGGGCCTGCGGCGGGTG
TAGTCGGTGTGCTCAAGATGGTGCTGTCGATGCAGCACGAGGTGCTGCCGCGGACGCTGC
ACGCGGAGCAGCCGAGCCCGCACATTGGGTGGGAGGGGAGCGGGCTGTCGTTGCTGCAAG
AGGCGCGTCCGTGGCGGCGCAACGGCCGGGCGCGGCGCGCGGGCGTGTCGTCGTTCGGGA
TCAGCGGGACGAACGCGCATGTCATCCTCGAAGAGGCGCCGGTGGAGGCGGCGCGCGAGC
CGGTGGAGGCGGTGCGCGAGCCGTTGGCGACGGAGGGTGTTGCGATGCCGCTGTTGCTGT
CGGGGCGAGACGAGGCCTCGGTGGCGGCGCAGGCGGAGCGCTGGGCGAAGTGGCTGGAAG
AGCACGCGGAGGTGGGGTGGTCGGACGTGGTGAGGACAGCGGCGCTGCACCGGACGCACT
TCGCCTCACGCGCATCGGTGCTTGCGGCGAGCGTGTCGGAGGCGGAGGAGGCGCTGCGGG
CGCTGTCGCAGGGTCGCGGCCACCGGGCGGTGTCGGCGGGCACGGCGCGTGCGCGAGGCA
AGGTGGTGTTCGTGTTCCCCGGCCAAGGGAGCCAGTGGCCGGGGATGGGCCGGGCGCTGC
TGGAGCAGAGCGCGGCGTTCGCGGAGGCGGTGCAGGCGTGCGATGAGGCGCTGCGGCCGT
GGACGGGCTGGTCTGTGCTGTCGGTGCTGCGCGGCGAGGCGGGTGAGGCAGGTGAGGAGC
AGCCGTCGCTGGAGCGGGTGGACGTGGTGCAGCCCGCGCTGTTCGCGATGTGCGTGGGTC
TGGCCGCGGCGTGGAGGTCGCTGGGGCTGGAGCCTGCGGCGGTGGTGGGCCACAGCCAGG
GCGAGGTGTCGGCGGCGGTGGTGTGCGGGGCGCTGTCGCTTGCGGAGGGAGCGCGGGTAG
TGGCGCTGCGCAGCCAGGCGGTGCGGCAGCGGTCGGGGATGGGGGCGATGATGCTGGTCG
AGCGGCCGGTGTCGGAGGTGCAGGAGCGCATCGCGCCGTACGGGGAGGCGCTTGCGATAG
CGGCGGTGAACACGTCGAGCTCGACGGTGGTGTCGGGTGACGTGGAGGCGGTGGACGGGC
TGATGGGGGAGCTGACGGCAGAAGGTGTGTTCTGCCGGAAGGTGAACGTCGACTACGCGT
CGCACAGCGCGCACATGGATGCGCTGCTGCCCGAGCTAGGAGCGAAGCTGTCGTCGCTCA
GGCCGAAGGCGACGCAGCTGCCGTTTTACTCGACGGTGGCAGGAGAGGTGTCGCGAGGCG
AGGCGCTGGACGGCGAGTACTGGTGCCGCAACCTTCGGCAGACGGTGCGCCTGGACCGAG
CGCTGTCGAAGCTGCTGGAGGACGGGCACGGTGTGTTCGTGGAGGTGAGCGCGCACCCGG
TGCTGGCGATGCCGCTGACGACGGCGTGCGGGGAGGCGCAGGGGGTGGTGGTGGGGAGCC
TGCAGCGCCACGAAGGTGGGTTGTCGCAGCTGTACAGGACGCTGGGGCAGCTGCACGTGC
AGGGGCACGAGGTGGACTGGGCACGGGTGCTGCCGGGCCATGGCGGTCGTGCCGTGGAGC
TGCCGACGTACGCATTCCAGCGGCAGCGCTACTGGCTGGAGGCGCCGAGGGCGCGCGGTG
ACGTGAGCTCGGCGGGGCTGAAGGCGGCCGCCCATCCGCTGCTCGGCGCCGCGACGAAGC
TCGCCGACGGCGAGGGGCACCTGTTCACAGGGAGGCTGTCGCTGGCGGAGCATGCGTGGC
TTCGGGATCATGGTGTGTTTGGCCAGGTGGTGTTTCCGGGCACGGGCATGCTGGAGGTTG
CGCTGGCGGCTGGGCGCGCGGTGGGCAGCCGGTCGCTGTCGGAGCTCACGCTCGCCGAGC
CGCTGGTGCTGGCCGAGGACGGCGCGGCGCCGCTGCAGGTGATGATCGGAGCGCCAGATG
CGGCGGGCCGGCGCGAGGTGGGGCTGTACAGCCAGCCTGAGCATGCCCCGGAGGACGCGC
CGTGGGTGCAGCACGCGACGGGAGTGTTGACGGACGAGCTCCCCGGCATCCCTGACGAGC
TCGACGAGCTGTCGATGTGGCCTGTGCCGGGCGCGGAGGAGGTGGACCTGTCCGGGTTTT
ACGAGCGGCTGCGTGAGCGCGGGCTCCACTACGGTCCGACGTTCCAGGGCCTCGTGGAGC
TGTCGCGCCAAGGCACCAGGCTTTATGGCCGGGTGGTGCTGCCAGGAACCGAGAAGGACA
GGGCGGAGGCGTATGGCTTGCATCCCGTCCTGATGGATGCAGCACTGCAGGTGCTTGGCG
CAGCCGGCGAGGGGCATTGGGAGGCGGATGCGTTGTTTATGCCCTTCTCCTGGGCAGACG
CCGCAACGCATGCCACGGGTCCGAGCGAGCTTCGGGTGCGCGTGGAGCTCGAAGAGACAG
ACGGCTCCACGCAGGCAACGGCATCTCTGTGCGCTGCAGATGCCGCAGGCCAGCCGGTGG
CGAGCGTCGGTGCTTTGCGTTTGCGCCGTGTGACGGCCGAGCAATTGAGGGCGGTCACCC
GCACCGATGAGCAGCACCTGTACCGGGTGAGCTTCCAGCCCGTGAGCCTCGCGCAAGCCC
CCCTGGAGGCGGGCTCGCTGGTGGTCCTCGGTGCAGCGGAGGGACGAGGGCAGCTGGCCG
ACACCCTGGGGGCGGAGGCGATTGCCGATCTCGATGCATTGCGCGCTTGGATCGAGCGGG
GCGCGCCAACGCCTGTGCGGGTGGTGATCGACACGAACGCTGCCAGCTCACCGCGCTCGG
ACGTGGCGGGGTCGTCGCACGAGGCGACGAGGCAGGCGCTGTCGCTGCTGCAAGCGTGGT
TGTCGGAGCCGCGGCTCGACGCTGTCGAGCTGGTGTGGGTGACCCGGGGCGCGGTCAGCG
CAGCTCCGGACGACGCCGTCGAGGACCTGGCGCACGGGCCGCTGTGGGGGCTTATTCGCA
CGGCGCGCAGCGAGCACCCCGAGCGCCGGCTGCGCTTGATCGATGTGGGGACCGAGCCCG
TGGACGCTGGGCTGCTGGCGCGGGCGCTGGCGACGGCGGCGGAGCCGGAGCTTGCGCTGC
GCGGGGGCGCGGTGCTGGCCGCGCGGCTGGTGCGCGTACAGGCGGCAGCGGAAGAGCTCA
CCCGAGCCCGCGGGCTGGACCCTGCGGGCACCGTGCTGGTGACCGGAGCCGTGGGCGGTC
TGGGGCAGGCGGTGACACGCCATCTGGTGCGCGCGCACGGGGTGAGGCACCTTGTGCTGA
CGTCGCGCCGGGGGCTGGAGGCGCCCGGGGCCCGCGAGCTTGTGCAATCGCTCGAGGAGC
TCGGCGCCGAGACCGTGTCGATGGTGGCGTGCGACGTGTCGAAGCGGGAGGAGATCGCGC
GCGTGCTGGCCGGCATCGACGCGGCGCGCCCGCTGAGCGCGGTGCTGCACCTGGCTGGCG
TGGTCCATGATGGCGTGATTCAGACGCAGACGGCCGAGCGCCTCGCGTGGGTGCTGGCGC
CGAAGGTGGACGGGGCGCTGCACCTGCACGAGCTGACGCGGGAGCTGGATCTCGCGGCGT
TCGTGCTGTTCTCGTCGGCGGCCGGTACGTTGGGCATGGCGGGCCAGGGCAATTACGCGG
CGGCGAATACGTTCCTCGACGCGTTCGCGGCGCACCGCCGCGGCCGCGGGCTCGCGGCGA
CGAGCCTCGCCTGGGGTGTCTGGACACCGGCCGGTGGTGGCATGGCGGCACAGCTGGGGG
CCGCGGAGCTGGCACGGTTCAGTCGCTACGGAGTCGTGTCGATGTCCGTGGAAGAGGGGC
TTTCGCTGCTGGACGCCGCGCTGTCACGCCCTGAAGCGAGCCTGGTCCCTATGCACCTGG
ATCTCGCGCAGCTGCAGCGTGGGCTGGAGGCCAACGGCGAGCTGCCGGCGCTGTTTCGTG
CGCTGTTGCGCCCCAGCTTGCGCAAGCCGTCCACGGCGACGAGGCGAGACGCCTCGGCGC
TGCGCGGGCGCCTCTCGGCGCTGCCGGAGGCGGAGCGGCTGAATGCGCTCATCGAGCTGG
TGCGGGGCGAGGTCGCCGCGGTGCTCGGGCTCCAGCGCAGCGAGGCCGTGGGGGCGGAGC
AGGTGCTGAAGGGCCTCGGGCTCGACTCGCTGATGGCGGTGGAGCTGCGCAACCGCCTCG
CCGCCCGGACGGAGAOGTCCTTGCCGGCGACGCTGGTCTTCGACTACCCGACGCCGCGGG
CCATCGCGGAGTTGCTCCTGAAGCTGGCCTTTTCGGGACCGCAGGTGATGGGAGCCCGCA
GGGGGGTGCGTCGTCATGCGGGGAAAGACGAGGCGGTGGCGATCGTATCGATGGCGTGCC
GGCTGCCGGGAGGCGTCGAGACGCCGGAAGACTACTGGCGTCTCTTGGCCGAGGGGAAAG
ACGTGATCGAGGGCCTCCCTGCGCGCTGGGAGACGCTTTCGGTCTACGACCCCGACCCGG
AGGCGGTGGGCAAGAGCTACGCGCGCGAGGGTGGATTCTTGCGGGACATCGACCTGTTCG
ACGCGGACTTCTTCGGGATATCGCCCCGAGAGGCGCAGGCGATGGATCCCCAGCAGCGGC
TGGTGCTGGAGACGGCATGGGAGGCATTGGAGCGTGCCGGCGTGCGCCCGTCGGCGCTGA
GCGGGAGCGCCACCGGGGTGTATCTGGGGGCCGCGGGTTCGGACTATGGCGCTTACCAGG
GTGGCGGGCTGGAGATGCTGGACGGGTACCGGGGCATCGGGACCGCGGCGAGCGTGCTCT
CAGGCCGTGTGGCCTACGTGCTCGGGCTGCATGGCCCAGCGATGACGGTGGACACGGCGT
GCTCGTCGTCGCTGGTGTCGCTGCACCTCGCGTGCACGGCGTTGCGTCAGGGTGAATGCG
ACCTGGCGCTGGCCGGCGGGGTGACGGTGATGAGCACCCCCGCGTTGTTCGTGGAGTTCA
GCCGGCTCAAGGGGATGGCCCGAGACGGCCGCTGCAAGAGCTTCTCTGGGCAGGCGGACG
GCGCGGGCTGGTCGGAGGGGTGCGGGATGCTGGTGCTGAAGCGGCTGTCGGACGCGCGGC
GCGACGGTGACCGTGTGCTTGCGGTGGTCCGTGGGTCAGCGGTGAACCAGGACGGTCGCA
GCCAAGGCCTGACGGCGCCGAACGGCCCCGCGCAGCAGCGGGTGATCCAGCAGGCGCTGT
CGTCGTGCGGGCTGTCGCCCGAGGACATCGACGCGGTGGAGGCGCATGGTACGGGCACGA
GCCTCGGAGATCCGATCGAGGCCGGAGCGCTCGCGGAAGTGTTCGGGCCTGGGCGCAAGG
CCGAGCGACCGCTGTACCTTGGCTCGTCGAAGTCGAACCTTGGGCACGCGCAGGCTGCGG
CGGGCGTAGCCGGTGTGCTCAAGATGGTGCTGTCGATGCAGCACGAGGTGCTGCCGAAGA
CGCTGCACGCGGAGCAGCCGAGCCCGCACATTGGGTGGGAGGGGACCGGGCTGTCGTTGC
TGCAAGAGGCGCGTCCGTGGCGGCGCAACGGCCGGGCGCGGCGCGCGGGCGTGTCGTCGT
TCGGGATCAGCGGGACGAACGCCCATGTCATCCTCGAAGAGGCGCCGGTGGAGGCGGCTC
GCGAGCCGGTGGAGGCGGTGCGCGAGCCGGTGGAGGCGGAGGGTGTTGCGATACCGCTGT
TGCTGTCGGGGCGAQACGAGGCCTCGGTGGCGGCGCAGGCGGGGCGGTGGGCGAAGTGGC
TGGAAGAGCACGGGGAGGTGGGGTGGTCGGACGTGGTGAGGACGGCGGCGCTGCACCGGA
CGCACTTCGAGTCGCGGGCGTCGGTGCTTGCGGCGAGCGCTGCGGGAGCTGTGGAGGGTC
TTCGCGCGCTGTCGTCGGGGCGGCCGGATGCGGCGGTGGTGAGCGGGACGGCGAAGCGAG
GCGGGAAGCTTGCGGTGCTGTTCACGGGGCAGGGCAGCCAGCGGCTCGGGATGGGGAAGA
GGCTTTACGAAGTGTACCCCGTGTTCCGTGCGGCGTTCGACGAGGTGTGCGAGGCGCTGG
ACGCGCATCTCGACCGTGGGTTGAGAGAGGTGGTGTTCGCGGCCGCGGGCAGCGAGGAAG
GAGCGTTGCTGGAGCGGACGGAGTACACGCAGCCCGGGCTGTTTGCGCTGGAAGTGGCGC
TGTACCGTCAGTGGGAGTCGTGGGGGCTGAAGCCCGCTGCGCTGCTGGGGCACTCGATAG
GAGAGCTGAGCGCTGCGCACGTGGCGGGTGTGCTGAGCCTTGCGGACGCAGCGAAGCTAG
TGTGCGCCCGCGGTCGGCTGATGCAGGGGTGCGAGGCCGGGGGAGCGATGGTGTCGGTGG
AGGCCTCGGAGCCGGATGTGCAGCGGGCGCTGTCGGAGGTCGGGGCGCAGGGGCGACTGA
GCATCGCCGGGCTGAACGCGCCGATGCAGACGGTGCTGAGCGGGGACGAAGCGGCGGTGC
TCGCGGTGGCGCGACGGCTGGAGGCGCAGGGCCGGCGCACGCGGCGTCTGCGTGTGTCGC
ACGCGTTCCACAGCGCGCACATGGACGGGATGCTGGAGGAGTTCGGGAAGGTGGCGCGGG
GGTGCACGTACGCGCGGCCACGGCTGGCGGTGGTGAGCGGCGTGACGGGCGAGCTCGGTG
GCGAAGAAGCGCTGATGTCGGCCGAGTACTGGGTGAGGCAGGTGCGCGAGGCGGTGCGCT
TCCTGGACGGGATGCGCACGCTTGCGGCGGCGGGGGTGAGCACATACGTCGAGTGTGGGC
CGGATGGCGTGCTGTGCGCGCTGGGGGCGGGGTGCCTGCCGGAGGGAGCCGAGGCGACGT
TTGTGACGAGCCTGCGGCGAGAGCAGGAGGAAGAGCGCGCGCTTGCGACGGCGGTGGCGA
CAGTGCACGTGCAGGGGCACGAGGTGGACTGGGCCCAGGTGCTGTCGGGCCGTGGCGGCC
GGCCCGTGGAGCTGCCGACGTACGCGTTCCAGCGGCAGCGCTACTGGCTGGAGGCGCCCA
AGACGACGGCGGCACAGGCGAATGTCTCGTGGCCGGAGCGTGCGTTGTGGGACGCGGTGC
AGAAAGGCGAAGGCGTTGCGGATCTGCTGGAGCTGCCTGACGACGTGCGCGAGAGCGTCG
CGCCGCTGCTGCCGTACCTTGCGGCGTGGCGCCGGAGAACGGACGCAGAAGCCACGGTGT
CTGGCTGGTTGTACGAGGAGGCGTGGCAAAGGGAAGCCAGCGCTGCCAGGGGCAAGCCGG
ACGTGAGGGGCAGATGGCTGCTGGTGTCATCTCCGCGTGCCGGAGGGCTGACCGCGGCGG
TGAGTGATGCGCTCGCAGCTGCGGGTGCAGAGGTGATCATCGAGCCGGCGACCGAAGAGC
GAGCGCAGCTGGCGGCGAGGTTGAGAGGGCTGGAGGGCGAGCTGCGTGGCGTCGTGGCGC
TGAGCGCGCCTGGGGAGCAAGGTGCGCTGGAGGAAGGGCGAGGGCCTCGCGGAGTGTACG
AGGTGCTGGCGCTGGCGCAGGCGCTCGGTGACGCTGGGCTCGATGCGCGGCTCTGGGTGT
TGACGCAGGGAGCGGTGAGCACGCAGGCAAGCGAAGGGGTGTCCGACCCTGCGCAGGCGC
TGACGTGGGGGTTGGGGCGGGTGGTGGGGCTGGAGCACCCCGAGCGCTGGGGTGGACTGG
TAGACCTGCCGGCGGAGGTGGACGCGGAAGCGGTGCAGCAGGTGCTGAGGACGCTCGTTG
CCGAGGACCACGAGGACCAGGTGGCGGTGCGACGCGGTGGGCGTCTTGTGCGGCGCATCG
TGCGGGTGAGTGGAGAGGACGGCGGAGCGGGGTGGAAGCCGCGTGGCACGGTGCTCATCA
CGGGTGGAGTGGGAGGGCTCGGGAGCCATCTGGCTCGCTGGTTGGCGGAGCGGGGAGCAG
AGCACCTGGTGCTGGCGTCACGCCGGGGCGCCGCGGCAGCGGGCGCGCGCGAGCTTCGGG
AGGAGCTTGAGGGGCGGGGCGCGCGCGTGACGCTTGCGGCGTGCGATGTGTCGGAGCGAG
CGCAGGTCGAGGCGCTGGTGAGGGAGCTTGAGCAGGACGAAGCGCCGCTGAGCGCGGTGG
CGCATCTGGCGGGGATAGTCCGCCGCGTGCCGGTGCGAGAGCTCGCGCCCGAGATGCTGG
CGCAGGAGCTCGCGGCGAAGGTCAACGGAGCATGGCACCTGCAGGAGCTGCTGGCAGAGC
GCGAGCTGGATGCGTTCGTGCTTTATGGCAGCATCGCTGGGCTGTGGGGCTCTGGGACGC
AGGCCGGGTACGGCGCGGCGAACGCAGGGCTCGACGCCCTCGCGCGCTACCGGCGTGCCC
GAGGGCAGACGGCGACGGTGCTGCACTGGGGCCCGTGGTCCGGAGGCGGGATGGTGAGCG
ACGAGGCCGAGCCGCAGCTCCGGAGCCGCGGGCTGGTGCCGATGTCGCCGGACAAGGCGC
TTTGCGGGCTCGAGGTTGGGCTGCGGCGCACGTCGGTGGCGATCGCGGACGTGGACTGGT
CGCGCTTCGCGCCGCTGTTCTGCGCGGCGCGGCCGAGGCCGCTGCTGTACGGGATCGAGC
AAGCGCGCCATGCGCTGGAGGGCCGGACACCGCAGCAGGCCGCGGGCGGAGCGGGGGACA
AGGCGCTGCGGGAGATGCTGCTCGGCCTGCCGGCTGTGGAGCGGAGCGAGCGGCTGCGCG
AGCTCGTGGCGAGCGAGACGGCGGCGGTGCTGGGCGTGAAGGATCCGAGCGGGCTGGACC
CGGAGCGAGGCTTTCTGGACCTCGGGCTGGACTCGTTGATGGCGGTGGAGCTGTCGAAGC
GGCTGCAGCAGCGGACGGGGGTGTCGGTCACAAGGACGTTGATCTTCGATTATCCGACGC
AAGGCGAGGTAACGCGCTGGCTGCTGGAGCAGCTGATGCCGCCGGAGCGACCGGCGGCGG
ACGAGCACGGCGTGAGCCGTGGACCGGAGCGGAGCGCGCCGATAGCGATCGTGGGCGTGG
GGCTGCGCATGCCGGGCGGAGCGAACGATCTGGAGAGCTTCTGGCAAGTGCTCGTGGAGG
GGCGGGATACGCTAAGGCCGATCCCGACCGACCGTTTCGACGTGGAGGCGATGTACGATC
CTAACCCCGAGGCCAAGGGCAAGACGTACGTGAAGCATGCCTCGCTGCTGGACGACGTGG
CATCGTTTGACGCGGGGTTCTTCGGGATAAGCCCGCGCGAGGCGGAGCCGATGGATCCGC
AGCACCGGCTGCTGCTGGAGACTGCGTGGAGCGCCCTGGAGGACGCGGGAGTGCGTCCAG
ACCAGCTGAAGGGCTCGGACACGGGTGTGTTCGTGGGCGTGGCGCCGAGCGAGTATGCGA
GCTATCGCGGCAAGAGCGCGAACGAAGATGCGTATGCGCTGACGGGGACCGCGCTGAGCT
TCGCAGCGGGCCGTGTGGCATATCATCTCGGGCTGCAAGGCCCTGCGGTGTCGGTCGATA
CGGCGTGCAGCTCGTCGCTGGTGGCGCTGCACCTGGCGTGCGACGCATTGAGGCGCGGCG
ATTGCGAGGTGGCTCTGGCGGCCGGCGTGCAGGTGCTCGCGAACCCGGCGGGGTTTGTGC
TGCTGTCGCGCACGCGTGCGGTCTCGCCCGACGGACGGTGCAAGACGTTCTCCCAGGCGG
CCGACGGCTACGGCCGCGGCGAGGGCGTCGGGGTGGTGGTGCTGATGCGTCTTTCGGACG
CACAGGCGCAGGGGATGCGGGTGCTGGGCGTGGTGCGGGGCACGGCGGTCAATCAGGACG
GCGCGAGCAGCGGGATCACGGCGCCGAACGGCACGGCCCAGCAGAAGGTGGTGCGCGCGG
CGCTGCGGAACGCGGGGCTGGAGGCGTCGAGCATCGATGTGGTCGAGTGCCACGGTACGG
GCACGTCGCTGGGCGATCCGATCGAGGTGCAAGCGCTCGGCGCGGTGTACGGGCAAGGCA
GGGAGGCGACTCGCCCGCTGCGGCTTGGAGCGGTCAAGAGCAACATCGGTCACCTGGAGT
CGGCCGCCGGCATCGCCGGAGTGTGCAAGATCCTGGCGGCGTTTCGGCATGAGGCCCTGC
CGGCGACGTTGCACAGCTCGCCGCGCAACCCCCAGATCTCCTGGGAGAGTCTGCCGGTGC
AGGTGGTCGACCGCCTGACCGGCTGGCCTCGGCGCGCCGACGGCCTCCCCCGCTTTGCGG
GCGTGTCGTCGTTTGGCATCAGCGGGACGAACGCCCATGTCATCCTCCAAGAGGCGCCGC
TTGAGGCGGTGCGCGAGCCGGCGGCGGTGCGCGAGCCGTTGGCGGCGGAGGGTGTCGCGA
TCCCGCTGTTGCTGTCGGGGCGAGACGAGGCCTCGGTGGGGGCGCAGGCGGAGCGCTGGG
CGAAGTGGCTCGGAGAGCACGCGGAGGTGCGGTGGCCGGACGTGGTGAGAACGGCGGCGC
TGCACCGGACGCACTTCGCCTGGCGCGCATCGGTGCAGGCGGCGAGCGTGTCGGAGGCGG
TGGAGGGGCTGAGGGCGCTGTCGGAGGGTCGAGCCGCGGCAGGTGTGGTGCGCGGGACGG
GAGGGCGCGGGGGGAAGCTTGCGGTGCTGTTCACGGGGCAGGGGAGCCAGCGGCTCGGGA
TGGGGAAGAGACTTTACGAAGTGTACCCCGTGTTCCGTGCGGCGTTCGACGAGGTGTGCG
AGGCGCTGGACGCGCATCTCGACCGTGGGTTGAGAGAGGTGGTGTTCGCGGAAGCGGGCA
GCGAGCAGGAGGCGCTGCTGGAGCGGACGGAGTACACGCAGCCCGGGTTGTTTGCGCTGG
AAGTGGCGCTGTACCGGCAGTGGGAGGCGTGGGGAGTGAGGCCCGCGGCGCTGCTGGGGC
ACTCGATAGGAGAGCTGAGCGCTGCGCACGTGGCGGGCGTGCTGAGCCTTGCGGACGCAG
CGAAGCTAGTGTGCGCCCGCGGTCGGCTGATGCAGAGGTGCCAGGCGGGCGGAGCGATGA
TGTCGGTGGAAGCGTCGGAGCCGGAGGTGCAGGGGGCGCTGTCGGCGATGGGGCTGGAGG
GCCGGCTTGGGATCGCGGGCATCAACGGTCCGAGCCAGACGGTGCTGAGCGGGGACGAAG
CGGCGGTGCTGGAGGTGGGCAGGCGGTTCGAGGCGCAGGGCCGGCGCACGCGGCGTCTGC
GCGTGTCGCACGCATTCCACAGCGCGCACATGGACGGGATGCTGGAAGAGTACGGGAGGG
TGGCGCGGGAGTGCGCGTATGGGAGGCCGCAGGTACCCGTGGTGAGCGGCGTGACGGGCG
AGCTCGGTGGCGAAGAATCGCTGATGTCGGCCGAGTACTGGGTGAGGCAGGTGCGCGAGG
CGGTGCGCTTCCTGGACGGGATGCGCACGCTTGCGGCGGCGGGGGTGAGCACATACGTCG
AGTGCGGTCCGGATGGCGTGCTGTGCGCGCTGGGGGCGGGGTGCCTGCCGGAGGGAGCCG
AGGCGACGTTTGTGGCGAGCCTGCGGCGAGAGCAGGAGGAAGAGCGCGCGCTTGTGACGG
CGGTGGCGACGGTGCACGTGCAAGGGCACGAGGTGGACTGGGCCCAGGTGCTGTCGGGCC
ATGGCGGCCGGCCCGTGGAGCTGCCGACGTACGCGTTCCAGCGGCAGCGCTACTGGCTGG
AGGCGCCGAGGGCGCGCGGCGACGTGGGCTCGGCGGGGCTGAAGGCGGCCGCCCATCCGC
TGCTCGGCGCCGCGACGAAGCTCGCCGACGGCGAGGGGCACCTGTTCACAGGGAGGCTGT
CGCTGGCGGAGCATGCGTGGCTTCGGGATCATCAGGTGTTTGGCAAGGTGGTGTTTCCGG
GCACGGGGATGCTGGAGCTGGCGCTGGCGGCGGGGCGCGCGGTGGGCAGCCGGACGCTGT
CGGAGCTGGTTCTGGCCGAGCCGCTGGTGCTGGCCGAGGAGGCCGCGGCGCGGCTGCAGC
TGTCGGTCGGAGCGCCGGACGCGGCGGGCCGGCGCGAGGTAGGGCTGTACAGCCAGTCCG
AGCAGGCGCCGGAGGACGCGCCGTGGGTGCAGCACGCCACGGGCGTGTTGACGGACGAGA
TCCCCGGCGCCCCCGGCGAGCTCGACGAGCTGTCGACGTGGCCTGTGCCGGGCGCGGAGG
AGGTGGACCTGTCCGGGTTTTACGAGCGGCTGCGTGAGGGCGGGCTCGACTACGGTCCGG
TGTTCCAGGGCCTCGTGGAGCTCTGGCGTCGAGGCGCGAGGCTTTACGGCCGGGTGGTGT
TGCCCGGGAGCGCGAGGGGCAGCGCCGAGGCGTATGGGGTGCATCCGGCGCTGATGGACG
CCGCGCTCCACACGATGGTCGCAGCTTTCTCTCAGATGTCAGGGCCAGACGGCGTGTTGT
TGCCGTTCGCCTGGTCGGACGTGGCGCCGCACGCGACGGGGGCGAGCGAGCTTCGGATCC
GAGTGGAGATGCAGGAACAAAGCGCACAGCAGCCAGCGGCTTCGCTGTACGTCGCAGACT
GCACGGGGCAGGTCGTGGCGAGCATCGGCGCTCTACGTCTGCGCCGGGCGACGGCCGAGC
AGCTGCGGACCGCCGTTCACGCTGGTGGCCAACATATGTATCAGGTGAGCTTCCAGCCTG
TGGACCTCGCAGCACCTCCCTTGGTGACGGGCTCGCTGGTGGTCATCGGTGCACCGAAGG
GAGGAGCGCGGCTGGCCGAAGCCCTGGGGGCGGAGGCGATTGCCGATCTCGATGCATTGG
TTGTGCGCCTCGAGCATGGCGCGAGCGCGCCTGAGCGGGTGGTGGTCGACGTCACCGCCG
CGAGCCCGAGCCCGTTGGACGTGGCGGGGTCGTCGCATGAGGCGACGAGGCAGGCGCTGT
CGCTGCTGCAAGCGTGGCTGTCGGAGCCGCGGCTCGAAGCGACCGAGCTGGTGTGGATCA
CGCGGGGCGCGGTGGGCGCGGCGCCAGACGACGCCGTCGAGGACCTGGCGCGCGCGCCGC
TGTGGGGGCTTGTCCGCGCGGCGCGAAGCGAGCACCCCGAACGCGGGCTGCGCTTGATGG
ATGTGGGGACCGAGCCCGTGGACGCTGGGCTGCTGGCGCGGGCGCTGGCGACGGCGGCGG
AGCCGGAGCTTGCGCTGCGCGGGGGCGCTGCGCTGGCCGCGCGCCTGGTGCGCGCACAGG
CGGTAGCGGAAGAGCTCACCCGAGCCCGCGAGCTGGACCCTGCGGGCACGQTGCTGGTGA
CCGGCGGGACAGGGGAGCTGGGTCAGGCGGTCGCGGCGCACCTGGTGCGCGCGCACGGGG
TGCGGCACCTTGTGCTGACGTCGCGGCGCGGGCTGGAGGCGCCCGGGGCCCGCGAGCTTG
TGGAATCGCTCGCGGAGCTCGGCGCCGAGACGGTGACGGTGGCCGCGTGCGACGTGTCGA
AGCGGGAGGAGGTCGCGCGTGTGCTGGCCGGCATCGACGCGGCGCGCCCGCTGAGCGCGG
TGCTGCACCTGGCCGGCGCGCTCGACGACGGCGTGCTCGCCGGCCAGACGGCCGAGCGCC
TCTCGCGGGTGCTGGCGCCGAAGGTGGACGGGGCGCTGCACCTGCACGAGCTGACGCGGG
AGCTGGATCTCGTGGCGTTCGTGCTGTTCTCGTCGGTGGCCGGTACGTTTGGCACGGCGG
GCCAGAGCAACTACGCGGCGGCGAATACGTTCCTCGACGCGCTCGCGGCGCACCGGCGCG
GCTGCGGGCTCGCGGCGACGAGCCTGGCGTGGGGGTTGTGGGCGCAAGCGGGCGTGGGGA
TGACAGCGCACCTCGGCGAGGCCGAACTGTCGCGCATCAGGCGCGCAGGGCTCGTGCCGA
TATCCGTCGACGAGGGCCTCGCTCTGCTGGACGCCGCGCTCTCACGCTCTGAAGCGAGCC
TGGTCCCAGTGCACCTGGATCTCGCGCAGCTGCAGCGTGGGCTGGAGTCCAGCGGCGAGC
TGCCGGCGCTGCTTCGCGCGCTGGTGCGCCCCGGGCTGCGCAAGGCGTCCTCTGCCGCGA
GGAAGGAGGCGTCGACGCTCCGCGAGCGCCTCTCGGCGCTGCCGGAGGCGGAGCGCCTGA
GCTCGCTCATCGACCTTGTGCGGGCCGAGGTCGCCGCGGTGCTCGGGCTTCAGCGCGGTG
ACGCGATTCCTACGGCCCAGCCCTTGAGGGAGCTCGGAATGGATTCGCTCATGGCCGTCG
AAGTCCGCAATCGGCTCGCCTTGCTGGTCGGAAGCAACTTGCCTGCCACTTTGCTTTTCG
ACCATCCATCTGCCACGCACATCGCGAAGTTCCTCCTGTCAAAGTTCGGAAACGGTGAGC
GCCGGAATCTGCTGCGTACAGCGGACTCCATGTCCGACGAGGAAATTCGCGCGTTCATGC
TCAGCCTCTCCGTCAGTCTCGTGCGTCGTTCAGGCCTCCTCCCCAAGCTCTTGGAGCTGC
GGGGGCCGTCCGAAACATCCGTCGAGGTTCCGGTTCCCATTTCCGATTTCGAAGATCTCG
CCGACGAGCAGCTGGCCTTGCAAGCCTTGCAAATGATTTCGAACTCAGAGGATCTCCATG
AATAG
TmbA
VAPMDRMLGFLRDALADLLQVAPGAVQDDVRFHEQGLDSARALLLVEKLSSWTGRALPAT
LVWQHSTLNALARHLADTSGEPISTALGAPAIASSSIDEPVAIVGLGCRMPGGIASPEGM
WRALCSGVDAIVEVPPDRWDAEAWLDADAQAPGKMTTRWGGFLNEIVSFDAGFFGISPAE
ARQMDPQQRFALETAWAALEDARVVPTALGGSRTGVFFGAMWQEYARVAGADATGTEPHS
AVGWDNSIIPARIAYALGLRGPVMTVNTACSSSLVAVHLAALSLRRGEADLALAGGVNLM
LTPHATVQMTKFGANSPDGRCRAFAAGANGYVRGEGCGVVVLRRLSDALVAGDRIYAVLR
GSAVNSDGASNGLTAPNPEAQADVIRDAWQPAGVAPREVAYVETHGTGTILGDPIEAAGL
AAVFAPGREQPLRIGSVKTNFGHLEAAAGIAGLLKATLALHHGQLPPNLHFESPNPHIDF
QALRLEVVARQQGWPRDAGRLAGVSSFGFGGTNAHVALEESPYRPQIINPLAADGEQALR
DAARRLLADFAASGAGRDAERLVVQPGTGSVRAMVQGRDGASVEAALRELADGGAELRPI
SRPRPRLVFFFSGHGGHWAGMVRDLMAGEPVFRATLEACDPAVRQFTGWSVIAELAANEA
CSRLDRTDVVQPVVFSVQVALARTLQSWGVVPELVFGQSVGEVAAAVVAGALSLRDGARV
ISTWSALIANHASGRGAMIVADLSVAQAERVLASWDAGGVQVAVHLAPGQVCIAGPVDAV
ERLVRRFASDGIGTMRVRIDYAAHTAEMEPLLPEIERQLAAIQPSAPTIPMWSTVANRYV
AGPELDGHYWARNLREPVQLVEAVESVARQHSACIVEVGPHPVAVRSFEASLKTWGDSRS
VVLATCWREVAARGGLEALLGALWREGIDVDWDAVRTGGPAATPPAVTLPLVVSGKTVQA
LQENAARLATHLDGRGDMALVDVAFTLATAPAHFATPASVQAASVSEAVEGLPALSEGPA
AAGVVRGTGVHGGKLAVLFTGQGSQRLGMGKSLYEACPVFPAAFDEVCEALDAHLDRGLR
EVVFAEAGSEQEALLERTEYTQPGLFALEVALYRQWESWGLKPAALLGHSIGELSAAHVS
GVLSLADAAKLVCARGRLMQRCQAGGAMMSVEASEPEVQGALSANGLEGVLGVAGINGPS
QTVLSGDEAAVLEVGRRFEAQGRRTRRLRVSHAFHSPHMDGMLEEFGKVARECAYGMPQV
PVVSSVTGELWSEQELRSPEYWVRQAREAVRFFDGMRTLAAAGVSTYVECGPDGVLCALG
AGCLPEGAEATFVASLRREQEEEPALATAVATVHVQGHEVDWAQVLSGHGGRPVELPTYA
FQRQRYWLEAPRARGDVGSAGLKAAAHPLLGAATKLADGEGHLFTGRLSLAEHAWLRDHQ
VFGRVVFPGTGMLELALAAGPAVGSRSLSELTLAEPLVLAEEGATRLQLSVGAPDTAGRR
EVGLYSQSEQAPEDAPWVQHATGVLTDETPGAPGELDELSTWPVPCAEEVDLSGVYERLR
ERGLDYGPAFQCLVELWRRGTRLYGRVVLPESAMDSAEAYGVHPALMDAALHALFAASSE
AAGADMALLPFSWSDVALHATGPRELRVMLELEGDMTQAVASLWFADAAGQAVASVGALH
LRRATAEQLAVASRANAQQLYRVDFQPVSVVASVPEAGSLVVVGAPEGGARLAEALGAEA
IADLDALVARLEQGASAPERVVVDVTAASPSPLDVAVSSHEATRQALSLLQAWLSEPRLD
AVELVWVTRGAVGAAPDDAVQDLAPAPLWGLVPAARSEHPERRLRLIDVGTEPVDAGLLA
RALAMAAEPELALRGGAALAARLVRVQAAAKELTQARGLGPAGTVLVTGGVGGLGQIVAT
HLVPAHCVRHLVLTSRRGLEAPGARELVRSLAELCAETVTVAACDVSKREEVARVLAGID
AARPLSAVLHLAGVLDDGVLAAQTAERLSRVLAPKVDGALHLHELTRELDLAAFVLFSSV
AGTFGTAGQSNYAAANTFLDALAAHRRGRGLAATSLAWGFWAPGAGGMTDTLGEALESRI
RRAGFIPMYVAEGLFLLDVALSRSEASLVPAHFDLGKLRSGLDASGGVPALLRALVRPSL
RKASSGTQETSALRARLSALPESARLEALIDLVRGEVAAVLGLHHPALVEPHQVLKEVGL
DSLMAVELRSRLSSREATSLPATLAFDHPTPPATAELLLKQAFSELQETGGGARVRRRVR
EDEPIAIVSMACRLPGGIETPEDFWRVLREGKDVTESFPSRYEASSVYDPDPEAVGKSYA
REGGFLRNVDLFDAGFFGTSPREAQSMDPQQRLVLETAWEALERAGVRPSVLSESATGVY
LGWMGSDYAAGHADDLRTLDGYRITGGAGSVISGRVAYVLGLQGPATTVDTACSSSLVSL
HLACMALRRGECDLALAGGVMVMTTPATFVEFSRLKGIARDGRCKSFSAQADGVIWAEGC
GMLVLKRLSDARRDGDRVLAVVRGSAVNQDGRSQGLTAPNGPAQQRVIQQALSSCGLSPE
DIDAVEAHGTGTSLGDPIEAGALAEVFGPGRKAERPLYLGSSKSNLGHAQSAAGVAGVIK
MVLSMQHEVLPRTLHAEQPSPHIGWEGSGLSLLQEARPWRRNGRARRAGVSSFGISGTNA
HVILEEAPVEAVREPVAAEAAEGAAMPLLLSGRDQASVAAQAGRWAKWLEEHAEVGWSDV
VRTAALHRTHFTTPASVLAASAAEAVEGLPALSEGRAAAGVVRGTGVRGGKLAVLFTGQG
SQRLGMGKRLYEVYPVFRAAFDEVCEALDAHLARGLREVVFAAAGSEEGALLERTEYTQP
GLFALEVALYRQWESWGLKPAALLGHSIGELSAAHVAGVLSLADAAKLVCARGRLMQGCE
AGGAIVSVEASEPEVQRALSEVGAQGRLSIAGLNAPMQTVLSGDEAAVLAVARRLEAQGR
RTRRLRVSHAFHSAHMDGMLEEFGKVARECTYARPQLAVVSGVTGELGGEEALMSAEYWV
RQVREAVRFLDGMRTLAAAGVSTYVECGPDGVLCALGAGCLPEGAEATFVASLRREQEEE
RALATAVATVIVQGHEVDWARVLSGQGGRAVELPTYAFQRQRYWLEAPPARGDVGSAGLK
AAATPLLGAATKLADGEGHLFTGRLSLAEHAWLRDHGVFGQVVFPGTGMLEVALAAGPAV
GSRSLSELTLAEPLVLAEEGAARLQVMIGAPDAAGRREVGLYSQSEQAPEDAPWVQHATG
VLTDEPPGAPGELDELSTWPVPGAEEVDLSGVYGRLREPGLDYGPTPQGLVELWRRGTRL
YGRVVLPESAMDSAEATGVHPALMDAALHALFAASSEAAGADMALLPFSWSDVALLATGP
RELRVMLELEGDMTQAVASLWVADAAGQAVASVGALHLRRATAEQLAVASRANAQQLYRV
DFQPVSVVASVPEAGSLVVVGAAEGRGRLAEALGAEAIADLDALVARLEHGASAPERVVV
DVTAASPSPLDVAVSSHEATRQALSLLQAWLSEPRLDDVELVWMTRGAVGAAPNDAVEDL
AHAPLWGLIRTARSEHPERRLRLIDVGTEPVDAGLLAPALATAAEPELALRGGAALAARL
VRVQAAEELTRGARELDPAGTVLVTGGTGELGQAVAAHLVRAHPGVRHLVLTSRRGLEAP
GARELVQSLAELGAETVTVAACDVSKREEVARVLAGIDAARPLSAVLHLAGVLDDGVLSS
QTPERISRVFAPKVDGALHLHELTRELDLAAFVLFSSVAGTFGTAGQSNYAAANTFLDAL
AAHRRGGGLAATSLAWGFWTQAGVGMTAHLGKAELSRMRRNGFVPMPVEEGLALLDAALL
RSEASLVPVHLDLAQLQRGLESSGELPALFRALLRPSLRKASTATATRRDASALRGRLSA
LPEAERLNALTELVRGEVAAVLGLQRSEAVGAEQVLKGLGLDSLMAVELRNRLAARAETS
LPATLVFDYPTPRAIAELLLKQAFSGLQVKEARARRHAGKDEAVAIVSMACRLPGGVETP
EDYWRLLAEGKDATBGLPARWEALSVYDPDPEAVGKSYAREGGFLRDTDLFDADFFGISP
REAQSMDPQQRLVLETAWEALEPAGVRPSALSGSATGVYLGSMGSDYGALHTGGLEALDG
YRGTGSAASVLSGRVAYVLGLQGPAITVDTACSSSLVSLHLACTALRQGECDLALAGGVT
VMSTPALFVEFSRLKGMARDGRCKSFSARADGVTWSEGCGMLVLKRLSDARRDGDRVLAV
VRGSAVNQDGRSQGLTAPNGPAQQRVVQRALSSCGLSPEDIDAVEAHGTGTSLGDPIEAG
ALAEVFGPGRKAERPLYLGSSKSNLGHTGPAAGVVGVLKMVLSMQHEVLPRTLHAEQPSP
HIGWEGSGLSLLQEARPWRRNGRARPAGVSSFGISGTNAHVILEEAPVEAAREPVEAVRE
PLATEGVANPLLLSGRDEASVAAQAERWAKWLEEHAEVGWSDVVRTAALHRTHFASPASV
LAASVSEAEEALRALSQGRGHRAVSAGTARARGKVVFVFPGQGSQWPGMGRALLEQSAAF
AEAVQACDEALRPWTGWSVLSVLRGEAGEAGEEQPSIJERVDVVQPALFAMCVGIAAWRS
LGLEPAAVVGHSQGEVSAAVVCGALSLAEGARVVALRSQAVRQRSGMGAMMLVERPVSEV
QERIAPYGEALAIAAVNTSSSTVVSGDVEAVDGLMGELTAEGVFCRKVNVDYASHSAHMD
ALLPELGAKLSSLRPKATQLPFYSTVAGEVSRGEALDGEYWCRNLRQTVRLDRALSKLLE
DGHGVFVEVSAHPVLAMPLTTACGEAQGVVVGSLQRDEGGLSQLYRTLGQLHVQGHEVDW
ARVLPGHGGPAVELPTYAFQRQRYWLEAPPARGDVSSAGLKAAAHPLLGAATKLADGEGH
LFTGRLSLAEHAWLRDHGVFGQVVFPGTGMLEVALAAGPAVGSRSLSELTLAEPLVLAED
GAARLQVMIGAPDAAGRREVGLYSQPEHAPEDAPWVQHATGVLTDELPGIPDELDELSMW
PVPGAEEVDLSGFYERLRERGLHYGPTFQGLVELSRQGTRLYGRVVLPGTEKDRAEAYGL
HPVLMDAALQVLGAAGEGHWEADALFMPFSWADAATHATGPSELRVRVELEETDGSTQAT
ASLCAADAAGQPVASVGALRLRRVTAEQLPAVTRTDEQHLYRVSFQPVSLAQAPLEAGSL
VVLGAAEGRGQLADTLGAEAIADLDALRAWIERGAPTPVRVVIDTNAASSPRSDVAGSSH
EATRQALSLLQAWLSEPRLDAVELVWVTRGAVSAAPDDAVEDLAHGPLWGLIRTARSEHP
ERRLRLIDVGTEPVDAGLLARALATAAEPELALRGGAVLAARLVRVQAAAEELTPARGLD
PAGTVLVTGAVGGLGQAVTRHLVRAHGVRHLVLTSRRGLEAPGARELVQSLEELGAETVS
MVACDVSKREEIARVLAGIDAARPLSAVLHLAGVVHDGVIQTQTAERLAWVLAPKVDGAL
HLHELTRELDLAAFVLFSSAAGTLGMAGQGNYAAANTFLDAFAAHRRGRGLAATSLAWGV
WTPAGGGMAAQLGAAELARFSRYGVVSMSVEEGLSLLDAALSRPEASLVPMHLDLAQLQR
GLEANGELPALFRALLRPSLRKASTATRRDASALRGRLSALPEAERLNALIELVRGEVAA
VLGLQRSEAVGAEQVLKGLGLDSLMAVELRNRLAARTETSLPATLVFDYPTPRAIAELLL
KLAFSGPQVMGARRGVRRHAGKDEAVAIVSMACRLPGGVETPEDYWRLLAEGKDVIEGLP
ARWETLSVYDPDPEAVGKSYAREGGFLRDIDLFDADFFGISPREAQAMDPQQRLVLETAW
EALERAGVRPSALSGSATGVYLGAAGSDYGAYQGGGLEMLDGYRGIGSAASVLSGRVAYV
LGLHGPAMTVDTACSSSLVSLHLACTALRQGECDLALAGGVTVMSTPALFVEFSRLKGMA
RDGRCKSFSGQADGAGWSEGCGMLVLKRLSDARRDGDRVLAVVRGSAVNQDGRSQGLTAP
NGPAQQRVIQQALSSCGLSPEDIDAVEAHGTGTSLGDPIEAGALAEVFGPGRKAERPLYL
GSSKSNLGHAQAAAGVAGVLKMVLSMQHEVLPKTLHAEQPSPHIGWEGSGLSLLQEARPW
RRNGRARRAGVSSFGISGTNAHVILEEAPVEAAREPVEAVREPVEAEGVAIPLLLSGRDE
ASVAAQAGRWAKWLEEHGEVGWSDVVRTAALHRTHFESRASVLAASAAGAVEGLRALSSG
RPDAAVVSGTAKRGGKLAVLFTGQGSQRLGMGKRLYEVYPVFRAAFDEVCEALDAHLDRG
LREVVFAAAGSEEGALLERTEYTQPGLFALEVALYRQWESWGLKPAALLGHSIGELSAAH
VAGVLSLADAAKLVCARGRLMQGCEAGGAMVSVEASEPDVQRALSEVGAQGRLSIAGLNA
PMQTVLSGDEAAVLAVARRLEAQGRRETTLRVSHAFHSAHMDGMLEEFGKVARGCTYARP
RLAVVSGVTGELGGEEALMSAEYWVRQVREAVRFLDGMRTLAAAGVSTYVECGPDGVLCA
LGAGCLPEGAEATFVTSLRREQEEERALATAVATVHVQGHEVDWAQVLSGRGGRPVELPT
YAFQRQRYWLEAPKTTAAQANVSWPERALWDAVQKGEGVADLLELPDDVRESVAPLLPYL
AAWRRRRDAEATVSGWLYEEAWQREASAARGKPDVRGRWLLVSSPRAGGLTAAVSDALGA
AGAEVIIEPATEERAQLAARLRGLEGELRGVVALSAPGEQGALEEGRGPRGVYEVLALAQ
ALGDAGLDARLWVLTQGAVSTEASEGVSDPAQALTWGLGRVVGLEHPERWGGLVDLPAEV
DAEAVQQVLRTLVAEDHEDQVAVRRGGRLVRRIVRVSGEDGGAGWKPRGTVLITGGVGGL
GSHLARWLAERGAEHLVLASRRGAAAAGARELREELEGRGARVTLAACDVSERAQVEALV
RELEQDEAPLSAVAHLAGIVRRVPVRELAPEMLAQELAAKVNGAWHLQELLAERELDAFV
LYGSIAGLWGSGTQAGYGAANAGLDALARYRRARGQTATVLHWGPWSGGGMVSDEAEPQL
RSRGLVPMSPDKALCGLEVGLRRTSVAIADVDWSRFAPLFCAARPRPLLYGIEQARHALE
GRTPQQAAGGAGDKALREMLLGLPAVERSERLRELVASETAAVLGVKDPSGLDPERGFLD
LGLDSLMAVELSKRLQQRTGVSVTRTLIFDYPTQGEVTRWLLEQLMPPERPAADEHGVSR
GPERSAPIAIVGVGLRMPGGANDLESFWQVLVEGRDTLRPIPTDRFDVEANYDPNPEAKG
KTYVKHASLLDDVASFDAGFFGISPREAEPMDPQHRLLLETAWSALEDAGVRPDQLKGSD
TGVFVGVAPSEYASYRGKSANEDAYALTGTALSFAAGRVAYHLGLQGPAVSVDTACSSSL
VALHLACDALRRGDCEVALAAGVQVLANPAGFVLLSRTRAVSPDGRCKTFSQAADGYGRG
EGVGVVVLMRLSDAQAQGMRVLGVVRGTAVNQDGASSGITAPNGTAQQKVVRAALRNAGL
EASSIDVVECHGTGTSLGDPIEVQALGAVYGQGREATRPLRLGAVKSNIGHLESAAGIAG
VCKILAAFRHEALPATLHSSPRNPQISWESLPVQVVDRLTGWPRPADGLPRFAGVSSFGI
SGTNAHVILEEAPLEAVREPAAVREPLAAEGVAIPLLLSGRDEASVGAQAERWAKWLGEH
AEVRWPDVVRTAALHRTHFAWRASVQAASVSEAVEGLRALSEGRAAAGVVRGTGGRGGKL
AVLFTGQGSQRLGMGKRLYEVYPVFRAAFDEVCEALDAHLDRGLREVVFAEAGSEQEALL
ERTEYTQPGLFALEVALYRQWEAWGVRPAALLGHSIGELSAAHVAGVLSLADAAKLVCAR
GRLMQRCQAGGAMMSVEASEPEVQGALSAMGLEGRLGIAGINGPSQTVLSGDEAAVLEVG
RRFEAQGRRTRRLRVSHAFHSAHMDGMLEEYGRVARECAYGRPQVPVVSGVTGELGGEES
LMSAEYWVRQVREAVRFLDGMRTLAAAGVSTYVECGPDGVLCALGAGCLPEGAEATFVAS
LRREQEEERALVTAVATVHVQGHEVDWAQVLSGHGGRPVELPTYAFQRQRYWLEAPRARG
DVGSAGLKAAAHPLLGAATKLADGEGHLFTGRLSLAEHAWLRDHQVFGKVVFPGTGMLEL
ALAAGRAVGSRTLSELVLAEPLVLAEEAAARLQLSVGAPDAAGRREVGLYSQSEQAPEDA
PWVQHATGVLTDEIPGAPGELDELSTWPVPGAEEVDLSGFYERLREGGLDYGPVFQGLVE
LWRRGARLYGRVVLPGSARGSAEAYGVHPALMDAALHTMVAAFSQMSGPDGVLLPFAWSD
VAPHATGASELRIRVEMQEQSAQQPAASLYVADCTGQVVASIGALRLRRATAEQLRTAVH
AGGQHMYQVSFQPVDLAAPPLVTGSLVVIGAPKGGARLAEALGAEAIADLDALVVRLEHG
ASAPERVVVDVTAASPSPLDVAGSSHEATRQALSLLQAWLSEPRLEATELVWITRGAVGA
APDDAVEDLARAPLWGLVRAARSEHPERGLRLMDVGTEPVDAGLLARALATAAEPELALR
GGAALAARLVRAQAVAEELTRARELDPAGTVLVTGGTGELGQAVAAHLVRAGGVRHLVLT
SRRGLEAPGARELVESLAELGAETVTVAACDVSKREEVARVLAGIDAARPLSAVLHLAGA
LDDGVLAGQTAERLSRVLAPKVDGALHLHELTRELDLVAFVLFSSVAGTFGTAGQSNYAA
ANTFLDALAAHRRGCGLAATSLAWGLWAQAGVGMTAHLGEAELSRIRRAGLVPISVDEGL
ALLDAALSRSEASLVPVHLDLAQLQRGLESSGELPALLRALVRPGLRKASSAARKEASTL
RERLSALPEAERLSSLIDLVRAEVAAVLGLQRGDAIPTAQPLRELGMDSLMAVEVRNRLA
LLVGSNLPATLLFDHPSATHIAKFLLSKFGNGERRNLLRTADSMSDEEIRAFMLSLSVSL
VRRSGLLPKLLELRGPSETSVEVPVPISDFEDLADEQLALQALQMISNSEDLHE
tmbB bases 31550-52471
ATGAATAGCAGCGCCGCCTCTCCTACGCTTCGTGAGGCGTTGACCCGTGCATTGAAAGAG
TTGCAGAGGCTGCAGGCCAGCCACTCGGATCTCCGTTCAGGGCCCATCGCCATCGTATCG
ATGGCGTGCCGGCTGCCGGGGGGCGTCGCTACGCCGGAAGACTACTGGCGTCTCCTGGAG
GAGGGGAGAGATGCGATCGAGGCCTTCCCTGCGCGCTGGGATGCACCTTCGATTTACGAC
CCCGATCCGGAGGCGGTGGGCAAGACCTACGTGCCCGAGGGTGGATTCCTGCGGGACATC
GACCTGTTCGATGCGGGTTTCTTCGGGATATCGCCCCGAGAGGCGCAGGCGATGGATCCC
CAGCAGCGGCTGGTGCTGGAGACGGCGTGGGAGGCACTGGAGCGGGCTGGCGTGCGCCCG
TCGGCGCTGAGCGAGAGCTCCACCGGGGTGTACCTGGGCTCGATGGGCTCGGACTACGGT
GCTCTTTACGGCAGCGACCTGGCGGCGTTGGACGGCTACCGGGGCACCGGGAGCGCGGCG
AGCGTGCTCTCAGGCCGTGTGGCGTACGTGCTCGGGCTGCAGGGCCCAGCGATCACGGTG
GACACGGCGTGCTCGTCGTCGCTGGTGTCGCTGCACCTGGCGTGCACGGCCCTGCGTCAG
GGCGAGTGCGATCTGGCCTTGACCGGTGGGGTGATGGTGATGACCACACCCGCGGGATTC
GTGGAGTTCAGCCGCCTCAAGGCCCTTGCACGGGACGGTCGTTGCAAGAGCTTCTCTGCG
CGAGCTGACGGCGTCATCTGGTCCGAGGGGTGCGGGATGCTGGTGCTGAAGCGGCTGTCG
GACGCGCGGCGCGACGGTGACCGTGTGCTGGCGGTGATCCGTGGGTCAGCGGTGAACCAG
GACGGTCGCAGCCAGGGTTTGACGGCGCCGAACGGCCCTGCCCAGCAGCGGGTGATCCAA
CAGGCGCTCTCGTCGTGCCGGCTGTCGCCCGAGGACATCGACGCGGTGGAGGCGCATGGG
ACGGGCACGAACCTGGGCGACCCGATCGAGGCCGGAGCGCTCGTGGAGGTGTTCGGGCCT
GGGCGCAAGGCCGAGCGGCCGCTGTACCTTGGCTCGTCGAAGTCGAACCTGGGACACGCG
GGGCCTGCGGCGGGCGTCGCCGGTGTGCTCAAGATGGTGCTGTCGATGCAGCACGAGGTG
CTGCCGCGGACGCTGCACGCGGAGCAGCCGAGCCCGCACATTGGGTGGGAGCGGAGCGGG
CTGTCGTTGCTGCAAGAGGCGCGTCCGTGGCGGCGCAACGGCCGGGCGCGGCGCGCGGGC
GTGTCGTCGTTCGGGATCAGCGGGACGAACGCGCATGTCATCCTCGAAGAGGCGCCGGTG
GAGGCGGCGCGCGAGCCGGTGGAGGCAATGCGCGAGCCGTTGGCGACGGAGGGTGTTGCG
ATGCCGCTGTTGCTGTCGGGGCGAGACGAGGCCTCGGTGGGGGCGCAGCCGGAGCGCTGG
GCGAAGTGGCTCGGAGAGCACGGGGAGGTGCAGTGGTCGGACGTGGTGAGGACAGCGGCG
CTGCACCGGACGCACTTCGCCTCACGCGCATCGGTGCTTGCGGCGAGCGTGTCCGAGGCG
GAGGAGGCGCTGCGGGCGCTGTCGCAGGGTCGCGGCCACCGGGCGGTGTCGGCGGGTACG
GCGCGTGCGCGAGGCAAGGTGGTGTTCGTGTTCCCCGGCCAAGGGAGCCAGTGGCCGGGG
ATGGGCCGGGCGCTGCTGGAGCAGAGCGCGGCGTTCGCGGAGGCGGTGCAGGCGTGCGAT
GAGGCGCTGCGGCCGTGGACGGGCTGGTCTGTGCTGTCGGTGCTGCGCGGAGATGGCGGG
GAGGAGCAGCCGTCGCTGGAGCGGGTGGACGTGGTGCAGCCCGCGCTGTTCGCGATGTGC
GTGGGTCTGGCCGCGGCGTCGCGGTCGCTGGGGCTGGAGCCTGCGGCGGTGGTGGGCCAC
AGCCAGGGCGAGGTGTCGGCGGCGGTGGTGTGCGGAGCGCTGTCGCTTGCGGAGGGAGCG
CGGGTAGTGGCGCTGCGCAGCCAGGCGGTGCGGCAGCGGTCGGGGATGGGGGCGATGATG
CTGGTCGAGCGGCCGGTGTCGGAGGTGCAGGAGCGCATCGCGCCGTACGGGGAGGCGCTT
GCGATAGCGGCGGTGAACACGTCGAGCTCGACGGTGGTGTCGGGTGACGTGGAGGCGGTG
GACGGGCTGATGGTGGAGCTGACGGCAGAAGGTGTGTTCTGCCGGAAGGTGAACGTCGAC
TACGCGTCGCACAGCGCGCACATGGATGCGCTGCTGCCCGAGCTAGGAGCGAAGCTGTCG
TCGCTCAGGCCGAAGGCGACGCAGCTGCCGTTTTACTCGACGGTGACAGGAGAGGTGTCG
CGGGGCGAGGCGCTGGACGGCGAGTACTGGTGCCGCAACCTTCGGCGGACGGTGCGCCTG
GACCGAGCGCTGTCGAAGCTGCTGGAGGACGGGCACGGTGTGTTCGTGGAGGTGAGCGCG
CACCCGGTGCTGGCGATGCCGCTGACGACGGCGTGCGGGGAGGCGCAGGGGGTGGTGGTG
GGGAGCCTGCAGCGCGACGAAGGTGGGTTGTCGCAGCTGTACAGGACGCTGGGGCAGCTG
CACGTGCAGGGGCACGAGGTGGACTGGACACGGGTGCTGTCGGGCCACGGCGGTCGTGTC
GTGGAGCTGCCGACGTACGCGTTTCAGCGGCAGCGTTACTGGCTGGATATCTCGAAGGCG
CGTAGCGACGTGAGCTCGGCGGGGCTCAAGGCAGCAGCACACCCGCTGCTCGGCGCCGCG
ACGAGGCTCGCCGACGGCGAGGGGCACCTGTTCACAGGGCGGCTGTCGCTGGCGGAGCAT
CCGTGGCTTCGGGATCATGAGGTGTTTGGCCAGGTGGTGCTTCCGGGCACGGGGACGCTG
GAGCTGGTGCTGGCGGCGGGGCGCGCGGTGGGCAGCCGGTCGCTGTCGGAGCTCACGCTG
GCCGAGCCGCTGGTGCTGGCCGAGGGCGCGGCGCGGCTGCAGGTGATGATCGGAGCGCCG
GACGCGGCGGCCCGGCGCGAGGTGGGGCTGTACAGCCAGCCTGAGCAGGCCCCGGAGGAC
GCGCCGTGGGTGCAGCACCCGACGGGAGTGTTGACGGACGAGCCCCCCGGCATCCCTGTC
GAGCTCGACGAGCTGTCGACGTGGCCTGTGCCGGGCGCGGAGGAGGTGGACCTGTCCGGG
CTTTACGAGCGGCTGCGTGAGCGCGGGCTCCACTACGGCCCGGCGTTTCAAGGGCTCGTG
GAGCTGTCGCGCCAAGGCACAACCTACTTCGGTCGGGTGGTGCTGCCGGGGACCGAGAAG
GACAGGGCGGAGGCGTATGGCGTGCACCCGGCGTTGATGGACGCCGCGCTCCACACGATG
GTCGCGGCCTTCTCCGAGAGCCCAGGGGCGAACGAGGTGCTCGTGCCGTTTGCCTGGTCG
GACGTGGCGCTGCACGCGACGGGGGCGAGCGAGCTTCGGGTCCGGGTAGAGCTCCAGGAC
GGAGGCGCACACCAGGACACCGCTTCGCTGCAAGTCGCAGACTCCACGGGGCAGGCCGTG
GCGAGCATCGGCGCTCTACATCTCCGCCGGGCGACGGCCGAGCAGCTGCGGACCGCCGTT
CACGCTGGTGGCCAACATATGTATCAGGTGAGCTTCCAGCCTGTGGAGCTCGCGGCAGCC
CCCCTGGAGGCGGGCTCGCTGGTGGTCGTCGGTGCAGCGGAGGGACGAGGCAGGCTGGCC
GAAGCCCTGAGGGCGGAGGCGATTGCCGATCTCGAAGCATTGGTTGCGCGCCTCGAGCAG
GGCGCGAGCGCGCCTGCGCGGGTGGCGGTCGACACGACAGCTTTAGGACAGAGTCAGTCG
GGAGTGGCGTCGTTGTCCCACGAGGCGACGAGGCAGGCGCTGTCGCTGCTGCAAGCGTGG
CTGTCGGAGCCGCGGCTCGACGCTGTCGAGCTGGTGTGGGTGACGCGGGGCGCGGTGGGC
GCGGCGCCGGACGACGCCGTCCAGGATCTGGCGCGCGCGCCGCTGTGGGGGCTTGTTCGC
GCGGCGCGCAGCGAGCACCCCGAGCGCCGGCTGCGCTTGATCGATGTGGGGACCGAGCCC
GTGGACGCTGGGCTGCTGGCGCGGGCGCTGGCGACGGCGGCGGAGCCGGAGCTTGCGCTG
CGCGGGGGCGCTGCGCTGGCCGCGCGCCTGGTGCGCGCGCAGGCGGCAGCGGAAGAGCTC
ACCCGAGGAGCCCGCGAGCTGGACCCTGCGGGCACGGTGCTGGTGACCGGCGGGACAGGG
GAGCTGGGTCAGGCGATCGCGGCGCACCTGGTGCGCGCGCACGGGGTGAGGCACCTTGTG
CTGACGTCGCGTCGCGGGCTGGAGGCGCCCGGGGCCCGCGAGCTCGTGCAGTCACTCGAG
GAGCTCGGCGCCGAGACGGTGACGGTGGCTGCGTGCGACGTGTCGAAGCGGGAGGAGGTC
GCGCGTGTGCTGGCCGGCATCGACGCGGCGCGTCCGCTGAGCGCGGTGCTGCACCTGGCC
GGCGTGCTCGACGACGGCGTGCTCACCGCCCAGACGGCCGAGCGCCTCTCGCGGGTGCTG
GCGCCGAAGGTGGACGGGGCGCTGCACCTGCACGAGCTGACGCGGGAGCTGGATCTCGCG
GCGTTCGTGCTGTTCTCGTCGGCGGCCGGTACGTTTGGCGCGGCGGGCCAGAGCAACTAC
GCGGCGGCGAACACGTTCCTCGACGCGCTCGCGGCGCACCGGCGCGGCGGCGGGCTCGCG
GCGACGAGCCTGGCGTGGGGCTTCTGGACACAGGCGGGCGTGGGGATGACAGCGCACCTG
GGCGAGGCCGAGCTGTCGCGCATGAGGCGCAATGGGTTCGTGCCGATGCCGGTGGAAGAG
GGCCTCGCTCTGCTGGACGCCGCGCTCTCACGCCCTGAAGCGAGCCTGGTCCCAGTGCAC
CTGGATCTCGCGCAGCTGCAGCGTGGGCTGGAGTCCAGCGGCGAGCTGCCGGCGCTGTTT
CGTGCGCTGTTGCGCCCGAGCTTGCGCAAGGCGTCCTCGGCAACGAGGCGAGACGCCTCG
GCGCTCCGCGAGCGCCTCTCGGCGCTGCCGGAGGCGGAGCGGCTGAATGCGCTCGTCGAG
CTGGTGCGGGGCGAGGTTGCGGCCGTGGCAGGGCTTCAGCGCGGCGAGGCTGTGGCAGCG
GATCAGGTGCTCAAGGAGCTGGGGCTCGACTCGCTGATGGCGGTGGCGCTGCGCAACCGC
CTCACGTCCCGTACCGAGACGTCCTTGCCGGCGACGCTGGTCTTTGACTACCCGACGCCG
CGGGCGATCGCAGAGCTGCTGCTGAAGCAGGCGTTCTCGGGGCTGCAGGTGAAGGAAGCG
CGGGCGCGGGTGCGTCGTCGTGCAGGGAAAGACGAGCCGATCGCGATCGTGTCGATGGCG
TGCCGGCTGCCGGGAGGCGTTGCGACGCCGGACGACTACTGGCGTCTCTTGGCCGAGGGG
AAGGACGCGATCGAAGGCCTCCCAGCGCGCTGGGACGGGTTCGAGGTCTACGACCCTGAT
CCGGAGGCCGCAGGCAAGAGCTACGCGCGCGAAGGTGGGTTTGTTCGGGACATCGACCTG
TTCGACGCGAACTTCTTCGGGATATCGCCGCGCGAGGCGCAGTCGATGGATCCGCAGCAT
CGGCTGGTGCTGGAGACGGCGTGGGAGGCATTGGAGCGTGCCGGCGTGCGCCCGTCGGCG
CTGAGCGGGAGCGCCACCGGAGTGTACCTGGGTTCGATGGGCTCGGACTACGGTGCTCTT
CATACTGTCGATCTGAAGGAGCTGGACGGGTACCGGGGCATCGGGAGCGCGGCGAGCATC
CTCTCGGGCCGGGTGGCCTACGCGCTGGGGCTGCAGGGCCCAGCGATGACGGTGGACACG
GCGTGCTCGTCGTCGCTGGTGTCGCTGCACCTGGCGTGCACGGCGCTGCGCCAGGGCGAA
TGCGACCTGGCGCTGGCCGGCGGGGTGACGGTGATGAGCACCCCCGCGCTGTTCGTGGAG
TTCAGCCGTCTCAAGGGGATGTCCCGCGACGGTCGGTGCAAGAGCTTCTCGGTGCAGGCG
GACGGCGCGGGCTGGGCCGAGGGTTGCGGGATGCTGTTGTTGAAGCGGCTGTCTGACGCG
CAGCGCGACGGCGACCGTGTGCTGGGGGTGATCCGTGGCTCTGCGGTGAACCAGGACGGT
CGCAGCCAGGGTCTGACGGCGCCGAACGGCCCTGCCCAGCAGCGGGTGATCCGGCAGGCG
CTGTCGTCGTGTGGTCTGTCGCCCGAGGACATCGACGCGGTGGAGGCGCACGGTACGGGC
ACGAGCCTTGGAGACCCGATCGAGGCCGGAGCGCTGGCGGAGGTGTTTGGACCGGAGCGT
AGCCCCGAGCGTCCGCTGTACCTGGGATCGTCGAAGTCGAACCTCGGACATGCGCAGGCG
GCGGCGGGCGTGGCGGGCGTGATCAAGATGGTGCTGTCGATGCAGCACGAGGTGCTGCCG
AAGACGCTGCACGCGGAGCAGCCGAGCCCGCACATTGGGTGGGAAGGAAGCGGGCTGTCG
CTGCTGCAAGAGGCGCGTCCGTGGCGGCGCAACGGCCGGGTCCGTCGTGCCGGCGTGTCG
TCGTTCGGGATCAGCGGGACGAACGCGCATATCATCCTCGAAGAAGCGCCGGCCGAGGCG
CGGCGCGAGCCTGTCGAGGCCGAGGCGGCGCCTGCGCTATTGCCGCTGGTGCTGTCGGGT
CGAGACGAGGCCGCGGTGAATGCGCAGGCGGGGCGGTGGGCGAAGTGGCTGGAAGAGCAC
GGGGAGGTGGGGTGGTCGGACGTGGTGCGCACGGCGGCGCTGCACCGGACGCACTTCGAG
TCGCGGGCGTCGGTGCTTGCGGCGAGCGCTGCGGGAGCTGTGGAGGGTCTTCGCGCGCTG
TCGTCGGGGCGGCCGGATGCGGCGGTGGTGAGCGGGACGGCGAAGCGAGGCGGGAAGCTT
GCGGTGCTGTTCACGGGGCAGGGCAGCCAGCGGCTCGGGATGGGGAAGAGGCTTTACGAA
GTGTACCCCGTGTTCCGTGCGGCGTTCGACGAGGTGTGCGAGGCGCTGGACGCGTATCTC
GACCGTGGGTTGAGAGAGGTGGTGTTCGCGGCCGCGGGCAGCGAGGAAGGAGCGTTGCTG
GAGCGGACGGAGTACACGCAGCCCGGGCTGTTTGCGCTGGAAGTGGCGCTGTACCGTCAG
TGGGAGTCGTGGGGGCTGAAGCCCGCTGCGCTGCTGGGGCACTCGATAGGAGAGCTGAGC
GCTGCGCATGTGGCGGGTGTGCTGAGCCTTGCGGACGCAGCGAAGCTAGTGTGCGCCCGA
GGTCGGCTGATGCAAGGGTGCGAGGCCGGGGGAGCGATGGTGTCGGTGGAGGCCTCGGAG
CCGGAGGTGCAGCGGGCGCTGTCGGAGGTCGGGGCGCAGGGGCGACTGAGCATCGCCGGG
CTGAACGCGCCGATGCAGACGGTGCTGAGCGGGGACGAAGCGGCGGTGCTCGCGGTGGCG
CGGCGGCTGGAGGCGCAGGGCCGGCGCACGCGGCGTCTGCGTGTGTCGCACGCGTTCCAC
AGCGCGCACATGGACGGGATGCTGGAGGAGTTCGGGAAGGTGGCGCGGGAGTGCACGTAC
GCGCGGCCACGGCTGGCGGTGGTGAGCGGCGTGACGGGCGAGCTCGGTGGCGAAGAAGCG
CTGATGTCGGCCGAGTACTGGGTGAGGCAGGTGCGCGAGGCGGTGCGCTTCCTGGACGGG
ATGCGCACGCTTGCGGCGGCGGGGGTGAGCACATACGTCGAGTGTGGGCCGGATGGCGTG
CTGTGCGCGCTGGGGGCGGGGTGCCTGCCGGAGGGAGCCGAGGCGACGTTTGTGACGAGC
CTGCGGCGAGAGCAGGAGGAAGAGCGCGCGCTGGCGACGGCGGTGGCGACAGTGCACGTG
CAGGGGCACGAGGTGGACTGGGCCCGGGTGCTGTCGGGCCGTGGCGGCCGGCCCGTGGAG
CTGCCGACGTACGCGTTCCAGCGGCAGCGCTACTGGCTGGAAGCGCCGAAGAGCGCGGCG
ACCGAGGTGAATGTCTCCAGCGCGGAGCAGGCGCTGTGGAATGCGGCGCTGGAGGGCGAG
GGAGATGGCGTTGCGGAGCTGCTGGAGCTGCCTGACGACGTGCGCGCCAGCGTCGGGCCG
CTGCTGCCGTACCTCGCGGCGTGGCGCCAGAGAAAGCAGGCAGAAGCGGCCGCGGCGAGC
TGGCTGTATGAGGAAGCGTGGCAAAACCGTCCGAGGCGTGTGACGGGTAGTCCGGATGTA
AGGGGCACCTGGCTCGTGGTGTCACCTCCGCTTGCCGGAGAGCTTGCGGAGGTGGTGCGT
GGTGCGCTCGGCGCCGCGGGGGCCGAGGTGATCGTGCACATCGCGGCCGTGGAGCGAGCG
CAGCTCGCAGCGTGGCTGAGAGAGCAAGCGCGCCTGAGAAAGGAGGAGGGCGAGCTGCGT
GGCGTCATCGCGCTCACGCCCTCAGGCGAGGAAGGCGCGCTGGAGCAAGGGCAGGCGCCC
CGCAGCCTGTACCAGACGCTGGCGGTGGTGCAGGCGCTCGGCGACGCCGAAATCGGCGCG
CGGCTGTGGTTGCTCACGCAGGGAGCGGTCAGCACCGAGCCAAGCGAAGCGGTGGTGCAC
CCGTTGCAGGCGCTGACGTGGGGACTGGGACGGGCGTTGGGGCTGGAGCACCCCGAGCGC
TGGGGCGGGCTGCTGGACGTGCCGGCGGAGCTGGACGCGGGCGTGATGCAGCACGTCTTG
ACCACGCTTGTGTCCGACGACAACGAAGATCAGGTGGCGGTGCGGCGCGGCGGGCGCCTC
GTGAGGCGCATTGTGCGTGTGCGCGGCGAGGGCGACGGCGAGGGCTGGAAGCCGCGCGGC
ACGGTGCTCATCACGGGCGGCGTGGGCGGGCTCGGAGGTCATCTTGCCCGCTGGCTGGCC
GGGCGTGGCGCAGAGCACCTTGTGCTGGCGTCGCGCCGCGGCGCGTCGGCGCCCGGGGCG
AGCGAGCTGCGGGACGAGCTTGTGGCGCGGGGCATTCGGGTGACGCTGGCGGCGTGTGAC
GTGTCGGAGCGCGCGCAGCTCGCCGCGCTGCTCGCGGAGCTGGAGCAGGATGAAGCGCCG
CTGAGGGCGGTGGCGCACCTCGCGGGCATAGGCCGCCGTGTTCCGCTGCGAGAGCTCGAG
CCTGAGCAGCTCGAGCAGGAGCTCGCGGCGAAGGTGAAGGGGGCGTGGCACCTGCATCAG
CTGCTGGGGAAGCGGGAGCTGGATGCGTTCGTGCTCTATGGCAGCATCGCCGGGCTGTGG
GGCTCCGGGGCGCAGGCTGGGTACGGCGCAGCGAACGCAGGGCTGGACGCGCTCGCGCGG
TACCGGCGCGCGCGAGGGCAGGCTGCGACGGTGTTGCACTCGGGCCCCTGGGCGGGAGAA
GGGATGGTGACCAGCGAGCTCGAGTCGCAGCTGCGGATCCGCGGGGTCGCGGTGATGTCG
CCCGACAAGGCGCTCGCCGGGCTGGAGATGGCGCTGCGGCTGGGGCGCACGTCGGTGGCG
ATCGCCGACGTGGACTGGTCGCGCTTCGCGCCGTCGTTCAGCGCGCCGAGGCCGAGGCCG
CTCCTGGACGGGATCGAGGAGGCCCGGCGGGCGCAGGAGAGCCGCGGCCCGCAGCCGGCC
GCAGGCGGGACCGCGCTGAGAGACACCTTGCTGGGCCTGAGCGAGGCCGAGCGGCGCGAG
CGGGTACGTCAGCTGGTGGCGAGCGAGACGGCCGCGGTACTGGGCATCACGGACCCGAGC
CGGCTTGACCCGGACCGTGGCTTTCTGGACCTCGGGCTGGATTCGCTGATGGCGGTGGAG
CTGTCGAAGCGGCTGCAGAAGCGCACGGGCATGACGGTACCGAGCACGCTGAGCTTGGAT
CACCCGACGCAGAGCGACGTGGCGCGCTGGCTGCTGGAGCAGCTCACACCTCAGCCGCGA
CCGGAGCCGGCGGTGCGCGAGGTGAGCCGGGAAGAGGGGTGGAGCACCCCGATAGCGATC
GTGGGCGTGGGGCTGCGCATGCCTGGCGGAGCGAGCGACCTGGAGAGCTTCTGGCAGGTG
CTGGTCGAAGAGCGGGATACGCTGCGGCCGATCCCGGCCCAACGATTCGACGTCGAGGCG
CTGTACGATCCTGACCCCGACGCGAAGGGCAAGACGTACGTGCGGAACGCGTCGCTGCTC
GACGACGTGGCGTCGTTCGACCCTGGGTTCTTCGGGATAAGTCCGCGGGAGGCGGAGCCG
ATGGATCCGCAGCACCGGCTGCTGCTGGAGACGGCGTGGAGCGCCCTGGAGGACGCGGGG
GTGCGTCCAGAGCACCTGAAGGGCTCGGACACGGGAGTGTTCGTGGGCGTGGCGCCGAGC
GAATACGCGAGCTACCGAGGAAAGAGCGCGAACGAAGATGCGTATGCGCTGACAGGGACG
GCGCTGAGCTTTGCTGCGGGACGGGTGGCCTACCACCTCGGGCTGCAAGGCCCTGCGGTG
TCGACCGACACGGCCTGCAGCTCGTCGCTGGTAGCGGTGCACCTGGCGTGCGACGCGCTG
CGCCGGGGCGATTGCGAGGTGGCGCTGGCGGCAGGTGTGCAGGTGCTGGCGAACCCGGCG
GGGTTTGTGCTGCTGTCGCGCACGCGCGCGTTGTCGCCGGACGGGCGGTGCAAGGCGTTC
TCGCAGGCGGCGGACGGTTATGGCCGTGGCGAGGGAGTCGGGGTGCTGGTGCTGATGCGG
CTGTCCGAGGCGCAGCAGCAGGGGAAGCGGGTCCTGGGTGTGGTGCGCGGCACGGCGGTC
AATCAGGACGQCGCGAGCAGCGCGATCACGGCGCCGAACGGCACGGCGCAGCAGAAGGTG
GTGCGCGCGGCGCTGCGGAACGCGGGGCTGGAGCCGGCGAGCATCGATGTGGTGGAGTGC
CACGGTACGGGCACGTCGCTGGGCGACCCGATCGAGGTGCAGGCGCTCCGCGCGGTGTAC
GGGCAAGGTCGGGATATGGCTCGTCCGCTGCAGCTGGGCGCGGTCAAGAGCAATATCGGT
CATCTCGAGTCCGCCGCGGGCATCGCAGGGGTGTGCAAGATCCTGGCGGCGTTCCGTTAC
GAGTCGCTGCCGGCGACGCTGCACAGCTCGCCGCGCAATCCCCGCATCCCGTGGGAGAAC
CTGCCGGTGCAGGTGGTCGATCGCCTGACCCCCTGGCCTCGGCGCGCAGAGGGCCCCCCG
CGCCGTGCCGGCGTGTCGTCGTTCGGGATCAGCGGGACGAACGCGCATGTCATCCTCGAA
GAAGCGCCGGCCGAGGCGCGGCGCGAGCCTGTCGAGGCCGAGGCGGCGCCTGCGCTATTG
CCGCTGGTGCTGTCGGGTCGAGACGAGGCCGCGGTGAATGCGCAGGCGGGGCGGTGGGCG
AAGTGGCTGGAAGAGCACGGGGAGGTGGGGTGGTCGGACGTGGTGCGCACGGCGGCGCTG
CACCGGACGCACTTCGAGTCGCGGGCGTCGGTGCTTGCGGCGAGCGCTGCGGGAGCTGTG
GAGGGTCTTCGCGCGCTGTCGTCGGGGCGGCCGGATGCCGCGGTGGTGAGCGGGACGGCG
AAGCGAGGCGGGAAGCTTGCGGTGCTGTTCACGGGGCAGGGCAGCCAGCGGCTCGGGATG
GGGAAGAGGCTTTACGAAGTGTACCCCGTGTTCCGTGCGGCGTTCGACGAGGTGTGCCAG
GCGCTGGACGCGCATCTCGACCGTGGGTTGAGAGAGGTGGTGTTCGCGGCCGCGGGCAGC
GAGGAAGGAGCGCAGCTGGAGCGGACGGAGTACACGCAGCCCGGGCTGTTTGCGCTGGAA
GTGGCGCTGTACCGTCAGTGGGAGTCGTGGGGGCTGAAGCCCGCTGCGCTGCTGGGGCAC
TCGATAGGAGAGCTGAGCGCTGCGCACGTGGCGGGTGTGCTGACCCTTGCGGACGCAGCG
AAGCTAGTGTGCGCCCGCGGTCGGCTGATGCAGGGGTGCGAGCCCGGGGGAGCGATGGTG
TCGGTGGAGGCCTCGGAGCCGGAGGTGCAGCGGGCGCTGTCGGAGGTCGGGGCGCAGGGG
CGACTGAGCATCGCCGGGCTGAACGCGCCGATGCAGACGGTGCTGAGCGGGGACGAAGCG
GCGGTGCTCGCGGTGGCGCGACGGCTGGAGGCGCAGGGCCGGCGCACGCGGCGTCTGCGT
GTGTCGCACGCGTTCCACAGCGCGCACATGGACGGGATGCTGGAGGAGTTCGGGAAGGTG
GCGCGGGAGTGCACGTACGCGCGGCCGCAGCTGGCGGTGGTGAGCGGCGTGACGGGCGAG
CTCGGTGGCGAAGAAGCGCTGATGTCGGCCGAGTACTGGGTGAGGCAGGTGCGCGAGGCG
GTGCGCTTCCTGGACGGGATGCGCACGCTTGCGGCGGCGGGGGTGAGCACATACGTCGAG
TGTGGGCCGGATGGCGTGCTGTGCGCGCTGGGGGCGGGGTGCCTGCCGGAGGGAGCCGAG
GCGACGTTTGTGGCGAGCCTGCGGCGAGAGCAGGAGGAAGAGCGCGCGCTGGCGACGGCG
GTGGCGACAGTGCACGTGCAGGGGCACGAGGTGGACTGGGCCCAGGTGCTGTCGGGCCGT
GGCGGCCGGCCCGTGGAGCTGCCGACGTACGCGTTCCAGCGGCAGCGCTACTGGCTGGAA
GCGCCGAAGGCGCGTACCGACGTGGGCTCGGCGGGCTTCAGGGAGTCGGGGCATCCGCTG
CTCGGAGCGGCAACGAAGCTGGCCGACGGCGACGGCCATCTATTCACAGGCCGGCTGTCG
CTGGGCGAGCAGCCGTGGCTTCGCGACCATGCGGTGTTTGGCGAGGTGGTCTTCCCGGGC
ACGGGGATGCTGGACCTCGCGCTGGCGGCTGGGCGCACGGTGGGCAGCGGGGCGCTGTCG
GAGCTCACGATCTCCGAGCCGCTGATGCTCGCGGAGGACGTGGCCGTGCGGCTGCAGCTC
TCGGTCGGGGCGCCCGACGCCGCGGGGCGGCGTGCGTTTGGGCTGTACAGCCAGCCGGAG
CAGGGACCGGGAGATGCCCCCTGGGTGCAGCACGCGACGGGCGTGTTGACCGACGAGACC
CTCGCCACCTCCGGCGAGCTCGATGAGCTGACGACGTGGCCAGTGCCCGGCGCCGAGGCG
GTGGACCTCTCCGGGTTCTACGAGCGGCTGCATGAGCGTGGACTCCGCTACGGCCCGGCC
TTCCAGGGGCTCGTGGAGCTGTCGCGTCGAGACGCGACCTTCTTCGGCCGGGTGGTGTTG
CCCAAAGACGCGACCGACAGCGCCGAGGACTACGGGGTCCATCCGGCGCTGATGGACGCC
GCGCTGCATACGATGGTCGCAGCGTTTGCGGAGGTATCAGCGCCGGACGACGTGCTGCTG
CCTTTCTCGTGGTCGGACGTGGCCTTGCACGCCACGGGGGCGAGCGAGCTCCCGCTCAGG
CTGGAGCTCCCACGAGCCACAGACTCCGCACAGCCAGCCGCCTCGCTGCGCGTTACAGAT
GCCGCCGGCCAGCCGGTGGTGAGCGTCGGTGCCCTCCATCTGCGCCGGGCGACGGCCGAG
CAGCTGCGGGCAGCGACGCATGCCGAGGCGCAGCACCTGTACCGGGTGGACTTCCAGCTC
GTGAGCCTCGTGGAGGCGGGCTCGAAGGTCGACTCCCTGGTGGTGCTCCGTGCGCCTGAG
GGGCGAGGGCGACTGGGCGPAGCGCTGGGTGTGGAGGCGATCGCAGGCCTCGATGCATTG
CTCGCGCGGATCGAGCAGGGAACCCGATTGCCTGAGCGGGTGCTGGTCGACATGACGGCT
GGCAGCTCACAGCGCTCGGACATGGTGATATCGTCGCACGAGGCGACGGGACAGGCGCTG
TCGCTGCTGCAAGCGTGGCTGTCGGAGCCCCGGCTCGAGGGGGTGGAGCTGGTGTGGGTG
ACGCGAGATGCGGTCAGCGCCGCTCCGGGCGACGGTGTCCAAGACCTGGCGCACGCGCCG
CTGTGGGGGCTTGTTCGCACCGCGCGAAGCGAGCACCCCGAGCGCCGGCTGCGCCTGATC
GACGTTGGGACCGAGCCTCTGGACGGCGGGCTGCTGGCGCGCGCGCTGGCGACGGCGACG
GAGCCGGAGCTTGCGCTGCGTGGCGGCGCGGCGATGGCGGCGCGCCTGGTGCGCGTGCCG
GCGGCAGCCGAAGGGCTCACGCCGGCGCGCGGGCTGGACCCGACGGGCACGGTCCTGGTG
ACCGGAGGAACAGGCGAGCTGGGTCAGGCCGTCGCGGAGCATCTGGTGCGCCCACACGGG
GTGCGGCACCTCGTGCTGACGTCGCGCCGTGGGCTGGAGGCGCCCGGGGCCCCAGGCTTC
GTGCAGGCGCTGGAGAAGCTCCGTGCCGAGACCGTGACGGTGGCGGCGTGTGACGTGTCG
AAGCGGGAGGAGGTCGCGCGCGTGCTGGCCGGCATCGAGGCCGCACATCCGCTGACCGCG
GTGCTGCACCTGGCCGGCGTGCTCGACGATGGCGTCATCACCGCGCAGACGCCCGAGCGT
CTCTCGCGGGTGCTGGCGCCGAAGGTGAACGGGGCGCTGCACCTGCACGAGCTGACAGAG
GATCTCGATCTCTCGGCCTTCGTGCTGTTCTCCTCGATGTCCGGGACGCTCGGGACGGCG
GGCCAGAGCAACTACGCGGCGGCCAACAGCTTCCTCGACGCGTTCGCGGCGCATCGCCGC
AGCCGCGGGCTCGCGGCGACGAGCCTGGCGTGGGCCTTCTGGGCGCAAACGGGCGTGGGC
ATGACAGCGCACCTGGGCGAGGCGGAGCTCTCACGTATCCAGCGCGCCGGACTTGTGCCG
ATACGGGTCGAGGAGGGCCTTTCGCTGCTGGACGCCGCGCTTCTGCGCCCCGAAGCGAGC
CTGGTGCCTGCGCACCTCGATCTTGCGCAGATGCAGCGGGGGCTGGAGGCCAGCGGCGAG
CTGCCCGCGCTGCTTCGCGCGCTGCTGCGCCCTGGGCTGCGCAAGGCGTCATCCGCCACG
AGGAAGGAAGCCTCGGCGCTCCGCGAGCGCCTCTCGGAGCTGCCGGAGGCGGAGCGCCTG
AGCTCGCTCGTCGAGCTGGTTCGGGCCGAGGTGGCCGCGGTGCTCGGGCTGCCGCGCAGC
GAGGCCGTTGCGGTAGATCAGGTGCTGAAGGACCTAGGGCTAGATTCGTTGATGGCGGTG
GAGCTGCGCAGTCGGCTCAGCGCCCGAGCCGAGATCCCCCTCCCGGCGACGCTGGTGTTC
GACTACCCGACGCCGCGCGCCGTCGCAGAGCTGCTCCTGAGACAGGCTTTCTCGAAGCAG
CAGGTGACGGCAGCGCGGGCGCGTCGCCGGACGAAGGAAGACGAGGCGATCGCGATCGTA
TCGATGGCGTGCCGGTTGCCAGGGGGCGTGGCGACGCCGGAAGACTACTGGCGTCTCCTG
GCGGAAGGGAAGGACGCCATCGAGCGCTTTCCCTCCCGTTATGACGCGTTCTCTGTTTAT
GACCCCGATCCCGAGGCGGTGGGCAAGAGCTACGTGCGCGAGGGTGGATTCCTGCGGGAT
ATCGATGTCTTCGACGCAGGCTTCTTCGGGATCTCGCCGCGCGAGGCGCAGGCGATGGAT
CCCCAGCAGCGGCTGGTGCTGGAGACGGCGTGGGAGGCGCTGGAGCGAGCCGGCGTGCGG
CCCTCGATGCTGAGCGAGAGCGCCACCGGGGTATACCTGGGCTGGATGGGCTCGGACTAC
GGTGCTCTTCTCGGCAATGACCTCGCCGCGCTGGACGGGTACCAGGGTACGGGGAGCGCG
GCGAGCGTGCTTTCAGGCCGGGTGGCTTACGTGCTGGGGCTTCAGGGCCCAGCGATCACG
GTGGACACGGCGTGCTCGTCGTCGCTGGTGTCGCTGCACCTGGCGTGCACGGCGCTGCGC
CAGGGCGAATGCGACCTGGCGCTGACCGGCGGGGTGATGGTGATGACCACGCCCGCGGGA
TTCGTTGAGTTCAGTCGTGCCCGGGGGCTTGCGCGAGACGGTCGGTGCAAGAGCTTCTCT
GCCCAGGCTGACGGCGTCATCTGGTCCGAAGGGTGCGGGATGCTGTTGCTGAAGCGGCTG
TCTGACGCGCGGCGCGACGGCGACCGTGTGCTGGGGGTGATCCGTGGCTCTGCGGTGAAC
CAGGACGGTCGCAGCCAGGGTCTGACGGCGCCGAACGGCCCTGCCCAGCAGCGGGTGATC
CGGCAGGCGCTGTCGTCGTGTGGTCTGTCGCCCGAGGACATCGACGCGGTCGAGGCGCAT
GGGACGGGTACGAGCCTCGGAGACCCGATCGAGGCCGGAGCGCTGGCGGAGGTGTTTGGA
CCGGAGCGTAGCCCCGAGCGTCCGCTGTACCTGGGGTCGTCGAAGTCGAACCTGGGACAT
GCGCAGGCGGCCGCGGQTGTGGCGGGCGTGATCAAGATGGTGCTGGCGCTGCAGCACGAG
GTGCTGCCGAAGACGCTGCATGCGGAGCAGCCGAGCCCGCACATCGCGTGGGAGGGGAGC
GGGCTGTCATTGCTGCAAGAGGCGCGTCCGTGGCGGCGCAACGGCCGGGTCCGTCGTGCC
GGCGTGTCGTCGTTCGGGATCAGCGGGACGAACGCGCATATCATCCTCGAAGAAGCGCCG
GCCGAGGCGCGGCGCGAGCCTGTCGAGGCCGAGGCGGCGCCTGCGCTATTGCCGCTGGTG
CTGTCGGGTCGAGACGAGGCCTCGGTGGCGGCGCAGGCGGGGCGGTGGGCGAAGTGGCTG
GAAGAGCACGGGGAGGTGGGGTGGTCGGACGTGGTGCGCACGGCGGCGCTGCACCGGACG
CACTTCGAGTCGCGGGCGTCGATGCTTGCGGCGAGCGTGTCCGAGGTGGTGGAGGTGCTG
CGGGCGCTGTCAGAGGGTCGCGGCCACCGGGCGGTGTCCGTGGGCACGGCGCGTGCGCGA
GGCAAGGTGGTGTTCGTGTTCCCCGGCCAAGGGAGCCAGTGGCCGGGGATGGGCCGGGCG
CTGCTGGAGCAGAGCGCAGCGTTCGCGGAGGCGGTGCAGGCGTGCGATGAGGCGCTGCGG
CCGTGGACGGGCTGGTCTGTGCTGTCGGTGCTGCGCGGAGATGGCGGGGAGGAGCAGCCG
TCGCTGGAGCGGGTGGACGTGGTGCAGCCTGCGCTGTTCGCGATGTGCGTGGGTCTGGCC
GCGGCGTGGCGGTCGCTGGGGCTGGAGCCTGCGGCGGTGGTGGGCCACAGCCAGGGCGAG
GTGTCGGCGGCGGTGGTGTGCGGGGCGCTGTCGCTTGCGGAGGGAGCGCGGGTAGTGGCG
CTGCGCAGCCAGGCGGTGCGGCAGCAGTCGGGGATGGGGGCGATGATGCTGGTCGAGCAG
CCGGTGTCGGAGGTGCAGGAGCGCATCGCGCCGTACGGGGAGGCGCTTGCGATAGCGGCG
GTGAACACGTCGAACTCGACGGTGGTGTCGGGTGACGTGGAGGCGGTGGACGGGCTGATG
GTGGAGCTGACGGCAGAAGGTGTGTTCTGCCGGAAGGTGAACGTCGACTACGCGTCGCAC
AGCGCGCACATGGATGCGCTGCTGCCCGAGCTAGGAGCGAAGCTGTCGTCGCTCAGGCCG
AAGGCGACGCAGCTGCCGTTTTACTCGACGGTGACAGGAGAGGTGTCGCGGGGCGAGGCG
CTGGACGGCGAGTACTGGTGCCGCAACCTTCGGCAGACGGTGCGCCTGGACCGAGCGCTG
TCGAAGCTGCTGGAGGACGGGCACGGTGTGTTCGTGGAGGTGAGCGCGCACCCGGTGCTG
GCGATCCCGCTGACGACGGCGTGCGGGGAGGCGCAGGGGGTGGTGGTGGGGAGCTTGCAG
CGCGACGAAGGTGGGTTGTCGCAGCTGTACAGGACGCTGGGGCAGCTGCACGTGCAGGGG
CACGAGGTGGACTGGGCACGGGTGCTGTCCGGCCATGGTGGTCGTGCCGTGGAGCTGCCG
ACGTACGCGTTCCAGCGGCAGCGCTACTGGCTGGATATCTCGAAGGCGCGTAGCGACGTG
AGCTCGGCGGGGCTGAAGGCGGCCGCCCATCCGCTGCTGGGAGCAGCAACGAAGCTGGCT
GAGGGCGATGGCCATCTGTTCACCGGCCGGCTGTCGCTGGGCGAGCATGCGTGGCTCCGC
GACCATGAGGTGTTTGGTAACGTGGTGTTCCCCGGCGCGGGGATGCTGGAGCTTGCGCTG
GCGGCTGGGCGCACGGTGGGCAGCGGGGCGCTGTCGGAGATGGTCTTGGCCGAGCCGCTG
GTGCTCGCCGAGGACGTGGCCGTGCGACTGCACCTGTCGGTCGGCGCCCCOGACGCGGCG
GGCCGGCGTGAGTTTGGGCTGTACAGCCAGCGGGAGCAGGGGCCAGAAGACGCCCCGTGG
GTGCAGCACGCGACGGGGGTGTTGACGGACGAGCCCCGCGGCATCCCTGGTGAGCTCGAT
GAGCTCGCGACGTGGCCAGTGCCCGGCACGGAGGCGGTGGAGCTCTCCGGGTTCTACGAG
CGGCTGCGTGAGCGTGGACTCCACTACGGCCCGGCCTTCCAGGGGCTCGTGGAGCTGTGG
CGTCGAGGCGCGGCGTACTACGGCCGGGTGGCGTTGCCCAAGGCCGCGGGGGACAACGCC
GAGGACTACGGCGTGCATCCGGCGCTGATGGACGCCGCGCTGCACACGATGGTCGCAGCC
TTCTCGGAGATGGCAGAGCAAGGCGCCGTCTTGTTGCCGTTCTCCTGGTCGGATGTGGTG
TTGTACGCCGTGGGCGCGAGCGAGCTCCGTGTCCGGATGGAGCTCCGGGAACAAGGCGCA
CAGCAGGAGGTCACTTCGCTGCACGTCGCTGACCCGACCGGCCAGCTGGTGGCGAGCGTC
GGCGCCTTGCATCTGCGCCGGGCGACGGCCGAGCAGATGCGGACAGCCATCCATGCTGGT
GTCCAGCATCTGTACCGGGTGGACTTCCAGCCTGCGGAGCTCACGGCGTCGGTCGCGGAG
ACGGGCTCGCTGGCGGTGCTCGGTGCGCCGGAGGGGGGAGGACGACTGGCCGAGGCTCTG
GGGGCGGAGGTGGTTGCAGGCCTCCACTCGCTGGTCGCACTGATCGAGCAGGGTGCCCGA
CGGCCTGTGCGGGTGCTGGTCGATGCCACTGCTGCAAATGCCGATCGATCGCCAGTAGCG
GCGTCGCACGAGGCGGCGCGAGAGGCGCTGTCGCTGCTGCAAGCGTGGCTGTCGGAGCCC
CGGCTCGAGGGGGTGCAGCTGGTGTGGGTGACGCGGGATGCGGTCAGCGCTGGTCCGGGC
GACGGTGTCCAGGACCTGGCACACGCACCGCTGTGGGGGCTTGTTCGCACGGCGCGAAGC
GAGCACCCAGAACGCGGCCTGCGCTTGATCGATGTCGGGACCGAGCCTGTGGACGGCGAG
CTGCTGGCGCGCGCGCTGGCGACGGCGACGGCGCCGGAGCTTGCGCTGCGCGGCGGCGCT
GCGCTGGCCGCGCGCCTGGTGCGCGTGCCGGCGGCAGCGGAGACGCTTACGCCGGCTCGC
GGGCTGGACCCGGCAGGCACGGTCCTGGTGACCGGGGGAACAGGCGAGCTGGGTCAGGCC
GTTGCGGAGCATCTGGTGCGCGCGCACGGAGTGCGGCACCTTGTGCTGACGTCGCGCCGT
GGGCTGGAGGCGCCCGGGGCCCGGGAGCTCGTGCAATCGCTGGAGAAGCTCGGCGCCGAG
ACCGTGACGGTGGCGGCGTGTGACGTGTCGAAGCGAGAGGAGGTCGCGCAGGTGCTGGCC
GGAATCGAGGCCGCGCACCCGCTGACCGCGGTGCTGCACCTAGCCGGCGTGCTCGACGAC
GGCGTCCTGTCGTCGCAGACGCCGGAGCGCATTTCACGGGTGTTCGCACCGAAGGTGGAC
GGGGCGCTGCACCTGCACGAGCTGACGCGGGAGCTCGACCTCTCGGCGTTCGTGCTGTTC
TCGTCGGCGGCCGGGACGCTCGGGACGTCGGGCCAGAGCAACTACGCGGCGGCCAACAGC
TTCCTTGACGCGCTCGCGGCGCACCGCCGCAGCCGCGGGCTCGCGGCGACGAGCCTGGCG
TGGGGCTTCTGGGCGCAAGCGGGCGTGGGCATGACAGCGCACCTGGGCGAGGCAGAGCTG
TCGCGCATGCGGCGCAGCGGGGTTGTGCCGATGTCCGTGAGAGAAGGGCTGTCGCTGCTG
GACACCGCGCTCCTGCGCTCCGAGGCGACCCTGGTGCCTTTGCACCTCGATGTTGCGCAG
CTACAGCGGGGGCTGGAGGCCAGCGGCGAGCTGCCGGCGTTGTTCAGGTCCCTGCTGCGC
CCGGGCCTGCGCAAGGCGTCCTCCGCGACGAAGAAAGAGGCGTCTGCGCTCCGCGAGCGC
CTCTCGGAGCTGCCGGAGGCGGAGCGCCTGAGCTCGCTCGTCGAGCTGGTTCGGGCCGAG
GTCGCCGCGGTAATCGGGCTGCCGCGCAGCGAGGGTGTTGAGGCAGACCAGGTGCTGAAG
GACCTGGGGCTCGACTCGCTGATGGCGGTGGCGCTGCGCAACCGCCTCACGTCCCGTACC
GAGACGTCCTTGCCGGCGACGCTGGTCTTTCACTACCCGACGCCGCGCGCCATCGCGAGG
CTGCTGGACAGCCGCCTGCTCTCGGCCCTCTCCCCCCAGGAGGACGGCCCTCCCGCGGAC
CCGGACGGCATGTTGAAATGGGTGCTCAAGCGGGTCTCCGCGAGCCAGATGCAGCAAGCT
GGTGTGCTCCAGCGGCTCCTCCAGCTCGCCGAGCCGAAGCTCCCCCGAACGAACGGTGGC
CACCACGAGAATGGTAGCCGCCAAGAAGAAGAGGATGTCCCGCTCCCGCTGACCATGCAG
GACATCGACAGTAAGCTCGACGCAATTTTGGGTGGTGAGTGA
TmbB
MNSSAASPTLREALTPALKELQRLQASHSDLRSGPIAIVSMACRLPGGVATPEDYWRLLE
EGRDAIEAFPARWDAPSIYDPDPEAVGKTYVREGGFLRDIDLFDAGFFGISPREAQAMDP
QQRLVLETAWEALERAGVRPSALSESSTGVYLGSMGSDYGALYGSDLAALDGYRGTGSAA
SVLSGRVAYVLGLQGPAITVDTACSSSLVSLHLACTALRQGECDLALTGGVMVMTTPAGF
VEFSRLKALARDGRCKSFSARADGVIWSEGCGMLVLKRLSDARRDGDRVLAVIRGSAVNQ
DGRSQGLTAPNGPAQQRVIQQALSSCRLSPEDIDAVEAHGTGTNLGDPIEAGALVEVFGP
GRKAERPLYLGSSKSNLGHAGPAAGVAGVLKMVLSMQHEVLPRTLHAEQPSPHIGWEGSG
LSLLQEARPWRPNGPARPAGVSSPGISGTNAHVILEEAPVEAAREPVEAMREPTATEGVA
MPLLLSGRDEASVGAQAERWAKWLGEHGEVQWSDVVRTAALHRTHFASRASVLAASVSEA
EEALRALSQGRGHRAVSAGTARARGKVVFVFPGQGSQWPGMGRALLEQSAAFAEAVQACD
EALRPWTGWSVLSVLRGDGGEEQPSLERVDVVQPALFAMCVGLAAAWRSLGLEPAAVVGH
SQGEVSAAVVCGALSLAEGARVVALRSQAVRQRSGMGAMMLVERPVSEVQERIAPYGEAL
AIAAVNTSSSTVVSGDVEAVDGIJMVELTAEGVFCRVNVDYASHSAHMDALLPELGAKLS
SLRPKATQLPFYSTVTGEVSRGEALDGEYWCPNLRRTVRLDRALSKLLEDGHGVFVEVSA
HPVLAMPLTTACGEAQGVVVGSLQRDEGGLSQLYRTLGQLHVQGHEVDWTRVLSGHGGRV
VELPTYAFORORYWLDISKARSDVSSAGLKAAAHPLLGAATRLADGEGHLFTGRLSLEAH
PWLRDHEVFGQVVLPGTGTLELVLAAGRAVGSRSLSELTLAEPLVLAEGAARLQVMIGAP
DAAGRREVGLYSQPEQAPEDAPWVQHATGVLTDEPPGIPVELDELSTWPVPGAEEVDLSG
LYERLRERGLHYGPAFQGLVELSRQGTTYFGRVVLPGTEKDRAEAYGVHPALMDAALHTM
VAAFSESPGANEVLVPFAWSDVALHATGASELRVRVELQDGGAHQDTASLQVADSTGQAV
ASIGALHLRRATAEQLRTAVHAGGQHMYQVSFQPVELAAAPLEAGSLVVVGAAEGRGRLA
EALRAEAIADLEALVARLEQGASAPARVAVDTTALGQSQSGVASLSHEATRQALSLLQAW
LSEPRLDAVELVWVTRGAVGAAPDDAVQDLARAPLWGLVRAARSEHPERRLRLIDVGTEP
VDAGLLARALATAAEPELALRGGAALAARLVRAQAAAEELTRGARELDPAGTVLVTGGTG
ELGQAIAAHLVRAHGVRHLVLTSRRGLEAPGARELVQSLEELGAETVTVAACDVSKREEV
ARVLAGIDAARPLSAVLHLAGVLDDGVLTAQTAERLSRVLAPKVDGALHLHELTRELDLA
AFVLFSSAAGTFGAAGQSNYAAANTFLDALAAHRRGGGLAATSLAWGFWTQAGVGMTAHL
GEAELSRMRRNGFVPMPVEEGLALLDAALSRPEASLVPVHLDLAQLQRGLESSGELPALF
RALLRPSLRKASSATRRDASALRERLSALPEAERLNALVELVRGEVAAVAGLQRGEAVAA
DQVLKELGLDSLMAVALRNRLTSRTETSLPATLVFDYPTPPATAELLLKQAFSGLQVKEA
RARVRRRAGKDEPIATVSMACRLPGGVATPDDYWRLLAEGKDAIEGLPARWDGPEVYDPD
PEAAGKSYAREGGFVRDIDLFDANFFGISPREAQSMDPQHRLVLETAWEALERAGVRPSA
LSGSATGVYLGSMGSDYGALHTVDLKELDGYRGTGSAASILSGRVAYALGLQGPAMTVDT
ACSSSLVSLHLACTALRQGECDTAIAGGVTVMSTPALFVEFSRLKGMSRDGRCKSFSVQA
DGAGWAEGCGMLLLKRLSDAQRDGDRVLGVIRGSAVNQDGRSQGLTAPNGPAQQRVIRQA
LSSCGLSPEDIDAVEAHGTGTSLGDPIEAGALAEVFGPERSPERPLYLGSSKSNLGHAQA
AAGVAGVIKMVLSMQHEVLPKTLHAEQPSPHIGWEGSGLSLLQEARPWRRNGRVRRAGVS
SFGISGTNAHIILEEAPAEARREPVEAEAAPALLPLVLSGRDEAAVNAQAGRWAKWLEEH
GEVGWSDVVRTAALHRTHFESRASVLAASAAGAVEGLRALSSGRPDAAVVSGTAKRGGKL
AVLFTGQGSQRLGMGKRLYEVYPVFRAAFDEVCEALDAYLDRGLREVVFAAAGSEEGALL
ERTEYTQPGLFALEVALYRQWESWGLKPAALLGHSIGELSAAHVAGVLSLADAAKLVCAR
GRLMQGCEAGGAMVSVEASEPEVQRALSEVGAQGRLSIAGLNAPMQTVLSGDEAAVLAVA
RRLEAQGRRTRRLRVSHAFHSAHMDGMLEEFGKVARECTYARPRLAVTSGVTGELGGEEA
LMSAEYWVRQVREAVRFLDGMRTLAAAGVSTYVECGPD0VLCALGAGCLPEGAEATFVTS
LRREQEEEPALATAVATVHVQGHEVDWARVLSGRGGRPVELPTYAFQRQRYWLEAPKSAA
TEVNVSSAEQALWNAALEGEGDGVAELLELPDDVPASVGPLLPYLAAWRQRKQAEAAAAS
WLYEEAWQNRPRRVTGSPDVRGTWLVVSPPLAGELAEVVRGALGAAGAEVIVHIAAVERA
QLAAWLREQARLRKEEGELRGVIALTASGEEGALEQGQAPRSLYQTLAVVQALGDAGIGA
RLWLLTQGAVSTEPSEAVVHPLQALTWGLGPALGLEHPBRWGGLLDVPAELDAGVMQHVL
TTLVSDDNEDQVAVRRGGRLVRRTVRVRGEGDGEGWKPRGTVLITGGVGGLGGHLARWIA
GRGAEHLVLASRPGASAPGASELRDELVARGIRVTLAACDVSERAQLAALLAELEQDEAP
LRAVAHLAGIGRRVPLRELEPEQLEQELAAKVKGAWHLHQLLGKRELDAFVLYGSTAGLW
GSGAQAGYGAANAGLDALARYRRARGQAATVLHWGPWAGEGMVTSELESQLRIRGVAVMS
PDKALAGLEMALRLGRTSVAIADVDWSRFAPSFSAARPRPLLDGIEEARRAQESRGPQPA
AGGTALRDTLLGLSEAERRERVRQLVASETAAVLGMTDPSRLDPDRGFLDLGLDSLMAVE
LSKRLQKRTGMTVPSTLSFDHPTQSDVARWLLEQLTPQPRPEPAVREVSREEGWSTPIAI
VGVGLRMPGGASDLESFWQVLVEERDTLRPIPAQRFDVEALYDPDPDAKGKTYVRNASLL
DDVASFDPGFFGISPREAEPMDPQHRLLLETAWSALEDAGVRPERLKGSDTGVFVGVAPS
EYASYRGKSANEDAYALTGTALSFAAGRVAYHLGLQGPAVSTDTACSSSLVAVHLACDAL
RRGDCEVALAAGVQVLANPAGFVLLSRTPALSPDGRCKAFSQAADGyGRGEGVGVLVLMR
LSEAQQQGKRVLGVVRGTAVNQDGASSGITAPNGTAQQKVVRAALRNAGLEPASIDVVEC
HGTGTSLGDPIEVQALGAVYGQGRDMARPLQLGAVKSNIGHLESAAGIAGVCKILAAFRY
ESLPATLHSSPRNPRIPWENLPVQVVDRLTPWPRPAEGPPRRAGVSSFGISGTNAHVILE
EAPAEARREPVEAEAAPALLPLVLSGRDEAAVNAQAGRWAKWLEEHGEVGWSDVVRTAAL
HRTHFESRASVLAASAAGAVEGLRALSSGRPDAAVVSGTAKRGGKLAVLFTGQGSQRLGM
GKRLYEVYPVFRAAFDEVCEALDAHLDRGLREVVFAAAGSEEGAQLERTEYTQPGLFALE
VALYRQWESWGLKPAALLGHSIGELSAAHVAGVLSLADAAKLVCARGRLMQGCEAGGAMV
SVEASEPEVQPALSEVGAQGRLSIAGLNAPMQTVLSGDEAAVLAVARRLEAQGRRTRRLR
VSHAFHSAHMDGMLEEFGKVARECTYARPQLAVVSGVTGELGGEEALMSAEYWVRQVREA
VRFLDGMRTLAAAGVSTYVEC0PDGVLCALGAGCLPEGAEATFVASLRREQEEERALATA
VATVHVQGHEVDWAQVLSGRGGRPVELPTYAFQRQRYWLEAPKARTDVGSAGLRESGHPL
LGAATKLADGDGHLFTGRLSLGEQPWLRDHAVFGEVVFPGTGMLDLALAAGRTVGSGALS
ELTISEPLMLAEDVAVRLQLSVGAPDAAGRRAFGLYSQPEQGPGDAPWVQHATGVLTDET
LATSGELDELTTWPVPGAEAVDLSGFYERLHERGLRYGPAFQGLVELSRRDATFFGRVVL
PKDATDSAEDYGVHPALMDAALHTMVAAFAEVSAPDDVLLPFSWSDVALHATGASELRVR
LELAGGRDSAQAASLRVTDAAGQPWSVGALHLRPATAEQLRAATHIAEAQHLYRVTDFQL
VSLVEAGSKVDSLVVLRAPEGRGRLGEALGVEAIAGLDALLARIEQGTRLPERVLVDMTA
GSSQRSDMVISSHEATGQALSLLQAWLSEPRLEGVELVWVTRDAVSAAPGDGVQDLAHAP
LWGLVRTARSEHPERRLRLIDVGTEPLDGGLLARALATATEPELALRGGAAMAARLVRVP
AAAEGLTPARGLDPTGTVLVTGGTGELGQAVAEHLVRAHGVRHLVLTSRRGLEAPGAPGF
VQALEKLGAETVTVAACDVSKREEVARVLAGIEAAHPLTAVLHLAGVLDDGVITAQTPER
LSRVLAPKVNGALHLHELTEDLDLSAFVLFSSMSGTLGTAGQSNYAAANSFLDAFAAHRR
SRGLAATSLAWGFWAQTGVGMTAHLGEAELSRIQRAGLVPIRVEEGLSLLDAALLRPEAS
LVPAHLDLAQMQRGLEASGELPALLRALLRPGLRKASSATRKEASALRERLSELPEAERL
SSLVELVRAEVAAVLGLPRSEAVAVDQVLKDLGLDSLMAVELRSRLSARAEIPLPATLVF
DYPTPRAVAELLLRQAFSKQQVTAARARRRTKEDEAIAIVSMACRLPGGVATPEDYWRLL
AEGKDAIERFPSRYDAFSVYDPDPEAVGKSYVREGGFLRDIDVFDAGFFGISPREAQAMD
PQQRLVLETAWEALERAGVRPSMLSESATGVYLGWMGSDYGALLGNDLAALDGYQGTGSA
ASVLSGRVAYVLGLQGPAITVDTACSSSLVSLHLACTALRQGECDLALTGGVMVMTTPAG
FVEFSPARGLARDGRCKSFSAQADGVIWSEGCGMLLLKRLSDARRDGDRVLGVIRGSAVN
QDGRSQGLTAPNGPAQQRVIRQALSSCGLSPEDIDAVEAHGTGTSLGDPIEAGALAEVFG
PERSPERPLYLGSSKSNLGHAQAAAGVAGVIKMVLALQHEVLPKTLEAEQPSPHIAWEGS
GLSLLQEARPWRRNGRVRRAGVSSFGISGTNAHIILEEAPAEARREPVEAEAAPALLPLV
LSGRDEASVAAQAGRWALWLEEHGEVGWSDVVRTAALHRTHFESRASMLAASVSEVVEVL
RALSEGRGHRAVSVGTARARGKVVFVFPGQGSQWPGMGRALLEQSAAFAEAVQACDEALR
PWTGWSVLSVLRGDGGEEQPSLERVDVVQPALFAMCVGLAAAWRSLGLEPAAVVGHSQGE
VSAAVVCGALSLAEGARVVALRSQAVRQQSGMGAMMLVEQPVSEVQERIAPYGEALAIAA
VNTSNSTVVSGDVEAVDGLMVELTAEGVFCRKVNVDYASHSAHMDALLPELGAKLSSLRP
KATQLPFYSTVTGEVSRGEALDGEYWCPNLRQTVRLDPALSKLLEDGHGVFVEVSAHPVL
AMPLTTACGEAQGVVVGSLQRDEGGLSQLYRTLGQLHVQGHEVDWARVLSGHGGRAVELP
TYAFQRQRYWLDISKARSDVSSAGLKAAAHPLLGAATKLAEGDGHLFTGRLSLGEHAWLR
DHEVFGNVVFPGAGMLELALAAGRTVGSGALSEMVLAEPLVLAEDVAVRLQLSVGAPDAA
GRREFGLYSQREQGPEDAPWVQHATGVLTDEPRGIPGELDELATWPVPGTEAVELSGFYE
RLRERGLHYGPAFQGLVELWRRGAAYYGRVALPKAAGDNAEDYGVHPALMDAALHTMVAA
FSEMAEQGAVLLPFSWSDVVLYAVGASELRVRMELREQGAQQEVTSLHVADPTGQLVASV
GALHLRRATAEQMRTAIHAGVQHLYRVDFQPAELTASVAETGSLAVLGAPEGGGRLAEAL
GAEVVAGLHSLVALIEQGARRPVRVLVDATAANADRSPVAASHEAAREALSLLQAWLSEP
RLEGVELVWVTRDAVSAGPGDGVQDLAHAPLWGLVRTARSEHPERGLRLIDVGTEPVDGE
LLARALATATAPELALRGGAALAARLVRVPAAAETLTPARGLDPAGTVLVTGGTGELGQA
VAEHLVRAHGVRHLVLTSRRGLEAPGARELVQSLEKLGAETVTVAACDVSKREEVAQVLA
GIEAAHPLTAVLHLAGVLDDGVLSSQTPERISRVFAPKVDGALHLHELTRELDLSAFVLF
SSAAGTLGTSGQSNYAAANSFLDALAAHRRSRGLAATSLAWGFWAQAGVGMTAHLGEAEL
SRMRRSGVVPMSVREGLSLLDTALLRSEATLVPLHLDVAQLQRGLEASGELPALFRSLLR
PGLRKASSATKKEASALRERLSELPEAERLSSLVELVRAEVAAVIGLPRSEGVEADQVLK
DLGLDSLMAVALRNRLTSRTETSLPATLVFDYPTPPAIARLLDSRLLSALSPQEDGPPAD
PDGMLKWVLKRVSASQMQQAGVLQRLLQLAEPKLPRTNGGHHENGSRQEEEDVPLPLTMQ
DIDSKLDAILGGE
tmbC bases 52471-63828
ATGACAAAGCCCGATGATACCCGACTCCAGCGCGCCATGGCGGCGATCGCTCTGCTCGAG
CGGCGCATGGCAGAGCTCGAGGCCTCGCGCACCCAGCCCATCGCGATCGTATCGATGGCG
TGCCGGCTGCCAGGGGGCGTGGCGACGCCGGAAGACTACTGGCGTATCCTGGCGGAAGGG
AAGGACGCGATCGATGGCTTCCCGTCCCGATGGGAGTCGTTCTCTGTTTACGACCCCGAT
CCGGACGTCGTGGGCAAGAGCTACGTGCGCGAGGGTGGATTCCTGCGGGATATCGATGTC
TTCGACGCGGGCTTCTTCGGGATCTCGCCTCATGAGGCGCAGGGGATGGACCCCCAGCAG
CGGCTGGTGCTGGAGACGGCGTGGGAGGCGCTGGAGCGGGCCGGCGTGCGCCCCTCGGCG
CTGAGCGAGAGCTCCACCCGAGTGTACCTGGGCTCGATGGGCTCGGACTACGGTGCTCTT
CTCGGCAATGACCTCGCCGCGCTGGACGGGTACCGAGGGACCGGGAGCGCGGCGAGCGTG
CTTTCAGGCCGGGTGGCTTACGTGCTGGGGCTTCAGGGCCCAGCGATCACGGTGGACACG
GCGTGCTCGTCGTCGCTGGTGTCGCTGCACCTGGCGTGCACGGCGCTGCGCCAGGGCGAA
TGCGACCTGGCGCTGGCCGGCGGGGTGACGGTGATGAGCACCCCCGTGCTGTTCGTGGAG
TTCAGCCGCCTCAAGGGGATGGCCCGAGACGGTCGGTGCAAGAGCTTCTCGGCGCGGGCG
GACGGCGCGGGCTGGGCCGAGGGTTGTGGGATGCTGTTGCTGAAGCGGCTGTCTGACGCG
CGGCGCGACGGCGACCGTGTGCTGGGGGTGATCCGTGGCTCTGCGGTGAACCAGGACGGT
CGCAGCCAGGGTCTGACGGCGCCGAACGGCCCTGCCCAGCAGCGGGTGATCCGGCAGGCG
CTGTCGTCGTGCCGGCTGTCGCCCGAGGACATCGACGCGGTGGAGGCGCATGGGACGGGC
ACGAGCCTCGGAGACCCGATCGAGGCCGGAGCGCTGGCGGAGGTGTTCGGCCCGAAGCGT
AGCCCGGAGCGTCCGCTGTACCTGGGGTCGTCGAAGTCGAACCTGGGACATGCGCAGGCG
GCCGCGGGTGTGGCGGGCGTGATCAAGATGGTGCTGGCGCTGCAGCACGAGGTGCTGCCG
CGAACGCTGCATGCGGAGCAGCCGAGCCCGCACATTGGGTGGGAAGGAAGCGGGCTGTCG
CTGCTGCAAGAGGCGCGTCCGTGGCGGCGCAACGGCCGGGTCCGTCGTGCCGGCGTGTCG
TCGTTCGGGATCAGCGGGACGAACGCGCATATCATCCTCGAAGAAGCGCCGCCCGAGGCG
CGGCGCGAGCCTGTCGAGGCCGAGGCGGCGCCTGCGCTATTGCCGCTGGTGCTGTCGGGT
CGAGACGAGGCCTCGGTGGCGGCGCAGGCGGAGCGCTGGGCGAAGTGGCTGGAAGAGCAC
GCGGAGGTGGGGTGGTCGGACGTGGTGCGCACAGCAGCGCTGCACCGGACGCACTTCGCC
TCTCGCGCATCGGTGCAGGCGGCGAGCGTGTCCGAGGCGGTGGAGGTGCTGCGGGCGCTG
TCGCAGGGTCGCGGCCACCGGGCGGTGTCGGCGGGTACGGCGCGTGCGCGAGGCAAGGTG
GTGTTCGTGTTCCCCGGCCAAGGGAGCCAGTGGCCGGGGATGGGCCGGGCGCTGCTGGAG
CAGAGCGCAGCGTTCGCGGAGGCGGTGCAGGCATGCGACGAGGCGCTGCGGCCGTGGACG
GGCTGGTCTGTGCTGTCGGTGCTGCGCGGCGAGGCGGGTGAGGCAGGTGAGGAGCAGCCG
TCGCTGGAGCGGGTGGACGTGGTGCAGCCCGCGCTGTTCGCGATGTGCGTGGGTCTGGCC
GCGGCGTGGCGGTCGCTGGGGCTGGAGCCTGCGGCGGTGGTGGGCCACAGCCAGGGCGAG
GTGTCGGCGGCGGTGGTGTGCGGAGCGCTGTCGCTTGCGGAGGGAGCGCGGGTAGTGGCG
CTGCGCAGCCAGGCGGTGCGGCAGCAGTCGGGGATGGGGGCGATGATGCTGGTCGAGCGG
CCGGTGTCGGAGGTGCAGGAGCACATCGCGCCGTACGGGGAGGCGCTTGCGATACCGGCG
GTGAACACGTCGAGCTCGACGGTGGTGTCGGGTGACGTGGAGGCGGTGGACGGGCTGATG
GTGGAGCTGACGGCAGAAGGTGTGTTCTGCCGGAAGGTGAACGTCGACTACGCGTCGCAC
AGCGCGCACATGGATGCGCTGCTGCCCGAGCTAGGAGCGAAGCTGTCGTCGCTCAGGCCG
AAGGCGACGCAGCTGCCGTTTTACTCGACGGTGACAGGAGAGGTGTCGCGGGGCGAGGCG
CTGGACGGCGAGTACTGGTGCCGCAACCTTCGGCAGACGGTGCGCCTGGACCGAGCGCTG
TCGAAGCTGCTGGAGGACGGGCACGGTGTGTTCGTGGAGGTGAGCGCGCACCCGGTGCTG
GCGATGCCGCTGACGACGGCGTGCGGGGAGGCGCAGGGGGTGGTGGTGGGGAGCCTGCAG
CGCGACGAAGGTGGGTTGTCGCAGCTGTACAGGACGCTGGGGCAGCTGCACGTGCAGGGG
CACGAGGTGGACTGGGCACGGGTGCTGTCGGGCCACGGCGGTGGTGTCGTGGAGCTGCCG
ACGTACGCGTTCCAGCGGCAGCGCTACTGGCTGGATATCTCGAAGGCGCGTAGCGACGTG
AGCTCGGCGGGGCTGAAGGCGGCCGCCCATCCGCTGCTGGGAGCGGCAACGAAGCTGGCT
GAGGGCGATGGCCATCTGTTCACCGGCCGGCTGTCGCTGGGCGAGCATGCGTGGCTCCGC
GACCATGAGGTGTTTGGTAACGTGGTGTTCCCCGGCGCGGGGATGCTGGAGCTTGCGCTG
GCGGCTGGGCGCACGGTGGGCAGCGGGGCGCTGTCGGAGATGGTCTTGGCCGAGCCGCTG
GTGCTCGCCGAGGACGTGGCCGTGCGACTGCAGCTGTCGGTCGGCGCGCC
GGACGCGGCGGGCCGGCGTGAGTTTGGGCTGTACAGCCAGCTGGAGCAGGGGCCAGAAGA
CGCCCCGTGGGTGCAGCACGCGACGGGCGTGTTGGCGGACGAGCCCCGCGGCATCCCTGG
TGAGCTCGATGAGCTCGCGACGTGGCCAGTGCCCGGCGCGGAGGAGGTGGATCTCTCCGG
GTTCTACGAGCGGCTGCGTGAGCGTGGACTCCACTACGGCCCGGCCTTCCAGGGGCTCGT
GGAGCTGTGGCGTCGAGGCACGACGTTGTTCGGCCGCGTGGTGTTGCCCAAGGCCGCGGG
GGACAGCGCCGAGGACTACGGCGTGCATCCGGCGCTGATGGACGCCGCGCTGCATACGAT
GGTTGCGGCCCTCTCCGAGAGGCCAGGAGCGAACGCGGTGCTCCTGCCGTTCGCTTGGTC
GGACGTGGTGTTGCTCGCCATGGGGGCGAGCGAGCTCCGGGTCCATGTGGATCTCCAGGA
TCACGGCGCAGAGCAGGCGATGGCTTCGCTGTACGTCGCTGACTCCGTGGGGCAACTTGT
GGTGAGCATCGGCGAGCTGAAGCTGCGCTGGGCGACGGCCGAGCAGCTGAGGGAGGCGAC
CCGTGCCGAGGCGCAGCATCTGTACCGGGTGAACTTCCGCCCTGTGCGTCTCGTGGACGG
TTCGTCGGAGTCGGCCCCGCCAATGCTTGTCATAGTCTCGGAGGGGCAAGGGCAACTGGC
CGAGATCCTGGAGGCGGAAGCGGTCGCGAGCCTCGATGTACTGCTTGCACGTCTCGCGCA
GGGCGCGAGCGCGCCTGTGCGGGTGCTGGTCGATGCCACTGCTGCAAATGCCGGTCGATC
GCCAGTAGCGGCGTCGCACGAGGCGGCGCAAGAGGCGCTGTCGCTGCTGCAAGCGTGGCT
GTCGGAGCCCCGGCTCGAGGGGGTGGAGCTGGCGTGGGTGACGCGAGATGCGGTCAGCGC
CGCTCCGGGCGACGGTGTCCAGGACCTGGCACACGCGCCGCTGTGGGGGCTTGTTCGCAC
GGCGCGAAGCGAGCACCCCGAGCGCCAACTGCGCCTGATCGACGTCGGGACCGAGCCTGT
GGATGGCGGGCTGCTGGAGCGCGCGCTGGCGACGGCGACGGAGCCGGAGCTTGCGCTGCG
CGGCGGCGCTGCGCTGGCCTCGCGCCTGGTACGCGTGCAGGCGGTGGAGGAGGTCACCCG
AACTCGCGGGCTGGACCCGGCAGGCACGGTCCTGGTGACCGGGGGAACAGGCGAGCTGGG
TCAGGCCGTTGCGGAGCATCTGGTGCGCGCGCACGGAGTGCGGCACCTTGTGCTGACGTC
GCGCCGTGGGCTGGAGGCGCCCGGGGCCCGGGAGCTCGTGCAATCGCTGGAGAAGCTCGG
CGCCGAGACCGTGACCGTGGCGGCGTGTGACGTGTCGAAGCGGGAGGAGGTCGCGCAGGT
GCTGGCCGGAATCGAGGCTGCGCACCCGCTGACCGCGGTGCTGCACCTGGCCGGCGTGCT
CGACGACGGCGTCCTGTCGTCGCAGACGCCGGAGCGCATTTCACGGGTGTTCGCGCCGAA
GGTGGACGGGGCGCTGCACCTGCACGAGCTGACGCGGGAGCTCGACCTCTCGGCGTTCGT
GCTGTTCTCGTCGGCGGCCGGGACGCTCGGGACGTCGGGCCAGAGCAACTACGCGGCGGC
CAACAGCTTCCTCGACGCGCTCGCGGCGCACCGCCGCAGCCGCGGGCTCGCGGCGACGAG
CCTGGCGTGGGGGTCGTGGATGCCGGAGCCGACGGGGACCGCCCGACCGAGCACCGCCGA
GCCGGGAGCCACGAAACAACCTGGTCTGATGCCGATGTCCTTCAGCGCGGGGTTGTCGCT
TCTGGACGCGACGCTCTCGCGGCCTGAAGCGAACCTGGTTCCGGCGTACCTGGATCTCGC
CAGGCTGCAGAGAGGAGTCGAGGCCAGCGGCGAGCTGCCTGCGCTGCTCCGGGCGCTGCT
GCGTCCGGGCCTGCGCAAGGCTGCGTTTGGAGCCCAGGAAGCCTCGGCGCTCCGTGAGCG
TCTCGCGAGGCTGCCGGAGTCGGAGCGCCTGAATGCGCTCGTCGCGCTGGTTCAGGCCGA
GGTCGCGACCGTGATGGGGCTGCCGCGCAGCGAGGCTGTGGCAGCAGATCAGGTGCTCAA
GGAGCTGGGGCTCGACTCGCTGATGGCGGTGGCGCTGCGCAACCGCCTCACCGCCCGGAC
CGAGACGCCATTGCCGGCGACGCTGGTCTTTGACTACCCGACGCCGCGGGCCGTCGCGGA
GCTGCTGCTGAAGCAGGCGTTCTCGGAGCTGAAGAGCGCGGGGGCCCGCCCGCGGGGGCG
TCGTCGCGGGCAGGAGGACGAGCCCATCGCGATCGTGTCGATGGCGTGCCGGCTGCCAGG
GGGCGTGGCGACGCCGGAAGACTACTGGCGTCTCCTGGCGGAAGGGAAGGACGCGATCGA
GCGCTTCCCTGCACGCTGGGACGCCCTAAGTATCTACGCTCCTGATCCGGACGCCGTGGG
CAAGAGCTATGCGCGCGAGGGGGGCTTCGTTCACGGCGTCGACCTCTTCGACGCGGGCTT
TTTCGGGATCTCGCCGCGCGAGGCGCACGCGATGGATCCCCAGCAGCGGCTGGTGCTGGA
GACGGCGTGGGAGGCGCTGGAGCGGGCCGGCGTGCGCCCCTCGGCGCTGAGCGGGAGCGC
CACCGGGGTATACCTGGGCTCGCCCGGCTCGGATTACGGCAGCCAGATCGGGAGCGCCCT
GGATGCTCTGGACGGGTACCAGATGACGGGGAATCTTGGCTCCGTGATCTCTGGCCGGGT
GGCTTACGTGCTGGGGCTTCAGGGCCCAGCGATCACGGTGGACACGGCGTCCTCGTCGTC
GCTGGTGTCGCTGCACCTGGCGTGCACGGCGCTGCGCCAGGGCGAATCCGACCTGGCGCT
GGCCGGCGGGGTGACGGTGATGAGCACCCCCGCGCTGTTCGTGGAGTTCAGCCGTCTCAA
GGGGATGGCCCGAGACGGTCGGTGCAAGAGCTTCTCGGCGCAGGCGGACGGCGCGGGCTG
GGCCGAGGGTTGTGGGATGCTGTTGCTGAAGCGGCTGTCTGACGCGCGGCGCGACGGCGA
CCGTGTGCTGGGGGTGATCCGTGGCTCTGCGGTGAACCAGGACGGTCGCAGCCAGGGTCT
GACGGCGCCGAACGGCCCTGCCCAGCAGCGGGTGATCCGGCAGGCGCTGTCGTCGTGCCG
GCTGTCGCCCGAGGACATCGACGCGATGGAGGCGCACGGGACAGGCACGAGCCTTGGAGA
CCCGATCGAGGCCGGAGCGCTGGCGGAGGTGTTTGGACCGGAGCGTAGCCCCGAGCGTCC
GCTGTACCTGGGTTCGTCGAAGTCGAACCTGGGGCACACGGGACCTGCCGCGGGCGTGGC
GGGCGTGATCAAGATGGTGCTGGCGCTGCAGCGCGAGGTGCTGCCGAAGACGCTGCACGC
GGAGCAGCCGAGCCCGCACATCGCATGGGAGGGGAGCGGGCTGTCATTGCTGCAAGAGGC
GCGTCCGTGGCGGCGCAACGGCCGGGTCCGTCGTGCCGGCGTGTCGTCGTTCGGGATCAG
CGGGACGAACGCGCATATCATCCTCGAAGAAGCGCCGGCCGAGGCGCGGCGTGCGCCCGT
CGAGGTCAAGGCCGAGGTGGCGCCGGCGGCGATGCCGCTGGTGCTGTCGGGTCGAGACGA
GGCTGCGGTGAATGCGCAGGCCGGGCGGTGGGCGAAGTGGCTGGAAGGGCACGCGGAGGT
GGGGTGGTCGGACGTGGTGCGCACGGCAGCGCTGCACCGGACGCACTTCGCCTCTCGCGC
ATCGGTGCAGGCGGCGAGCGTGTCCGAGGCGGTGGAGGTGCTGCGGGCGCTGTGGCAGGG
TCGCGGCCACCGGGCGGTGTCGGCGGGCACGGCGCGTGCGCGAGGCAAGGTGGTGTTCGT
GTTCCCCGGCCAGGGGAGCCAGTGGCCGGGGATGGGCCGAGCGCTGCTGGAGCAGAGCGC
AGCGTTCGCGGAGGCGGTGCAGGCGTGCGATGAGGCGCTGCGGCCGTGGACGGGCTGGTC
TGTGCTGTCGGTGCTGCGCGGAGATGGCGGGGAGGAGCAGCCGTCGCTGGAGCGGGTGGA
CGTGGTGCAGCCCGCGCTGTTCGCGATGTGCGTGGGTCTGGCCGCGGCGTGGCGGTCGCT
GGGGCTGGACCCTGCGGCGGTGGTGGGCCACAGCCAGGGCGAGGTGTCGGCGGCGGTGGT
GTGCGGAGCGCTGTCGCTTGCGGAGGGAGCGCGGGTAGTGGCGCTGCGCAGCCAGGCGGT
GCGGCAGCAGTCGGGGATGGGGGCGATGATGCTGGTCGAGCGGCCGGTGTCGCAGGTGCA
GGAGCACATCGCGCCGTACGGGGAGGCGCTTGCGATAGCGGCGGTGAACACGTCGAGCTC
GACGGTGGTGTCGGGTGACGTGGAGGCGGTGGACGGGCTGATGGTGGAGCTGACGGCAGA
AGGTGTGTTCTGCCGGAAGGTGAACGTCGACTACGCGTCGCACAGCGCGCACATGGATGC
GCTGCTGCCCGAGCTAGGAGCGAAGCTGTCGTCGCTCAGGCCGAAGGCGACGCAGCTGCC
GTTTTACTCGACGGTGACAGGAGAGGTGTCGCGGGGCGAGGCGCTGGACGGCGAGTACTG
GTGCCGCAACCTTCGGCAGACGGTGCGCCTGGACCGACCGCTGTCGAAGCTGCTGGAGGA
CGGGCACGGTGTGTTCGTGGAGGTGAGCGCGCACCCGGTGCTGGCGATGCCGCTGACGAC
GGCGTGCGGGGAGGCGCAGGGGGTGGTGGTGGGGAGCCTGCAGCGCGACGAAGGTGGGTT
GTCGCAGCTGTACAGGACGCTGGGGCAGCTGCACGTGCAGGGGCACGAGGTGGACTGGGC
ACGGGTGCTGTCGGGCCACGGCGGTGGTGTCGTGGAGCTGCCGACGTACGCGTTCCAGCG
GCAGCGCTACTGGCTGGATATCTCGAAGGCGCGTAGCGACGTGAGCTCGGCGGGGCTGAA
GGCGGCCGCCCATCCGCTGCTGGGAGCGGCAACGAAGCTGGCTGAGGGCGATGGCCATCT
GTTCACCGGCCGGCTGTCGCTGGGCGAGCATGCGTGGCTCCGCGACCATGAGGTGTTTGG
TAACGTGGTGTTCCCCGGCGCGGGGATGCTGGAGCTTGCGCTGGCGGCTGGGCGCACGGT
GGGCAGCGGGGCGCTGTCGGAGATGGTCTTGGCCGAGCCGCTGGTGCTCGCCGAGGACGT
GGCCGTGCGACTGCAGCTGTCGGTCGGCGCGCCGGACGCGGCGGGCCGGCGTGAGTTTGG
GCTGTACAGCCAGCTGGAGCAGGGGCCAGAAGACGCCCCGTGGGTGCAGCACGCGACGGG
GGTGTTGACGGACGAGCCCCGCGGCATCCCTGGTGAGCTCGATGAGCTCGCGACGTGGCC
AGTGCCCGGCGCGGAGGCGGTGGATCTCTCCGGGTTCTACGAGCGGCTGCGTGAGCGTGG
ACTCCACTACGGCCCGGCCTTCCAGGGGCTCGTGGAGCTGTGGCGTCGAGGCACGACGTT
GTTCGGCCGCGTGGTGTTGCCCAAGGCCGCGGGGGACAGCGCCGAGGACTACGGCGTGCA
TCCGGCGCTGATGGACGCCGCGCTGCATACGATGGTTCCGGCCCTCTCCGAGAGGCCAGG
AGCGAACGCGGTGCTCCTGCCGTTCGCTTGGTCGGACGTGGTGTTGCTCGCCATGGGGGC
GAGCGAGCTCCGGGTCCGGATGGAGCTCCAGGAGACCGCGGACTCCAGGCAGATCACGGC
TTCGTTGTCCGTCGCAGATGCGATCGGCCAGCCCGCGGCGAGCGTCGGCGAGCTGCAGCT
GCGCTGGGCGACAGCCGAGCAGCTGAGGGCGGCGATCCGTACCGAGGCGCAGCATCTGTA
TCGGGTGGACTTCCGCCCTGTGCGCCTCGTGGCCGGCTCGTCGGAGCCGGCCCGGCCAAC
GCTTGTCATGGTAGCGGAGGGGCAAGGGCCACTGGCCGAGATCCTGGAGGCGGAGGCGGT
GGCGAGCCTCGATATATTGCTCGCACGTCTCGAGCAGGGCGCGAGCGCGCCTGTGCGGGT
GGTGGTCGATGTCACTGCTGCCGCTACCAGCCAGCCGGGCGTGCCGGCGGCGTCGCACGA
GGCGACGCAGGAGGCGCTGTCGTTGCTGCAAGCGTGGCTGTCGGAGCCCCGGCTCGAGGG
GGTGGAGCTGGTGTGGGTGACGCGAGATGCGGTCAGCGCCGCTCCGGGCGACGGTGTCCA
GGACCTGGCACACGCGCCGCTGTGGGGGCTTGTTCGCACGGCGCGAAGCGAGCACCCCGA
GCGCCAACTGCGCCTGATCGACGTCGGGACCGAGCCTGTGGATGGCGAGCTGCTGGCGCG
CGCGCTGGCGACGGCGACGGAGCCGGAGCTTGCGCTGCGCGGCGGCGCTGCGCTGGCCGC
GCGCCTGGTGCGCGTGCCGGCGGCAGCGGAAACGCTTACGCCGGCGCGCGGGCTGGACCG
GACGGGCACGGTCCTGGTGACCGGGGGAACAGGCGAGCTGGGTCAGGCCGTTGCGGAGCA
TCTGGTGCGCGCGCATGGAGTGCGGCACCTTGTGCTGACGTCGCGCCGTGGGCTGGAGGC
GCCCGGGGCCCGGGAGCTCCTGCAATCGCTGGAGAAGCTCGGCGCCGAGACCGTGACGGT
GGCGGCGTGTGACGTGTCGAAGCGAGAGGAGGTCGCGCAGGTGCTGGCCGGCATCGAGGC
CGCGCACCCGCTGACCGCGGTGCTGCACCTGGCCGGCGTGCTCGACGACGGCGTCCTGTC
GTCGCAGACGCCGGAGCGCATTTCACGGGCGTTCGCGCCGAAGGTGGACGGGGCGCTGCA
CCTGCACGAGCTGACGCGGGAGCTCGATCTGTCGGCGTTCGTGTTGTTCTCCTCGATGTC
CGGGACGCTCGGGACGTCGGGCCAGAGCAACTACGCGGCGGCCAACAGCTTCCTCGACGC
GCTCGCGGCGCACCGCCGCGGCTGCGGGCTCGCGGCGACGAGCCTGGCGTGGGGCTTCTG
GGCGCAAGCGGGTGTGGGCATGACAGCGCACCTGGGCGAGGCAGAGCTGTCACGCATCCG
GCGCGCAGGGCTTGTGCCGATGTCGATTGAGGAGGGCCTTTCGCTGCTGGATGCCGCGCT
CCTGCGCGCCGAGGCGAGCCTGGCCCCGGTGCGCTTCGATCTGGCGACGCTGCAGCGCAA
GCTCGACACCGGCGGCGCGCTGCCCCCGCTGTTCCTGGCCTTGCTGCGCCCGGCCCTGCG
CCGGGTTTCCCCGGCATCACAGGGCACCTCGGCGATTCGCGAGCGTCTCGTGGCGCTGCC
TGAGCAGGAGCGGTTGAAGTCGCTCGTCGCTCTGGTTCAAGCCGAGGTCGCCGCGGTGCT
CGGGTTGCAGGGAGCGGCTACCATCCGGGCCGACCAGCCCCTGCTGGAGCTCGGAATGGA
CTCCTTGATGGCGGTGGAGCTTCGAAACCGGCTCTCCAGCCTGATCGGCGCCACGCTCCC
CGTCACCGTGGCCTTCACACATCCAGACTCCAGGTCCATCGGTGCTTTCCTCCTCGACAT
GCTCCCTTCGGCCGGGGAGCGCTCGCTCCCGCGGAATGACAAGGTCGCCGGCAAGTGGCT
TCGCATCCTCAGGTCGCCGCGCGTCCCCGCCGTCCGGATCGTCTGTTTCCCGGGCGCTGG
AGGGGCAGCCTCTCTGTTCTTACCCCTCGCCCAGCACGTGGCCGATCACGTGGAGCTCGT
GGCGATCCAGGCCCCGGCTCGGGGTGATCGGCTCGCAGAGACGTCGGTCACGGACATGAG
CGTTTTCGTCAGCGAGGTGTGCGACGCGCTGCGCGGTCGACTCGACTTGCCCACGCTGTT
CTTCGGCTACAGCTTCGGGACCTGGACAGCTTACGCGGTGCTGTGCGGCGCCTTGTCTCG
TGGCGTGCATCGTGCGCCGCTCGGCCTCGCCGTCGCCTGCATGACCCCGCCGTCCGACGC
ACGACAGCGCACGATGCAACTGGGGATCGAGGAGGACGACGACACCGTCGTGAAGAGAAT
GGTCGCCGTCGGGGCATGGCCGGAGGCAGCCCTGGACGACGGCGAGCTTCGTGCGGCGCT
CCTCCCCTCGTTCCGTGCGGATGCGCGCCTTGGAATGAGCTATCGGTGGGCCGAGGAGAA
GCTGCTGGACGTCCCTGTCCTCGCCGTGGCAGCGACGAGGGACGAGCTATCCCAGATGCG
TCATCCATCGAGGCGTGGCGTCAGGTGA
MTKPDDTRLQRAMAATALLERPMAELEASRTQPIAIVSMACRLPGGVATPEDYWRTLAEG
KDAIDGFPSRWESFSVYDPDPDVVGKSYVREGGFLRDTDVFDAGFFGTSPHEAQGMDPQQ
RLVLETAWEALERAGVRPSALSESSTGVYLGSMGSDYGALLGNDLAALDGYRGTGSAASV
LSGRVAYVLGLQGPAITVDTACSSSLVSLHLACTALRQGECDLALAGGVTVMSTPVLFVE
FSRLKGMARDGRCKSFSARADGAGWAEGCGMLLLKRLSDARRDGDRVLGVTRGSAVNQDG
RSQGLTAPNGPAQQRVIRQALSSCRLSPEDTDAVEAHGTGTSLGDPTEAGALAEVFGPKR
SPERPLYLGSSKSNLGHAQAAAGVAGVIKMVLALQHEVLPRTLHAEQPSPHIGWECSGLS
LLQEARPWRRNGRVRRAGVSSFGISGTNAHIILEEAPAEARREPVEAEAAPALLPLVLSG
RDEASVAAQAERWAKWLEEHAEVGWSDVVRTAALHRTHFASPASVQAASVSEAVEVLRAL
SQGRGHPAVSAGTARARGKVVFVFPGQGSQWPGMGPALLEQSAAFAEAVQACDEALRPWT
GWSVLSVLRGEAGEAGEEQPSLERVDVVQPALFAMCVGLAAAWRSLGLEPAAVVGHSQGE
VSAAVVCGALSLAEGARVVALRSQAVRQQSGMGAMMLVERPVSEVQEHIAPYGEALATAA
VNTSSSTVVSGDVEAVDGLMVELTAEGVFCRKVNVDYASHSAHMDALLPELGAKLSSLRP
KATQLPFYSTVTGEVSRGEALDGEYWCRNLRQTVRLDRALSKLLEDGHGVFVEVSAHPVL
AMPLTTACGEAQGVVVGSLQRDEGGLSQLYRTLGQLHVQGHEVDWARVLSGHGGGVVELP
TYAFQRQRYWLDISKARSDVSSAGLKAAAHPLLGAATKLAEGDGHLFTGRLSLGEHAWLR
DHEVFGNVVFPGAGMLELALAAGRTVGSGALSEMVLAEPLVLAEDVAVRLQLSVGAPDAA
GRREFGLYSQLEQGPEDAPWVQHATGVLADEPRGTPGELDELATWPVPGAEEVDLSGFYE
RLRERGLHYGPAFQGLVELWRRGTTLFGRVVLPKAAGDSAEDYGVHPALMDAALHTMVAA
LSERPGANAVLLPFAWSDVVLLAMGASELRVHVDLQDHGAEQAMASLYVADSVGQLVVSI
GELKLRWATAEQLREATPAEAQHLYRVNFRPVRLVDGSSESAPPMLVIVSEGQGQLAEIL
EAEAVASLDVLLARLAQGASAPVRVLVDATAANAGRSPVAASHEAAQEALSLLQAWLSEP
RLEGVELAWVTRDAVSAAPGDGVQDLAHAPLWGLVRTARSEHPERQLRLIDVGTEPVDGG
LLERALATATEPELALRGGAALASRLVRVQAVEEVTRTRGLDPAGTVLVTGGTGELGQAV
AEHLVPAHGVRHLVLTSRRGLEAPGARELVQSLEKLGAETVTVAACDVSKREEVAQVLAG
IEAAHPLTAVLHLAGVLDDGVLSSQTPERISRVFAPKVDGALHLHELTRELDLSAFVLFS
SAAGTLGTSGQSNYAAANSFLDALAAHRRSRGLAATSLAWGSWMPEPTGTARPSTAEPGA
TKQPGLMPMSFSAGLSLLDATLSRPEANLVPAYLDTARLQRGVEASGELPALLPALLRPG
LRKAAFGAQEASALRERLARLPESERLNALVALVQAEVATVMGLPRSEAVAADQVLKELG
LDSLMAVALRNRLTARTETPLPATLVFDYPTPRAVAELLLKQAFSELKSAGARPRGRRRG
QEDEPIAIVSMACRLPGGVATPEDYWRLLAEGKDAIERFPARWDALSIYAPDPDAVGKSY
AREGGFVHGVDLFDAGFFGISPREAQANDPQQRLVLETAWEALEPAGVRPSALSGSATGV
YLGSAGSDYGSQIGSALDALDGYQMTGNLGSVISGRVAYVLGLQGPATTVDTACSSSLVS
LHLACTALRQGECDLALAGGVTVMSTPALFVEFSRLKGMARDGRCKSFSAQADGAGWAEG
CGMLLLKRLSDARRDGDRVLGVIRGSAVNQDGRSQGLTAPNGPAQQRVIRQALSSCRLSP
EDIDAMEAHGTGTSLGDPIEAGALAEVFGPERSPERPLYLGSSKSNLGHTGPAAGVAGVI
KMVLALQREVLPKTLHAEQPSPHTAWEGSGLSLLQEARPWRRNGRVRRAGVSSFGTSGTN
AHIILEEAPAEARPAPVEVKAEVAPAAMPLVLSGRDEAAVNAQAGRWAKWLEGHAEVGWS
DVVRTAALHRTHFASPASVQAASVSEAVEVLRALWQGRGHRAVSAGTARARGKVVFVFPG
QGSQWPGMGPALLEQSAAFAEAVQACDEALRPWTGWSVLSVLRGDGGEEQPSLERVDVVQ
PALFANCVGLAAAWRSLGLEPAAVVGHSQGEVSAAVVCGALSLAEGARVVALRSQAVRQQ
SGMGANMLVERPVSEVQEHIAPYGEALAIAAVNTSSSTVVSGDVEAVDGLMVELTAEGVF
CRKVNVDYASHSAHMDALLPELGAKLSSLRPKATQLPFYSTVTGEVSRGEALDGEYWCRN
LRQTVRLDPALSKLLEDGHGVFVEVSAHPVLANPLTTACGEAQGVVVGSLQRDEGGLSQL
YRTLGQLHVQGHEVDWARVLSGHGGGVVELPTYAFQRQRYWLDISKARSDVSSAGLKAAA
HPLLGAATKLAEGDGHLFTGRLSLGEHAWLRDHEVFGNVVFPGAGMLELALAAGRTVGSG
ALSEMVLAEPLVLAEDVAVRLQLSVGAPDAAGRREFGLYSQLEQGPEDAPWVQHATGVLT
DEPRGIPGELDELATWPVPGAEAVDLSGFYERLRERGLHYGPAFQGLVELWRRGTTLFGR
VVLPKAAGDSAEDYGVHPALMDAALHTMVAALSERPGANAVLLPFAWSDVVLLAMGASEL
RVRMELQETADSRQITASLSVADAIGQPAASVGELQLRWATAEQLRAATRTEAQHLYRVD
FRPVRLVAGSSEPARPTLVMVAEGQGPLAEILEAEAVASLDILLARLEQGASAPVRVVVD
VTAAATSQPGVAASHEATQEALSLLQAWLSEPRLEGVELVWVTRDAVSAAPGDGVQDLA
HAPLWGLVRTARSEHPERQLRLIDVGTEPVDGELLARALATATEPELALRGGAALAARLV
RVPAAAETLTPARGLDRTGTVLVTGGTGELGQAVAEHLVRAHGVRHLVLTSRRGLEAPGA
RELLQSLEKLGAETVTVAACDVSKREEVAQVLAGTEAAHPLTAVLHLAGVLDDGVLSSQT
PERISPAFAPKVDGALHLHELTRELDLSAFVLFSSMSGTLGTSGQSNYAAANSFLDALAA
HRRGCGLAATSLAWGFWAQAGVGMTAHLGEAELSRIRRAGLVPMSIEEGLSLLDAALLRA
EASLAPVRFDLATLQRKLDTGGALPPLFLALLRPGLRRVSPASQGTSATRERLVALPEQE
RLKSLVALVQAEVAAVLGLQGAATTRADQPLLELGMDSLMAVELRNRLSSLIGATLPVTV
AFTHPDSRSIGAFLLDMLPSAGERSLPRNDKVAGKWLRILRSPRVPAVRIVCFPGAGGAA
SLFLPLAQHVADDVELVAIQAPARGDRLAETSVTDMSVFVSEVCDALRGRLDLPTLFFGY
SFGTWTAYAVLCGALSRGVHRAPLGLAVACMTPPSDARQRTMQLGIEEDDDTVVKRMVAV
GAWPEAALDDGELRAALLPSFRADARLGMSYRWAEEKLLDVPVLAVAATRDELSQMRHPS
RRGVR
orf5a
GTGGGCCGAGGAGAAGCTGCTGGACGTCCCTGTCCTCGCCGTGGCAGCGACGAGGGACGA
GCTATCCCAGATGCGTCATCCATCGAGGCGTGGCGTCAGGTGACGACAGGAGACTTTGTC
ATGAGCCACCTCGACGGCACGCACTCGCTCGTCCTGGATGATCCTGCGAGCCTGGCACGA
GAGCTGTCGACGTGCGTGGAGACGCAGCTGTGGCCAGGGAGAGAGCGCTCGAGCGACCGC
GCCACCCAGGCACCCGGCGACAGCCGCGGGGACGGCGCGGCGGCCGGTGTCGACGGCATG
CCGCCGCTCGGAGAAGAGCGCGAGAGACCGGGCACCAGCCCATCGCAAGCTTACGTGGGC
TGA
VGRGEAAGRPCPRRGSDEGRAIPDASSIEAWRQVTTGDFVMSHLDGTHSLVLDDPASLAR
ELSTCVETQLWPQRERSSDPATQAPGDSRGDGAAAGVDGMPPLGEERERPGTSPSQAYVG
orf5b
GTGCCCGGTCTCTCGCGCTCTTCTCCGAGCCGCGGCATGCCGTCGACACCGGCCGCCGCG
CCGTCCCCGCGGCTGTCGCCGGGTGCCTGGGTGGCGCGGTCGCTCGAGCGCTCTCTCCCT
GGCCACAGCTGCGTCTCCACGCACGTCGACAGCTCTCGTGCCAGGCTCGCAGGATCATCC
AGGACGAGCGAGTGCGTGCCGTCGAGGTGGCTCATGACAAAGTCTCCTGTCGTCACCTGA
VPGLSRSSPSGGMPSTPAAAPSPRLSPGAWVARSLERSLPGHSCVSTHVDSSRARLAGSS
RTSECVPSRWLMTKSPVVT
orf 6
ATGCTGCCAAGGAACGACATCATCCATGCTCATGCCGACGTGAACGGCGTCCGCCTCCAT
TACGCGTCCAGGGGCGCGGGCAAGCTCATCCTGTTCATCCACGGCTTCCCCGAGCTCTGG
TACGCCTGGAAGCGGCAGCTCTTCGATTTCGGCCGCCACCACCGCGCGGTGGCCCTGGAC
CAGCGCGGCTACAACCTGTCGTCGAAGCCGTCGGCCGTGGACGCCTACGGCATCGACCTC
CTGGCCGCGGACATCGCGGCGCTGATCGAGCACCTCGGCGAGGAGAAGGCCGTGCTCGTC
GGCCACGACGTGGGCGCGGTCGTGGCCTGGGCCGTGGCGCTCCGCCACCCGGAGCGCGTC
GAGAAGCTCGTCGCCATCAACATGTCTCACCCGGCGTGCTTCGACCGCGCGCTGCGGGAG
GACCCGTCGCAGCAGGCCGCGAGCCAGTACATGCACCTCTTCCGGAGCCGGTCCGCAGAG
GAGCACCTCTCCCGGAACCAGTACGGCTTTCTGCGGGAGATCTTCCTCGAGCCAGGGCTG
TCGCAGGGATATTTCAGCGAAGCGGACGTCCGTGTCTACCTGGAGGCGTTCGCGCAGCCG
GGCGCGATCACGGGCGGGCTCAACATCTACCGCGCGGCGCAGATCGGGCCTCCAGGCCCG
GGCCAGCCCGTCGGGGGGAGCAACCTCACCCGGGGTCTCCGCTCGCTCACCGTCTCCGTG
CCTGTCCTCGTGATCTGGGGCGAGCGCGATCCGTACCTCTTGCCCGCGAGCAACCTCGCC
GGGATCGAGCTTTACGTCCCCGATCTGAGGGTGATGCGCGTGCCCGAGGCGACCCACTGG
ATCGTCCACGAGGAGCCCGAGATCGTCAACGCGGCGATCCGCGGCTTCCTCGCGCGCTAG
MLPRNDTTHAHADVNGVRLHYASRGAGKLILFIHGFPELWYAWKRQLFDFGRHHRAVALD
QRGYNLSSKPSAVDAYGIDLLAADIAALIEHLGEEKAVLVGHDVGAVVAWAVALRHPERV
EKLVAINMSHPACFDPALREDPSQQAASQYMHLFRSRSAEEHLSRNEYGFLREIFLEPGL
SQGYFSEADVRVYLEAFAQPGAITGGLNTYRAAQIGPPGPGQPVGGSNLTRGLRSLTVSV
PVLVIWGERDPYLLPASNLAGTELYVPDLRVMRVPEATHWIVHEEPEIVNAAIRGFLAR
orf 7
ATGAACACGACGCTCAAGCTCCACGAGGAGTACCCGCCGCCCGGCGAGGAAGACAGCATC
CGCCAGATCACCGAGATCATGAGGCGCAATTACGAGCAAGCTTACCCCGCGGGCGCCTCG
CCCGCGCTCCGCGGGGTGCACCCGAAGTCGCACGGCTGCGTGAGGGCCCACTTCGTCGTC
GACGAGGGCTTGCCCCGCGAGCTCCGCCATGGCGTCTTCCGGGAGCCGCGCGTCTACCCG
GCGTGGGTCCGGTTCTCGTCGACCTCCTCGCGCGTCCAGTCGGACATGAAGCGCGACTCC
CGCTGCATGGCGATCAAGCTGCTCGGCGTCGAGGGGGAGAAGATCCTCGACGGCGAGAAG
GACGCGACGACGCAGGATTTCCTGATGGGGAACACGGACGTGTTCTTCTCGCGGAACATC
GCGGACTACGTCGAGCTCATGTCTGCCATGAGCGCGGGCAAGCCCCTCTCGTATTTCTGC
TCGCTCCGCCCGCCCCGCCTCCGGCTGCGCGAGCTCATGAACTACCTCTCCGTCGTGCTC
AAGCCGGTGAAGAACCCCCTGCACGCCCGGTATTTCAGCCAGACGCCGTTCCGGCTCGGC
GCGCGGGCGATGAAGTTCTGCGTGGTCCCCCGGCCCTGCGGCGCGCCCGGCGTCGGGGTC
GAGCCGGGCGACGACGCGCTCAAGCAGGCGGTGGCGCGGCAGCTCGAGGGGGGAGACTGG
ATGTTCGACTTCCTCGTGCAGCTCCAGGCCCACCCGACGAAGACGCCCATCGAGGATCCG
ACGATTCGCTGGAGCGAGGAACTGTCGCCGTTCACCAAGGTCGCGACGATGGTCATCCCG
GCGCAGCGCCTCGACCTCCCGGGACAGGCGGAGTTCGAGGAGAACCTCTCGTTCACGCCC
TGGCACGCCCTGCCGGCGCACCGGCCGCTCGGCGGGCTGAACCGCGCCCGGCGCGCGGTC
TACGAGGCGATATCGAAGCTCCGCCACGAGAGGAACGGGGCTCGCCGCGAGGAGCCGGCG
GCGCCGCGCGCCGGGCGCGCGCGCGCGCCGTCGAGCGCAGGGCCGGCGCGGCTCGGACTC
GGGTGA
MNTTLKLHEEYPPPGEEDSIRQITETMRRNYEQAYPAGASPALRGVHPKSHGCVRAHFVV
DEGLPRELRHGVFREPRVYPAWVRFSSTSSRVQSDMKRDSRCMATKLLGVEGEKILDGEK
DATTQDFLMGNTDVFFSRNIADYVELMSAMSAGKPLSYFCSLRPPRLRLRELMNYLSVVL
KPVKNPLHARYFSQTPFRLGARAMKFCVVPRPCGAPGVGVEPGDDALKQAVARQLEGGDW
MFDFLVQLQAHPTKTPIEDPTIRWSEELSPFTKVATMVIPAQRLDLPGQAEFEENLSFTP
WHALPAHRPLGGLNRARPAVYEATSKLRHERNGARREEPAAPPAGPAPAPSSAGPARLGL
G
orf 8-partial sequence
CGGCCGCAATTAACCCTCACTAAAGGGATCATGCTCACTGCGAGCCTGTTCGTGAGCGCG
GTCGTGCAGGTGATCGTCAACGTCGGACGCTACCGTTCCTGCGTCCTCGTCCACATGCTG
CGGTCGCTCATGCACGGCTTCCGCGCGCTCCACAAGGACCCCGACGTCGTCCATCCGGCG
ACCGGGCGCCCGGCGCATGGCGCGCTCGCCAGGAACGAAGCCTGTGAAATGCGCTTCGTG
CACGAGGTGCTCTCCGATCCGGTCCTGCACGCGCGCAGCGCGAAGCTCGCGTACGGCTCG
GACCCCGAGCGGCTCGCGGGCCTCGTCGAGTACATCGACGTCGAGGACCTGATCAGCATC
GCCACCGGCGCCTCGGCGCCGGCCGACGGGGACGGGCGCGAGCTCCTCTTGCCGAGCCGC
TGGGTGAGCTGCGGCCACGGCGGCTCCAGCGCGCCGAAGCGCTACGCGACGCTGGAGCAG
TTCTCCACGTACGTGCGCCGCTGGTTTCCCACCCTCGAGGGCACGGCGTCGCAGACGTTC
AAGCTGCGCGTCCGGCGGCTGACGCTCCTCGTCTCGATGATGGTGGTCATCCTCTTCAAC
TTCGACGGCTTCCAGGTGCTCGCGCGGCTCCACCAGAGCGGCGCCGCGCGCGCGCAGGTG
GCCGCCCAGGCGGACACGGTGGCGACCACCGCGGCGCGCCTGGGCGAGCTCCCGGCCGGC
GCGCCGGCCGAGCTCCCCGACGCCACGCTCGAGGAGCTCGGGCTCGAGATTCAGAAGACG
GCGACCCTCCTCGACGAGGCGAACCTCGGCGTCGGCTGGCAGCAGAGCTGGATCGTCCAG
CGGTTCCGCGCGTACCGCCACGATATCCTGGCGCCCCCGCCGACCTCGCTGGAGCTCCTC
CAGGACACGCTGTTCTGGCTGGCCGGGCTCGCCTTCTCGTGCGGGTTGCTCTCCCTGGGA
GCGCCCTTCTGGGTCACCACGTTCGCTCGGCTCATCCAGATGCGCAACGAGGTGCAGCAC
CGGAAGCGCCAGGAGAGCGCGTCCGGCGTGAAGGTCGCGAGCACCGCCCTGCCGTTCCCC
GCGCGCGAGCGCGCCGTCGCCAAGCCTTCCTGA
RPQLTLTKGIMLTASLFVSAVVQVIVNVGRYRSCVLVHMLRSLMHGFPALHKDPDVVDPA
TGRPAHGALARNEACEMRFVHEVLSDPVLHARSAKLAYGSDPERLAGLVEYIDVEDLTSI
ATGASAPADGDGRELLLPSRWVSCGHGGSSAPKRYATLEQFSTYVRRWPPTLEGTASQTF
KLRVRRLTLLVSMMVVILFNFDGFQVLARLHQSGAARAQVAAQADTVATTAARLGELPAG
APAELPDATLEELGLEIQKTATLLDEANLGVGWQQSWIVQRFRAYRHDILAPPPTSLELL
QDTLFWLAGLAFSCGLLSLGAPFWVTTFARLIQMRNEVQHRKRQESASGVKVASTALPFP
AREPAVAKPS

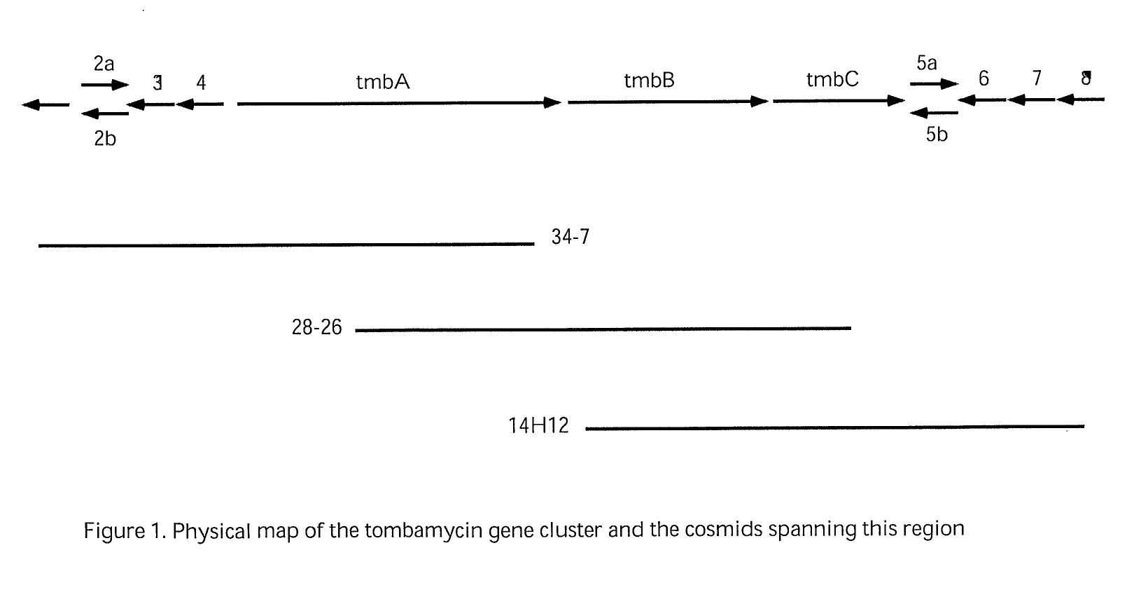

The genes of the tmbA gene cluster can be isolated from the cosmids of the invention shown in FIG. 1 or from Sorangium cellulosum genomic DNA.

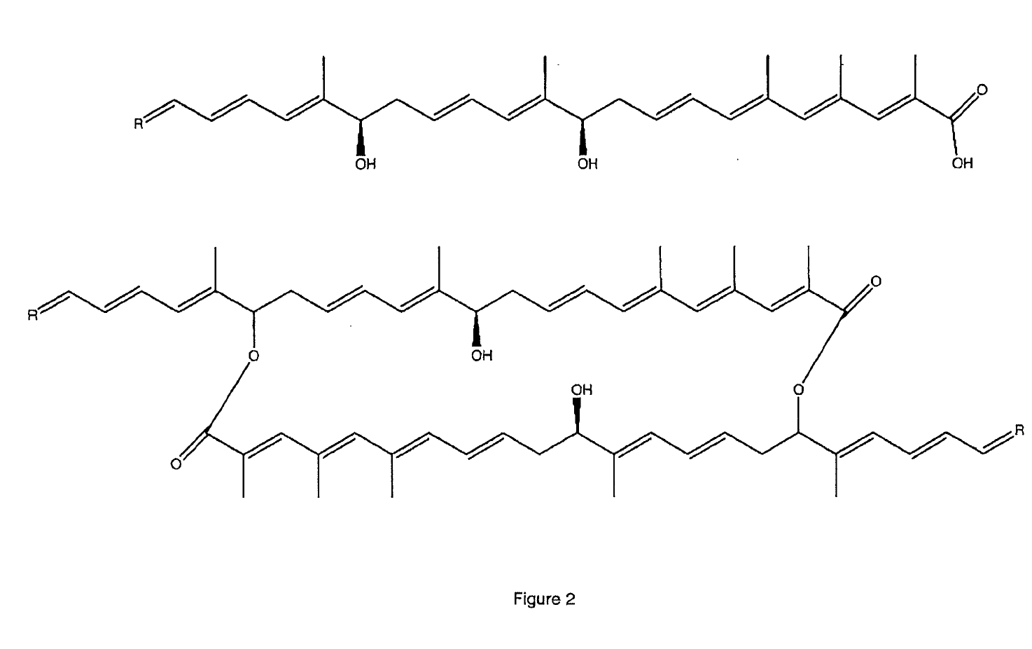

The gene products of the tmbA cluster can be used to synthesize the polyketide tombamycin, the structure of which is shown in FIG. 2. Tombamycin can be dimerized to produce the dimeric form of tombamycin, the structure of which is also shown in FIG. 2.

The invention having now been described by way of written description those of skill in the art will recognize that the invention can be practiced in a variety of embodiments and that the foregoing description and examples are for purposes of illustration and not limitation of the following claims.

1. A recombinant DNA vector that comprises all or a portion of one or more genes in the tmbA gene cluster.
